(19) United States
(12) Patent Application Publication
(10) Pub. No.: US 2009/0137191 A1
(43) Pub. Date: May 28, 2009

(54) COPPER CMP POLISHING PAD CLEANING COMPOSITION COMPRISING OF AMIDOXIME COMPOUNDS

(76) Inventor: Wai Mun Lee, Fremont, CA (US)

Correspondence Address: MORGAN LEWIS & BOCKIUS LLP, 1111 PENNSYLVANIA AVENUE NW, WASHINGTON, DC, 20004, US

(21) Appl. No.: 12/260,602

(22) Filed: Oct. 29, 2008

Related U.S. Application Data
(60) Provisional application No. 61/000,727, filed on Oct. 29, 2007.
(60) Provisional application No. 61/006,227, filed on Dec. 31, 2007.

(51) Int. Cl. B24B 53/02; B24B 1/00; C07C 249/04; B24B 7/20

(52) U.S. Cl. 451/36; 451/56; 451/41; 564/268

(57) ABSTRACT

The present invention relates to methods of using amidoxime compositions for cleaning polishing pads, particularly after chemical mechanical planarization or polishing is provided. A polishing pad is cleaned of Cu CMP by-products, subsequent to or during planarizing a wafer, to reduce pad-glazing by applying to the polishing pad surface a composition comprising an aqueous amidoxime compound solution in water.

### CROSS-REFERENCE TO RELATED APPLICATIONS

This application claims the benefit of U.S. Provisional Application No. 61/000,727, filed Oct. 29, 2007, and U.S. Provisional Application No. 61/006,227, filed Dec. 31, 2007, both of which are incorporated herein by reference in their entirety.

### FIELD OF THE INVENTION

The present invention discloses a method and a system for cleaning a chemical-mechanical polishing (CMP) pad. More specifically, the present invention discloses a method of cleaning a polishing pad surface subsequent to chemical-mechanical polishing a wafer surface. The method including applying to the polishing pad surface a cleaning composition comprising one or more compounds having at least one amidoxime functional group in water. The composition is then allowed to react with a residue that may be on the pad to produce water soluble by-products. Next, the pad surface is rinsed with water, preferably deionized water, to substantially remove the by-products. A mechanical conditioning operation is subsequently performed on the surface of the pad. In one example, the wafer surface can be a metal, such as copper or a copper alloy.

### BACKGROUND OF THE INVENTION

The present invention relates generally to semiconductor processing, particularly chemical-mechanical polishing (CMP). The present invention is applicable to polishing pads employed in CMP, particularly conditioning the polishing pad to reduce defects.

Current semiconductor processing typically comprises forming an integrated circuit containing a plurality of conductive patterns on vertically stacked levels connected by vias and insulated by inter-layer dielectrics. As device geometry plunges into the deep sub-micron range, chips comprising five or more levels of metallization are formed.

In manufacturing multi-level semiconductor devices, it is necessary to form each level with a high degree surface planarity, avoiding surface topography, such as bumps or areas of unequal elevation, i.e., surface irregularities. In printing photolithographic patterns having reduced geometry dictated by the increasing demands for miniaturization, a shallow depth of focus is required. The presence of surface irregularities can exceed the depth of focus limitations of conventional photolithographic equipment. Accordingly, it is essential to provide flat planar surfaces in forming levels of a semiconductor device. In order to maintain acceptable yield and device performance, conventional semiconductor methodology involves some type of planarization or leveling technique at suitable points in the manufacturing process.

A conventional planarization technique for eliminating or substantially reducing surface irregularities is CMP wherein abrasive and chemical action is applied to the surface of the wafer undergoing planarization. The polishing pad is employed together with a chemical agent to remove material from the wafer surface.

FIG. 1 is a schematic top plan view of a conventional CMP apparatus 11 comprising a rotatable platen 15 on which is mounted a polishing pad 17 for polishing semiconductor substrate S. The polishing pad 17 can be a conventional slurry-type pad having a plurality of concentric circumferential grooves 19 as illustrated, or a fixed abrasive-type polishing pad.

CMP apparatus 11 further comprises a pivot arm 21, a holder or conditioning head 23 mounted to one end of the pivot arm 21, a pad conditioner 25, such as a pad embedded with diamond crystals, mounted to the underside of the conditioning head 23, a slurry source such as a slurry/rinse arm 27, and a substrate mounting head 29 operatively coupled to platen 15 to urge substrate S against the working surface of polishing pad 17. Pivot arm 21 is operatively coupled to platen 15, and maintains conditioning head 23 against the polishing pad 17 as the pivot arm 21 sweeps back and forth across the radius of polishing pad 17 in an arcing motion. Slurry/rinse arm 27 is stationarily positioned outside the sweep of the pivot arm 21 and the conditioning head 23 coupled thereto.

In operation, the substrate S is placed face down beneath the substrate mounting head 29, and the substrate mounting head 29 presses the substrate S firmly against the polishing pad 17. Slurry is introduced to the polishing pad 17 via slurry/rinse arm 27, and platen 15 rotates as indicated by arrow R₁. Pivot arm 21 scans from side to side in an arcing motion as indicated by arrow S₁.

When the pad is grooved, then grooves 19 channel the slurry (not shown) between the substrate S and the polishing pad 17. The semi-porous surface of the polishing pad 17 becomes saturated with slurry which, with the downward force of the substrate mounting head 29 and the rotation of the platen 15, abrades and planarizes the surface of the substrate S. The diamond crystals (not shown) embedded in the rotating conditioner 25 continually roughens the surface of the polishing pad 17 to ensure consistent polishing rates. Pad cleaning must be performed frequently to clean polishing residue and compacted slurry from the polishing pad 17.

Conventional pad cleaning techniques employ rinsing wherein the substrate mounting head 29 is removed from contact with the polishing pad 17, the supply of slurry from the slurry/rinse arm 27 is turned off, and a rinsing fluid such as deionized water is supplied via the slurry/rinse arm 27. However, merely rinsing the polishing pad following CMP is often ineffective in removing polishing residues, particularly after CMP of metal films, because polishing by-products stick to the polishing pad.

Conventional polishing pads employed in abrasive slurry processing typically comprise a grooved porous polymeric surface, such as polyurethane, and the abrasive slurry varied in accordance with the particular material undergoing CMP. Basically, the abrasive slurry is impregnated into the pores of the polymeric surface while the grooves convey the abrasive slurry to the wafer undergoing CMP. Another type of polishing pad is a fixed abrasive pad wherein abrasive elements are mounted on a backing. When conducting CMP with a fixed abrasive pad, a chemical agent without abrasive particles is applied to the pad surface.

When conducting CMP on a metal-containing surface, e.g., Cu or a Cu alloy, the working or polishing surface of the polishing pad undergoes changes believed to be caused by, inter alia, polishing by-products resulting from the reaction of metal being removed from the wafer surface, such as Cu, with components of the CMP slurry or chemical agent, e.g., oxidizer, complexing agents and inhibitors. Such by-products typically deposit onto the polishing pad and accumulate causing a colored stain or glazed area. Such a surface exhibits a lower coefficient of friction and, hence, a substantially lower material removal rate by adversely impacting polishing uniformity and increasing polishing time. In addition, such glazing causes scratching of the wafer surface. Conventional approaches to remedy pad glazing include pad conditioning, as with nylon brushes or diamond disks for removing the deposited by-products from the polishing pad surface. However, such a conventional remedial approach to the glazing problem is not particularly effective in completely removing glazing. Pad conditioning with a diamond disk also greatly reduces pad lifetime.

There exists a need for methodology enabling the planarization of a wafer surface containing Cu or Cu alloy with reduced pad glazing. There exists a particular need for a methodology enabling CMP of a wafer surface containing Cu or Cu alloys at high production throughput.

Further, most formulations used in the CMP process contain complexing agents, sometimes called chelating agents. Much metal-chelating functionality is known which causes a central metal ion to be attached by coordination links to two or more nonmetal atoms (ligands) in the same molecule. Heterocyclic rings are formed with the central (metal) atom as part of each ring. When the complex becomes more soluble in the solution, it functions in the cleaning process. If the complexed product is not soluble in the solution, it becomes a passivating agent by forming an insoluble film on top of the metal surface. The current complexing agents in use, such as, glycolic acid, glyoxylic acid, lactic acid, phosphonic acid, are acidic in nature and have a tendency to attack the residue and remove both metals and metal oxides, such as copper and copper oxide. This presents a problem for formulators where a chelating function is sought but only selectively to metal oxide and not the metal itself, e.g., in an application involving metal, such as copper. Accordingly, there is a need for complexing agents that are not aggressive toward metal substrates, while effectively providing for the chelation of metal ions residue created during the manufacturing processes.

In some cases, the biodegradability is also unsatisfactory. Thus, EDTA proves to have inadequate biodegradability in conventional tests, as does PDTA or HPDTA and corresponding aminomethylenephosphonates which, moreover, are often undesirable because of their phosphorus content. Phosphorus is also a dopant in semiconductor devices; therefore, it is desirable to have CMP pad cleaning solutions with non-phosphor containing compounds.

The present invention addresses the aforementioned problems.

### SUMMARY OF THE INVENTION

An aspect of the present invention is a method of cleaning a polishing pad surface to prevent or substantially reduce pad glazing stemming from conducting CMP on a wafer surface containing Cu or Cu alloy.

One embodiment of the invention is a method of cleaning a polishing pad surface subsequent to chemical-mechanical polishing (CMP) a wafer surface containing copper (Cu) or a Cu-based alloy comprising applying to the polishing pad surface a cleaning composition comprising from about 2 ppm to about 50 percent by weight of one or more compounds having at least one amidoxime functional group in water (e.g. deionized water), optionally, with an acid or a base in amount such that the composition effectively solubilizes the copper and copper alloy. In one embodiment, the composition is applied to a rotating polishing pad at a flow rate of about 100 to about 600 ml/min. In that embodiment, the composition may be applied to the polishing pad for about 3 seconds to about 20 seconds after conducting CMP on each of a plurality to wafers having a surface comprising Cu or Cu alloy.

Another embodiment of the invention is a method comprising: (a) conducting chemical-mechanical polishing (CMP) on a first wafer surface of a first wafer containing copper (Cu) or a Cu-based alloy on a surface of a polishing pad; (b) removing the first wafer from the pad; (c) applying to the polishing pad surface a cleaning composition, wherein the cleaning composition is a solution comprising about 2 ppm to about 50 percent by weight of one or more compounds having at least one amidoxime functional group in water (e.g. deionized water), optionally, with an acid or a base in amount such that the composition effectively solubilize the copper and copper alloy; (d) rinsing the polishing pad surface with water to remove any cleaning composition on the polishing surface; (e) conducting CMP on a second wafer; and then (f) repeating steps (b) through (e) one or more times. In one embodiment, the water is deionized water. In another embodiment, the cleaning is applied to a rotating polishing pad at a flow rate of about 100 to about 600 ml/min. In that embodiment, the cleaning composition is applied to the rotating polishing pad for about 3 seconds to about 20 seconds. In one embodiment, the one or more compounds containing at least one amidoxime functional group may be present in the polishing composition in an amount of about 0.001 percent by weight to about 5 percent by weight. In another embodiment, the one or more compounds containing at least one amidoxime functional group may be present in the polishing composition in an amount of about 2 ppm to about 50 percent by weight. In yet another embodiment, the cleaning composition may further contain one or more oxidizers and one or more surface-active agents; preferably the surface-active agents include at least one member selected from the group consisting of anionic surfactants, Zwitter-ionic surfactants, multi-ionic surfactants, and combinations thereof. In one embodiment of the invention, the surfactant may be selected from sodium salts of polyacrylic acid, potassium oleate, sulfosuccinates, sulfosuccinate derivatives, sulfonated amines, sulfonated amides, sulfates of alcohols, alkylanyl sulfonates, carboxylated alcohols, alkylamino propionic acids, alkyliminodipropionic acids, and combinations thereof. In another embodiment, the surfactant is present in an amount between about 0.001 and about 10 percent by weight of the composition. In yet another embodiment, the cleaning composition further comprises a compound with oxidization or reduction potential. Optionally, the composition may be further diluted with water (e.g. about 10 to 500 times) prior to applying it to the polishing pad surface.

Yet another embodiment of the invention is a method of cleaning a surface of a polishing pad, comprising:

(a) conducting chemical-mechanical polishing (CMP) on a first wafer on the surface of the polishing pad;

(b) removing the first wafer from the polishing pad;

(c) applying to the polishing pad surface a cleaning composition, wherein the cleaning composition is a solution comprising from about 2 ppm to about 50 percent by weight of one or more compounds having at least one amidoxime functional group in deionized water, optionally, with an acid or a base in amount such that the composition effectively solubilize the copper and copper alloy; and

(d) cleaning the polishing pad surface with the cleaning composition. The cleaning composition may further contain hydrogen peroxide or hydroxylamine, with the mixing ratio of the one or more compounds having at least one amidoxime functional group: hydrogen peroxide or hydroxylamine: water ranging from about 1:4:20 to about 1:1:5, the waiting time for allowing the solution to react with the residue being between about 30 to about 180 seconds, and the solution being applied to the polishing pad at a heated temperature between about 40° C. and about 80° C. In one embodiment, the surface of the first wafer to be polished is substantially comprised of an oxide, and the cleaning composition, optionally, further contains H₂O₂ or hydroxylamine.

In one embodiment of the invention, the one or more compounds having at least one amidoxime functional group have at least one of the following structures:

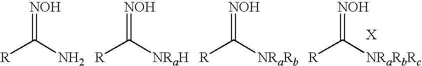

or tautomers thereof, wherein R, Rₐ, R_{b} and R_{c} are independently alkyl, heteroalkyl, aryl or heteroaryl.

In another embodiment of the invention, the one or more compounds having at least one amidoxime functional group have the following structure:

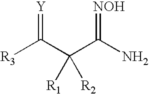

wherein R₁, R₂ and R₃ are independently hydrogen, heteroatoms, heterogroups, alkyl, heteroalkyl, aryl or heteroaryl; and wherein Y is O, NH or NOH.

In another embodiment of the invention, the one or more compounds having at least one amidoxime functional group have the following structure:

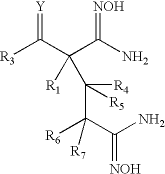

wherein R₁, R₂ and R₃ are independently hydrogen, heteroatoms, heterogroups, alkyl, heteroalkyl, aryl or heteroaryl,

wherein Y is O, NH or NOH, and

wherein R₄, R₅, R₆ and R₇ are independently hydrogen, heteroatoms, heterogroups, alkyl, heteroalkyl, aryl or heteroaryl.

In another embodiment, the one or more compounds having at least one amidoxime functional group are selected from the group consisting of 1,2,3,4,5,6-hexakis-O-[3-(hydroxyamino)-3-iminopropyl Hexitol; 3,3′,3″,3′″-(ethane-1,2-diylbis(azanetriyl))tetrakis(N″-hydroxypropanimidamide); 3,3′-(ethane-1,2-diylbis(oxy))bis(N′-hydroxypropanimidamide); 3-(diethylamino)-N′-hydroxypropanimidamide; 3,3′-(piperazine-1,4-diyl)bis(N′-hydroxypropanimidamide); 3-(2-ethoxyethoxy)-N′-hydroxypropanimidamide; 3-(2-(2-(dimethylamino)ethoxy)ethoxy)-N′-hydroxypropanimidamide; N′-hydroxy-3-(phenylamino)propanimidamide; 3,3′,3″-nitrilotris(N′-hydroxypropanimidamide); 3,3′-(2,2-bis((3-(hydroxyamino)-3-iminopropoxy)methyl)propane-1,3-diyl)bis(oxy)bis(N-hydroxypropanimidamide); 3,3′-(2,2′-(methylazanediyl)bis(ethane-2,1-diyl)bis(oxy))bis(N′-hydroxypropanimidamide); N,N-bis(3-amino-3-(hydroxyimino)propyl)acetamide; 3,3′-(2-(N′-hydroxycarbamimidoyl)phenylazanediyl)bis(N′-hydroxypropanimidamide); 3,3′-(2,2′-(3-amino-3-(hydroxyimino)propylazanediyl)bis(ethane-2,1-diyl))bis(oxy)bis(N′-hydroxypropanimidamide) and mixtures thereof.

In yet another embodiment, the one or more compounds having at least one amidoxime functional group are selected from the group consisting of 3,3′,3″,3′″-(ethane-1,2-diylbis(azanetriyl))tetrakis(N′-hydroxypropanimidamide); 3,3′-(ethane-1,2-diylbis(oxy))bis(N′-hydroxypropanimidamide); 1,2,3,4,5,6-hexakis-O-[3-(hydroxyamino)-3-iminopropyl Hexitol; 3,3′-(2,2-bis((3-(hydroxyamino)-3-iminopropoxy)methyl)propane-1,3-diyl)bis(oxy)bis(N-hydroxypropanimidamide); N′,2-dihydroxyacetimidamide and mixtures thereof.

In yet another embodiment, the one or more compounds having at least one amidoxime functional group are derived from the reaction of a nitrile with hydroxylamine.

According to the present invention, the foregoing and other aspects are achieved in part by a method of cleaning a polishing pad surface subsequent to CMP a wafer surface containing Cu or a Cu alloy, the method comprising applying to the polishing pad surface a cleaning composition comprising: about 2 ppm to about 50 percent by weight of one or more compounds with at least one amidoxime functional group in water, preferably deionized water, optionally with an acid or a base in an amount such that the composition effectively solubilize the copper and copper alloy. The cleaning composition can further include a compound with an oxidation or reduction potential.

Another aspect of the present invention is a method comprising the sequential steps: (a) conducting CMP on a first wafer surface containing Cu or a Cu alloy on a surface of a polishing pad; (b) applying to the polishing pad surface a cleaning composition comprising: about 2 ppm to about 50 percent by weight of one or more compounds with at least one amidoxime functional group in deionized water, optionally with an acid or a base in amount such that the composition effectively solubilize the copper and copper alloy; (c) rinsing the polishing pad surface with water to remove any cleaning composition on the polishing pad surface; (d) conducting CMP on a second wafer surface; and (e) repeating steps (b) through (d).

Another aspect of the present invention is a method comprising: (a) conducting chemical-mechanical polishing (CMP) on a first wafer surface of a first wafer containing copper (Cu) or a Cu-based alloy on a surface of a polishing pad; (b) removing the first wafer from the pad; (c) applying to the polishing pad surface a cleaning composition, wherein the cleaning composition is a solution comprising about 2 ppm to about 50 percent by weight of one or more compounds having at least one amidoxime functional group in water, optionally, with an acid or a base in amount such that the composition effectively solubilize the copper and copper alloy; (d) rinsing the polishing pad surface with water to remove any cleaning composition on the polishing surface; (e) conducting CMP on a second wafer; and then (9 repeating steps (b) through (e) one or more times.

Another aspect of the present invention is an apparatus for conducting a CMP on a wafer surface containing Cu or Cu alloy with significantly reduced pad glazing.

A further aspect of the present invention is an apparatus for conducting CMP on a wafer surface containing Cu or a Cu alloy, the apparatus comprising: a platen; a polishing sheet or pad mounted on the platen; a first dispenser adapted to dispense a cleaning composition on a working surface of the polishing sheet or pad; and a source of the cleaning composition coupled to the first dispenser, the cleaning composition comprising: about 2 ppm to about 50 percent by weight of one or more compounds with at least one amidoxime functional group in deionized water, optionally with an acid or a base in amount such that the composition effectively solubilize the copper and copper alloy.

Another aspect of the present invention is a method of cleaning a surface of a polishing pad, the method including the steps of (a) conducting chemical-mechanical polishing (CMP) on a first wafer on the surface of the polishing pad; (b) removing the first wafer from the polishing pad; (c) applying to the polishing pad surface a cleaning composition, wherein the cleaning composition is a solution comprising from about 2 ppm to about 50 percent by weight of one or more compounds having at least one amidoxime functional group in deionized water, optionally, with an acid or a base in amount such that the composition effectively solubilize the copper and copper alloy; and (d) cleaning the polishing pad surface with the cleaning composition.

Embodiments of the present invention comprise conducting CMP on a plurality of wafers having a surface containing Cu or a Cu alloy. After each wafer is subjected to CMP, the polishing pad surface is cleaned with a cleaning solution containing one or more compounds with at least one amidoxime functional group in deionized water, optionally including an acid such as phosphoric acid, acetic acid and sulfuric acid, or a base, such as potassium, sodium or ammonium hydroxide. The cleaning solution is then rinsed away from the polishing pad surface with pressurized water. Pad conditioning can also be implemented before, during and/or after applying the cleaning solution. Embodiments of the present invention further include an apparatus containing a first dispenser for dispensing the cleaning solution and, a second dispenser for rinsing the polishing pad surface after application of the cleaning solution, and a computer programmed to implement CMP, polishing pad surface cleaning and polishing pad surface rinsing.

In certain embodiments, the composition can be further diluted with water prior to applying it to the polishing pad surface. The dilution factor can be from about 10 to 500.

Additional aspects of the present invention will become readily apparent to those skilled in this art from the following detailed description, wherein embodiments of the present invention are described, simply by way of illustration of the best mode contemplated for carrying out the present invention. As will be realized, the present invention is capable of other and different embodiments, and its several details are capable of modifications in various obvious respects, all without departing from the present invention. Accordingly, the drawings and description are to be regarded as illustrative in nature, and not as restrictive. The aspects of the present invention may be realized and obtained as particularly pointed out in the appended claims.

### BRIEF DESCRIPTION OF DRAWINGS

FIG. 1 schematically illustrates a conventional CMP apparatus.

FIG. 2 schematically illustrates a CMP apparatus in accordance with an embodiment of the present invention.

FIG. 3 shows a plot of copper thickness lost vs. time for cleaning compositions comprising hydrogen peroxide, an amidoxime compound and a mixture of hydrogen peroxide and an amidoxime compound.

FIG. 4 shows the result of an ESCA analysis of the copper surface without any treatment, indicating a high concentration of Cu(II) oxide.

FIG. 5 shows the efficacy Cu(II) oxide removal by the amidoxime solution.

FIG. 6 shows that amidoxime compounds also inhibit the growth of Cu(II) oxide.

FIG. 7 is an Auger depth profile analysis of the cleaning treated copper surface. The result suggests that the Cu(I) and Cu(II) oxide thickness have not increased.

FIG. 8 shows a Copper Pourbaix diagram.

### DETAILED DESCRIPTION OF THE INVENTION

The present invention addresses and solves the pad glazing problem attendant upon conducting chemical-mechanical polishing (CMP) on a wafer surface containing Cu and/or Cu alloys. As employed throughout this disclosure, the symbol Cu is intended to encompass high purity elemental copper as well as copper-based alloys, e.g., copper alloys containing about 80% of copper and greater. As also employed throughout this disclosure, the expression “ex situ” treatment is intended to encompass polishing pad treatment conducted while a wafer is not in contact with the polishing pad and/or undergoing CMP.

Pad glazing attendant upon conducting CMP of a wafer surface containing Cu adversely impacts the uniformity and polishing rate of CMP. Accordingly, pad conditioning is conventionally conducted, notably with a diamond disk. It is believed that pad glazing stems from the accumulation of polishing by-products, particularly Cu-complexes with slurry components, such as complexing agents and inhibitors.

The present invention addresses and solves the pad glazing problem attendant upon conducting CMP of a wafer surface containing Cu by addressing the source of the problem, i.e., by removing the Cu-containing polishing by-products before such polishing by-products transform into a glazing on the pad surface. In accordance with embodiments of the present invention, after conducting CMP on a wafer surface containing Cu, the polishing pad working surface is treated with a cleaning composition comprising about 2 ppm to about 50 percent by weight of one or more compounds with at least one amidoxime functional group in deionized water, optionally with an acid or a base in amount such that the composition effectively solubilize the copper and copper alloy. Subsequent to cleaning, the polishing pad surface is rinsed with water, as by water under pressure, to remove the cleaning solution prior to initiating CMP on a subsequent wafer.

Embodiments of the present invention further include optionally conditioning the pad surface to remove any glazing which may occur, as by employing a conventional disk, before, during and/or after treatment with the cleaning composition

Embodiments of the present invention comprise treating the polishing pad surface with a cleaning solution containing one or more compounds with at least one amidoxime functional group in deionized water, optionally including an acid such as phosphoric acid, acetic acid and sulfuric acid, or a base, such as potassium, sodium or ammonium hydroxide. Given the present disclosure and objectives, the optimum flow rate and time for treating a polishing pad surface can be determined in a particular situation. For example, it was found suitably to apply the cleaning solution to a rotating polishing pad at a flow rate of about 100 to about 600 ml/min, e.g., about 100 to about 200 ml/min, for about 3 to about 20 seconds. The solution can then be removed from the polishing pad surface by applying pressurized deionized water for about 2 to about 20 seconds.

It was found that the sequential treatment of a polishing pad surface with a cleaning solution containing one or more compounds with at least one amidoxime functional group, an acid or a base and water followed by rinsing with water significantly reduces pad glazing, increases wafer to wafer rate uniformity and reduces wafer scratches. The exact mechanism underpinning the significant reduction in pad glazing attendant upon employing a cleaning solution in accordance with embodiments of the present invention is not known with certainty. However, it is believed that amidoxime compounds form water soluble complexes with Cu and/or the Cu-containing CMP by-products and such complexes dissolve in water. Upon subsequent rinsing with water, the remaining cleaning composition and solubilized by-products are removed, thereby preventing and/or significantly reducing the formation of pad glazing in an efficient, cost effective manner.

Embodiments of the present invention, therefore, comprise a method of conducting CMP on a plurality of individual wafers having a surface containing Cu. After each wafer is planarized, the polishing pad surface is treated with a cleaning solution and then rinsed, in accordance with embodiments of the present invention, to prevent and/or significantly reduce pad glazing, thereby improving wafer to wafer rate uniformity and reducing wafer scratches

Embodiments of the present invention further include polishing apparatus comprising various types of platens, including linear platens and apparatuses comprising at least one platen, a polishing pad or sheet mounted on the platen, a first dispenser for dispensing a cleaning solution containing one or more compounds with at least one amidoxime functional group in deionized water, optionally with an acid or a base in amount such that the composition effectively solubilize the copper and copper alloy, a second dispenser for dispensing water, e.g., pressurized water, on the polishing pad surface to remove the cleaning solution and dissolved CMP by-products prior to initiating CMP of a subsequent wafer. An apparatus in accordance with embodiments of the present invention can also include a controller programmed for dispensing the cleaning solution onto the polishing pad surface and for rinsing the polishing pad surface to remove the remaining cleaning solution and dissolved polishing by-products prior to initiating CMP of a subsequent wafer. The apparatus can also be programmed for implementing polishing pad conditioning before, during and/or after treatment of the pad surface with a cleaning solution.

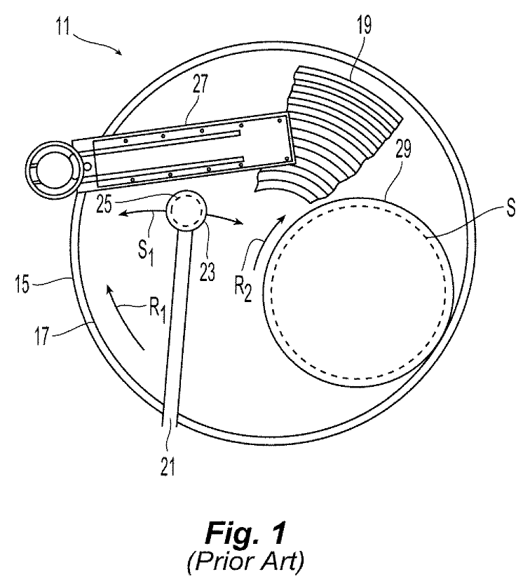

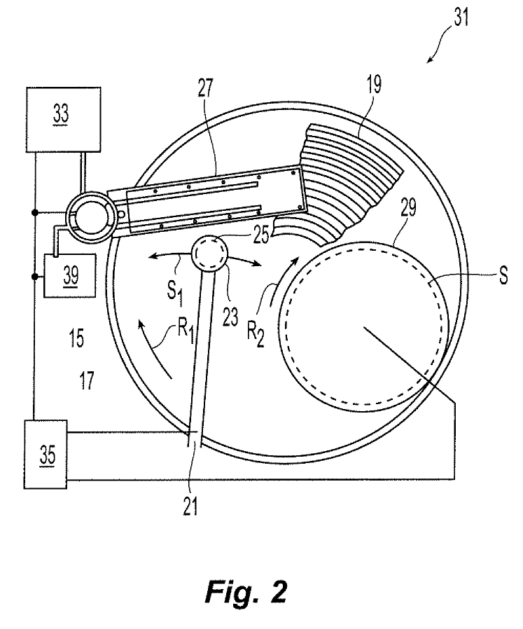

An apparatus in accordance with an embodiment of the present invention is schematically illustrated in FIG. 2. The inventive apparatus 31 comprises many components described with reference to the conventional apparatus 11 illustrated in FIG. 1. However, the inventive apparatus 31 further comprises a source of cleaning solution 33, a cleaning solution containing one or more compounds with at least one amidoxime functional group in deionized water, optionally including an acid such as phosphoric acid, acetic acid and sulfuric acid, or a base, such as potassium, sodium or ammonium hydroxide, coupled to slurry/rinse arm 27, and a controller 35 coupled to the platen 15, pivot arm 21, slurry/rinse arm 27 and the source of cleaning solution 33. Additionally, source of rinsing fluid 39 (e.g., a source of deionized water) is coupled to the slurry/rinse arm 27 and the controller 35. Controller 35 can be programmed for controlling all aspects of operation, including CMP of a substrate S on polishing pad 17, conditioning the polishing pad 17 via pivot arm 21, dispensing (via slurry/rinse arm 27) cleaning solution from the source of cleaning solution 33, and dispensing rinsing fluid from the source of rinsing fluid 39.

In operation, a substrate S is placed face down beneath the substrate mounting head 29, and the substrate mounting head 29 presses the substrate S firmly against the polishing pad 17. Slurry is introduced to the polishing pad 17 via slurry/rinse arm 27, and platen 15 rotates as indicated by arrow R₁. Pivot arm 21 scans from side to side in an arcing motion as indicated by arrow S₁.

If the pad is grooved, the grooves 19 channel the slurry (not shown) between the substrate S and the polishing pad 17. The semi-porous surface of the polishing pad 17 becomes saturated with slurry which, with the downward force of the substrate mounting head 29 and the rotation of the platen 15, abrades and planarizes the surface of the substrate S. The diamond crystals (not shown) embedded in the rotating conditioner 25 continually roughen the surface of the polishing pad 17 to ensure consistent polishing rates, if necessary.

Unlike conventional pad cleaning techniques which merely use a rinsing fluid such as de-ionized water to remove slurry particles and polishing residue, the inventive apparatus 31 employs a cleaning solution having a chemistry adapted to improve pad cleaning. Specifically, the cleaning solution has a chemistry adapted to solubilize Cu-containing CMP residue on the surface of polishing pad 17 before glazing occurs. In this manner, even difficult to remove Cu-containing compounds in the solid state, can be cleaned from the polishing pad 17 in an efficient, cost effective manner. Subsequently, the surface of polishing pad 17 is rinsed as with pressurized deionized water dispensed from slurry/rinse arm 27.

The present invention advantageously significantly reduces polishing pad glazing at its source by solubilizing and removing Cu-containing CMP residue before glazing occurs on the polishing pad surface. The present invention can be implemented in a cost effective, efficient manner employing conventional materials and chemicals, with minor modifications to existing CMP devices. The present invention significantly improves wafer-to-wafer CMP rate uniformity and, at the same time, significantly reduces wafer scratches, in a cost effective and efficient manner.

The present invention is applicable to the manufacture of various types of semiconductor devices. The present invention is particularly applicable to manufacturing multi-level semiconductor devices having sub-micron features.

In the previous description, numerous specific details are set forth, such as specific materials, structures, chemicals, processes, etc., to provide a better understanding of the present invention. However, the present invention can be practiced without resorting to the details specifically set forth. In other instances, well known methodology, materials and features have not been described in detail in order not to unnecessarily obscure the present invention.

Only the preferred embodiment of the present invention and but a few examples of its versatility are shown and described in the present disclosure. It is to be understood that the present invention is capable of use in various other combinations and environments and capable of changes or modifications within the scope of the inventive concept as expressed herein.

Amidoxime Containing Compounds

The content of the amidoxime in the pad cleaner of the present invention is set preferably not less than 2 ppm and not greater than 50 percent by weight in deionized water. More preferably, it is set between 0.01 percent by weight and 20 percent by weight, more preferably between 1 percent by weight and 10 percent by weight.

A preferred source of the amidoxime group is from a nitrile compound that is derived from the cyanoethylation of a compound selected from the group consisting of sugar alcohols, hydroxy acids, sugar acids, monomeric polyols, polyhydric alcohols, glycol ethers, polymeric polyols, polyethylene glycols, polypropylene glycols, amines, amides, imides, amino alcohols, and synthetic polymers.

The reaction of nitrile-containing compounds with hydroxylamine is as follows, for example:

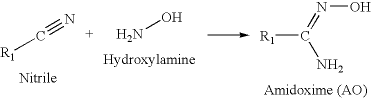

The amidoxime structure can be represented in their resonance form as illustrated below

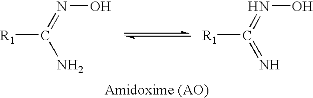

Amidoximes are made by the reaction of hydroxylamine with nitrile compounds. The most preferred compounds which undergo cyanoethylation include the following:
Compounds containing one or more —OH or —SH groups, such as water, alcohols, phenols, oximes, hydrogen sulphide and thiols.
Compounds containing one or more —NH— groups, e.g., ammonia, primary and secondary amines, hydrazines, and amides.
Ketones or aldehydes possessing a —CH—, —CH₂—, or CH₃ group adjacent to the carbonyl group.
Compounds such as malonic esters, malonamide and cyanoacetamide, in which a —CH— or —CH₂— group is situated between. —CO₂R, —CN, or —CONH— groups.

A list of the above compounds can be found in the CRC Handbook—Table for Organic Compound Identification, 3^{rd} Ed. Published by The Chemical Rubber Company, such Table is incorporated herein by reference.

Formulations containing amidoximes may optionally include other complexing agents and the amidoxime compound could have other functional groups that have a chelate functionality within the molecule itself.

The compositions of the present application include semiconductor processing compositions comprising water and at least one compound containing at least one amidoxime functional group. It a preferred embodiment the at least one amidoxime functional groups are derived from a nitrile compound.

In some embodiments the nitrile compound is derived from the cyanoethylation of a compound selected from the group consisting of sugar alcohols, hydroxy acids, sugar acids, monomeric polyols, polyhydric alcohols, glycol ethers, polymeric polyols, polyethylene glycols, polypropylene glycols, amines, amides, imides, amino alcohols, and synthetic polymers.

In use in as a CMP pad cleaner, the cleaning agent may further include one or more oxidizers and one or more surface-active agents, such as a surfactant in the classes disclosed herein (anionic surfactants, Zweitter-ionic surfactants, multi-ionic surfactants, or combinations thereof). Examples of such surfactants include: sodium salts of polyacrylic acid, potassium oleate, sulfosuccinates, sulfosuccinate derivatives, sulfonated amines, sulfonated amides, sulfates of alcohols, alkylanyl sulfonates, carboxylated alcohols, alkylamino propionic acids, alkyliminodipropionic acids, and combinations thereof and wherein the surfactant comprises between about 0.001 to about 10 percent by weight of the composition.

Organic Acid and/or Basic Component

In embodiments of the present invention, the aqueous composition may include: a) a monofunctional, difunctional or trifunctional organic acid; and/or b) one or more basic compounds selected from quaternary amines, hydroxylamine, hydroxylamine derivatives (including salts), hydrazine or hydrazine salt base, ammonium compounds, and one or more alkanolamines.

In another embodiment, the composition contains at least one alkaline (basic) compound that is an alkanolamine. Preferred alkanolamines are monoethanolamine, 2-(2-hydroxylethylamino)ethanol, 2-(2-aminoethoxy)ethanol, N,N,N-tris(2-hydroxyethyl)-ammonia, isopropanolamine, 3-amino-1-propanol, 2-amino-1-propanol, 2-(N-methylamino)ethanol, 2-(2-aminoethylamino)ethanol, and mixtures thereof.

Suitable organic acids include methanesulfonic acid, oxalic acid, lactic acid, citric acid, xylenesulfonic acid, toluenesulfonic acid, formic acid, tartaric acid, propionic acid, benzoic acid, ascorbic acid, gluconic acid, malic acid, malonic acid, succinic acid, gallic acid, butyric acid, trifluoracetic acid, glycolic, and mixtures thereof.

Chelating Agent

In another alternative or additional embodiment, the aqueous composition can include a chelation agent that will complex with transition metal ions and mobile ions. In a preferred embodiment, the chelation agent includes ethylene diamine tetraacetic acid (EDTA), an oxime, 8-hydroxy quinoline, polyalkylenepolyamine or crown ether.

Oxidizing Agent

In another alternative or additional embodiment, the aqueous composition can include an oxidizing agent that will maintain metal film oxide layers. In a preferred embodiment, the oxidizing agent includes ammonium peroxydisulfate, peracetic acid, urea hydroperoxide, sodium percarbonate or sodium perborate. Other oxidizing agents include hydrogen peroxide; hydroxylamine and its salts; nitrate, sulfate, chloride and mixtures; a peracetic acid, perchloric acid, periodic acid and mixtures thereof; persulfates such as ammonium persulfate, sodium persulfate and potassium persulfate, Na₂O₂, Ba₂O₂ and (C₆H₅C)₂O₂; hypochlorous acid (HClO); organic peroxides (ketoneperoxides, diacylperoxides, hydroperoxides, alkylperoxides, peroxyketals, alkylperesters, peroxycarbonates, water-soluble peroxides and such). Among these, hydrogen peroxide (H₂O₂) and hydroxylamine, hydroxylamine sulfate, hydroxyl ammonium salts and mixtures thereof are preferable because they do not contain a metal component or do not generate a harmful byproduct.

The cleaning agents of the current invention include chelation. The cleaning action of the current invention efficiently removes residual particles from the surface of the CMP pad and also complexes the metal that is removed in solution.

The methods of the present invention may also use compositions that are substantially free from fluoride-containing compounds, acid compounds, organic solvents, alkanolamines, quaternary ammonium compounds, hydroxylamine and hydroxylamine derivatives, non-hydroxyl-containing amines, alkanolamines, non-amidoxime group chelating agents, and surfactants.

The compositions herein may contain substantially no additional components.

In some embodiments the organic solvent, which is miscible with water, is in an amount from about 5% to about 15% by weight.

Other preferred embodiments contain a surface active agent, such as: (a) non-ionic; (b) anionic; (c) cationic; (d) zwitterionic; (e) amphoteric surfactants; (f) and mixtures thereof.

In some embodiments, the cleaning agent further comprises a surface-active agent is selected from the group consisting of: (a) non-ionic; (b) anionic; (c) cationic; (d) zwitterionic; (e) amphoteric surfactants; (f) and mixtures thereof and/or at least one basic compound which includes one or more alkanolamines selected from the group consisting of monoethanolamine, 2-(2-hydroxylethylamino)ethanol, 2-(2-aminoethoxy)ethanol, N,N,N-tris(2-hydroxyethyl)-ammonia, isopropanolamine, 3-amino-1-propanol, 2-amino-1-propanol, 2-(N-methylamino)ethanol, 2-(2-aminoethylamino)ethanol, and mixtures thereof in an amount from about 0.5% to about 5% by weight.

It is preferred that the amidoxime group is derived from a nitrile compound that is derived from the cyanoethylation of a compound selected from the group consisting of sugar alcohols, hydroxy acids, sugar acids, monomeric polyols, polyhydric alcohols, glycol ethers, polymeric polyols, polyethylene glycols, polypropylene glycols, amines, amides, imides, amino alcohols, and synthetic polymers.

Examples of amidoximes can be prepared from reacting hydroxylamine with a nitrile compound illustrated in the equation below, for example. Herein a number of amidoxime compounds are disclosed in addition to the example below. Any such compound is for use with the present invention.

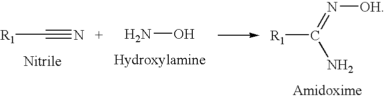

Oxidizing Compound

The oxidizer includes, in some embodiments of the present invention, hydrogen peroxide; hydroxylamine and its salts; nitrate, sulfate, chloride and mixtures; a peracetic acid, perchloric acid, periodic acid and mixtures thereof, persulfates such as ammonium persulfate, sodium persulfate and potassium persulfate, Na₂O₂, Ba₂O₂ and (C₆H₅C)₂O₂; hypochlorous acid (HClO); organic peroxides (ketoneperoxides, diacylperoxides, hydroperoxides, alkylperoxides, peroxyketals, alkylperesters, peroxycarbonates, water-soluble peroxides and such). Among these, hydrogen peroxide (H₂O₂) and hydroxylamine, hydroxylamine sulfate, hydroxyl ammonium salts and mixtures thereof are preferable because they do not contain a metal component or do not generate a harmful byproduct.

A content of the oxidizing agent to the total amount of the CMP pad cleaning composition of the present invention is appropriately set within a range of 0.01 to 10 wt %, taking the polishing efficiency, the polishing accuracy and the like into consideration. The content thereof is set preferably not less than 0.05 wt % and more preferably not less than 0.1 wt % to achieve a better polishing rate, but preferably not greater than 5 wt % and more preferably not greater than 3 wt % to suppress the dishing and regulate the polishing rate. When the content of the oxidizing agent is too low, the chemical effects of the polishing slurry become small so that the polishing rate obtained may become insufficient or the damage may become apparent on the polished face. On the other hand, when the content of the oxidizing agent is too high, its etching capability (chemical effect) against the copper-based metal increases and the dishing is likely to occur.

Additional Complexing Agent

Additionally, pursuant to some embodiments of the present invention, the cleaner may further include other complexing agents for copper, such as such as carboxylic acids and amino acids.

As carboxylic acids, there can be given, for instance, oxalic acid, malonic acid, tartaric acid, malic acid, glutaric acid, citric acid, maleic acid, formic acid, acetic acid, propionic acid, butyric acid, valeric acid, acrylic acid, lactic acid, succinic acid, nicotinic acid and their salts.

As amino acids, there can be given, for instance, arginine, arginine hydrochloride, arginine picrate, arginine flavianate, lysine, lysine hydrochloride, lysine dihydrochloride, lysine picrate, histidine, histidine hydrochloride, histidine dihydrochloride, glutamic acid, sodium glutaminate monohydrate, glutamine, glutathione, glycylglycine, alanine, .beta.-alanine, .gamma.-aminobutyric acid, .epsilon.-aminocarproic acid, aspartic acid, aspartic acid monohydrate, potassium aspartate, calcium aspartate trihydrate, tryptophan, threonine, glycine, cysteine, cysteine hydrochloride monohydrate, oxyproline, isoleucine, leucine, methionine, ornithine hydrochloride, phenylalanine, phenylglycine, proline, serine, tyrosine and valine.

As inorganic acids, there can be given, for instance, nitric acid, nitrous acid, sulfuric acid, sulfurous acid, persulfuric acid, boric acid, perboric acid, phosphoric acid, phosphorous acid, hypophosphorous acid and silicic acid.

An added feature for this invention is to add small quantities of metal ion chelators which could include di-, tri-, tetra-functional groups, i.e., EDTA, citric acid, oximes, lactic acid, 8-hydroxy quinoline and other well known agents that will chelate with metal ions under acid conditions. Other possible agents are polyethylene oxide, polyethyleneimine and crown ethers. These latter two compounds have varying affinity for mobile ions (Li, Na, K, and certain alkaline earth ions). Concentrations preferably vary from 0.01 to 10 wt %.

Surfactants

One preferred cleaning solution of the present invention includes a surface-active agent to promote even wetting of the semiconductor surface. Preferred embodiments include, but are not limited to, non-ionic, anionic, cationic, zwitterionic or amphoteric surfactants or mixtures thereof. Surfactants (nonionics, anionics and cationics) can be included in these formulations.

Other Additives

The cleaning solutions of the present invention may contain a variety of additives such as a dispersing agent, a buffer agent and a viscosity modifier, which are in wide use as common additives to the polishing slurry, provided that they do not affect adversely the properties of the cleaner.

A key component of the formulations of the present invention is the presence of one or more compounds with at least one amidoxime functional group. Without being bound to any particular theory, it is understood that the multidentate complexing agents disclosed above complex with substrate surfaces to remove contaminants on such surfaces. The amidoxime molecule can be designed to function as passivation on metal surface by rendering insoluble metal complex or as cleaning agent by rendering the metal containing residue more soluble.

Amidoxime copper complexes have shown to be readily soluble in water under basic condition while less soluble under acidic condition. Accordingly, the passivating/cleaning effect of the amidoxime chemistry can be affected by altering the pH.

U.S. Pat. No. 6,166,254, for example, discusses the formation of amidoximes from aqueous hydroxylamine freebase and nitriles, such as the reaction of acetonitrile with aqueous hydroxylamine at ambient temperature to yield high purity acetamidoxime.

It will be obvious to those skills of the art that other nitriles will react with hydroxylamine freebase in similar manners.

Amidoximes have been shown to complex with metals, such as copper. Amidoximes of cyanoethylated cellulose have also been shown to complex with copper and other metal ions. (See, Altas H. Basta, International Journal of Polymeric Materials, 42, 1-26 (1998)).

One preferred embodiment of the present invention is to compositions, and methods of use thereof, containing a group of higher pH range chelating compounds comprising at least two functional groups where at least one such group is an amidoxime. The other groups or complexing compounds may be selected as may be beneficial for the application, the chemistry, and/or the conditions. Examples of other complexing groups include hydroxamic acid, thiohydroxamic acid, N-hydroxyurea, N-hydroxycarbamate, and N-nitroso-alkyl-hydroxylamine. These groups offer synergistic advantages when used with amidoximes of removing metal oxide, such as copper oxide, residue by rendering such oxides soluble in aqueous solutions. As with amidoximes, these functional groups can be formed by reaction with hydroxylamine or hydroxylamine derivatives.

Regarding other complexing agents that may optionally be used with amidoximes in the compositions of the present application, complexing agents may be purchased commercially or prepared by known methods. A non-exhaustive list has been previously presented.

One example of a synergistic functional group is a hydroxamic acid group. Such groups are well known (H. L. Yale, “The Hydroxamic Acids”, Chem. Rev., 209-256 (1943)). Polymers containing hydroxamic acid groups are known and can be prepared by addition of hydroxylamine to anhydride groups of anhydride-containing copolymers, such as styrene-maleic anhydride copolymer or poly(vinylmethylether/maleic anhydride) copolymers, or by reaction of hydroxylamine with ester groups. Hydroxamic acid-containing polymers can also be prepared by acid-catalyzed hydrolysis of polymers that contain amidoxime groups (U.S. Pat. No. 3,345,344).

U.S. Pat. No. 6,235,935, for example, discusses the formation of high purity oximes from aqueous hydroxylamine and ketones reacted at ambient temperature without addition of impurities such as salts or acids.

Thiohydroxamic acids are compounds with another synergistic type of functional group with amidoximes and can be prepared by addition of hydroxylamine to dithiocarboxylic acids (H. L. Yale, Chem. Rev., 33, 209-256 (1943)).

N-hydroxyureas are compounds with another synergistic type of functional groups with amidoximes and can be prepared by reaction of hydroxylamine with an isocyanate (A. O. Ilvespaa et al., Chime (Switz.) 18, 1-16 (1964)).

N-Hydroxycarbamates are compounds with another synergistic type of functional groups with amidoximes and can be prepared by reaction of hydroxylamine with either a linear or cyclic carbonate (A. O. Ilvespaa et al., Chimia (Switz.) 18, 1-16 (1964)).

N-Nitroso-alkyl-hydroxylamines are compounds with another synergistic type of functional group with amidoximes and can be prepared by nitrosation of alkyl hydroxylamines (M. Shiino et al., Bioorganic and Medicinal Chemistry 95, 1233-1240 (2001)).

One embodiment of the present invention involves cleaning solutions which comprise at least one chelating compound with one or more amidoxime functional group.

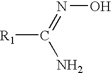

The amidoximes can be prepared by the reaction of nitrile-containing compounds with hydroxylamine.

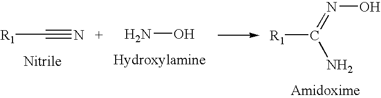

A convenient route to the formation of amidoxime chelating compounds is by adding hydroxylamine to the corresponding nitrile compound. There are several methods known for preparing nitrile-containing compounds, including cyanide addition reactions such as hydrocyanation, polymerization of nitrile-containing monomers to form polyacrylonitrile or copolymers of acrylonitrile with vinyl monomers, and dehydration of amides. Typical procedures for the syntheses of nitriles may be found in J. March, Advanced Organic Chemistry, 4th ed., John Wiley and Sons, NY, (1992).

Nitrile compounds listed in the CRC Handbook (pages 344-368) can be used in this invention include, but are not limited to, the following: Cyanoacetylene, Cyanoacetaldehyde. Acrylonitrile, Fluoroacetonitrile, Acetonitrile (or Cyanomethane), Trichloroacetonitrile, Methacrylonitrile (or α-Methylacrylonitrile), Proionitrile (or Cyanoethane), Isobutyronitrile, Trimethylacetonitrile (or tert-Butylcyanide), 2-Ethyacrylonitrile, Dichloroacetonitrile, α Chloroisobutyronitrile, n-Butyronitrile (or 1-Cyanopropane), trans-Crotononitrile, Allycyanide, Methoxyacetonitrile, 2-Hydroxyisobutyronitrile (or Acetone cyanohydrins), 3-Hydroxy-4-methoxybenzonitrile, 2-Methylbutyronitrile, Chloroacetonitrile, Isovaleronitrile, 2,4-Pentadienonitrile, 2-Chlorocrotononitrile, Ethoxyacetonitrile, 2-Methycrotononitrile, 2-Bromoisobutyronitrile, 4-Pentenonitrile, Thiophene-2,3-dicarbonitrile (or 2,3-Dicyanothiophene), 3,3-Dimethylacrylonitrile, Valeronitrile (or 1 Cyanobutane), 2-Chlorobutyronitrile, Diethylacetonitrile, 2-Furanecarbonitrile (or beta-Furonitrile; 2 Cyanofuran), 2-Methylacetoacetonitrile, Cyclobutanecarbonitrile (or Cyanocyclobutane), 2-Chloro-3-methylbutyronitrile, Isocapronitrile (or 4-Methylpentanonitrile), 2,2-Dimethylacetoacetonitrile, 2-Methylhexanonitrile, 3-Methoxypropionitrile, n-Capronitrile (n-Hexanonitrile), (Ethylamino) acetonitrile (or N-Ethylglycinonitrile), d,l-3-Methylhexanonitrile, Chlorofumaronitrile, 2-Acetoxypropionitrile (or O-Acetyllactonitrile), 3-Ethoxypropionitrile, 3-Chlorobutyronitrile, 3-Chloropropionitrile, Indole-3-carbonitrile (or 3-Cyanoindole), 5-Methylhexanonitrile, Thiophene-3-carbonitrile (or 3-Cyanothiophene), d,l-4-Methylhexanonitrile, d,l-Lactonitrile (or Acetaldehydecyanohydrin), Glycolnitrile (or Formaldehydecyanohydrin), Heptanonitrile, 4-Cyanoheptane, Benzonitrile, Thiophene-2-carbonitrile (or 2-Cyanothiophene), 2-Octynonitrile, 4-Chlorobutyronitrile, Methyl cyanoacetate, Dibenzylacetonitrile, 2-Tolunitrile (or 2-Methoxybenzonitrile), 2,3,3-Trimethyl-1-cyclopentene-1-carbonitrile (or □-Campholytonitrile), Caprylonitrile (or Octanonitrile), 1,1-Dicyanopropane (or Ethylmalononitrile), Ethyl cyanoacetate, 1,1-Dicyanobutane (or Propylmalononitrile), 3-Tolunitrile (or 3-Methylbenzonitrile), Cyclohexylacetonitrile, 4,4-Dicyano-1-butene (or Allylmalononitrile), 3-Isopropylidene-1-methyl-cyclopentane-1-carbonitrile (or β Fencholenonitrile), 3-Hydroxypropionitrile, 1,1-Dicyano-3-methylbutane (or Isobutylmalononitrile), Nonanonitrile, 2-Phenylcrotononitrile, Ethylenecyanohydrin, 2-Phenylpropionitrile, Phenylacetonitrile (or Benzylcyanide), Phenoxyacetonitrile, 4-Hydroxy-butyronitrile, (3-Tolyl)acetonitrile (or m-Xylycyanide), (4-Tolyl)acetonitrile (or p-Xylycyanide), 4-Isopropylbenzonitrile, (2-Tolyl)acetonitrile (or o-Xylycyanide), Decanonitrile, 3-Methyl-2-phenylbutyronitrile, 1,2-Dicyanopropane, 1-Undecanonitrile (or 1-Hendecanonitrile), 2-Phenylvaleronitrile, 10-Undecenonitrile (or 10 Hendecenonitrile), 3-Phenylpropionitrile, 2-Cyanobenzalchloride (or α,α Dichloro-o-tolunitrile), N-Methylanilinonitrile (or N-Cyano-N-methylaniline), 3-(2-Chlorophenyl)propionitrile, 1,3-Dicyano-2-methypropane (or 2-Methylglutaronitrile), O-Benzoyl lactonitrile (or Lactonitrile benzoate), 3-Cyanobenzalchloride (or α,α-Dichloro-m-tolunitrile), 4-Cyanobenzalchloride (or α,α-Dichloro-p-tolunitrile), Dodecanonitrile (or Lauronitrile), 1,3-Dicyanopropane (or Glutaronitrile), 4-Methoxyhydrocinnamonitrile (or 3-(4-Methoxyphenyl)-propionitrile), 1,4-Dicyanobutane (Adiponitrile), 1,2,2,3-Tetramethyl-3-cyclopentene-1-acetonitrile (or 5-Methyl-α-campholenonitrile), 1-Cyanocyclohexene, 2-Hydroxybutyronitrile (or Propanalcyanohydrin), Hydnocarponitrile, α-Chloro-α-phenylacetonitrile, Butyl cyanoacetate, 3-Bromopropionitrile, 2,4-Diphenylbutyronitrile, Thiophene-2-acetonitrile, Trans-4-Chlrocrotononitrile, 2-Cyanopentanoic acid, Azelaonitrile (or 1,7-Dicyanoheptane), 3-Chloro-2-hydroxy-2-methylpropionitrile (or Chloroacetone cyanohydrins), 1,11-Dicyanoundecane (or 1,11-Dicyanohendecane), 2-Cyanobutyric acid, 2-Cyanobiphenyl, 1,12-Dicyanodedecane (or α,ω-Dodecane dicyanide), 1-Cyano-4-isopropenylcyclohexene, Sebaconitrile (or 1,8-Dicyanooctane), Suberonitrile (or 1,6-Dicyanohexane), 3-Cyanoindene (or indene-3-carbonitrile), Aminoacetonitrile (or Glycinonitrile), 2-Cyanodiphenylmethane, N-Piperidinoacetonitrile, 3-Chloro-2-tolunitrile, Tetradecanonitrile, Cinnamonitrile, Trichloroacrylonitrile, DL-Mandelonitrile (or Benzaldehyde cyanohydrins), Pentadecanonitrile, 2-Methoxybenzonitrile, (2-Chlorophenyl)acetonitrile (or 2-Chlorobenzylcyanide), 1,1-Dicyanoethane (or Methylmalononitrile), 2-Cyanopyridine (or 2-Pyridinecarbonitrile; Picolinonitrile), 4-tolunitrile (or 4-Methylbenzonitrile), D-Mandelonitrile, d,l-(2-Bromophenyl)acetonitrile (or 2-Bromobenzyl cyanide), (4-Chlorophenyl)acetonitrile (or 4-Chlorobenzyl cyanide), Malononitrile (or Methylene cyanide), Hexadecanonitrile, Maleonitrile (or cis-1,2-Dicyanoethylene), 2,2-Dicyanopropane (or Dimethylmalononitrile), tert-Butylacetonitrile (or Neopentyl cyanide), 1-Naphthylacetonitrile, 4,4-Dicyanoheptane (or Dipropylmalononitrile), Heptadecanonitrile, 1-Naphthonitrile (or 1-Cyanonapthalene), 2-Cyanopropionic acid, 4-Fluorobenzonitrile, Coumarilonitrile (or Coumarin-2-carbonitrile), Indole-3-acetonitrile, 3-Bromobenzonitrile, 2-(N-Anilino)-butyronitrile, Trans-o-Chlorocinnamonitrile, Octadecanonitrile, 3-Chlorobenzonitrile, 2-Chlorobenzonitrile, 4-Chloromandelonitrile, Nonadecanonitrile, 2-Bromo-4-tolunitrile, 3,3-Dicyanopentane (or Diethylmalononitrile), 4-Cyanobutyric acid, 5-Chloro-2-tolunitrile, (4-Aminophenyl)acetonitrile (or 4-Aminobenzyl cyanide), meso-2,3-Dimethyl-succinonitrile, 3-Bromo-4-tolunitrile, (4-Bromophenyl)acetonitrile (or 4-Bromobenzyl cyanide), N-Anilinoacetonitrile, 3-Cyanopropionic acid, 3-Chloro-4-tolunitrile, 3,3-Diphenylacrylonitrile (β-Phenylcinnamonitrile), 3-Bromo-2-hydroxy benzonitrile, 4,4-Dicyanoheptane (or Dipropylmalononitrile), trans-2,3-Diphenyl acrylonitrile, Eicosanonitrile, 3-Cyanopyridine (or Nicotinonitrile), (4-Iodophenyl)acetonitrile (or 4-Iodobenzyl cyanide), 4-Cyanodiphenyl methane, 2-(N-Anilino) valeronitrile, 2-Aminobenzonitrile (or Anthranilonitrile), 2-Bromobenzonitrile, 5-Cyanothiazole, 3-Aminobenzonitrile, 2-Quinolinoacetonitrile, 2-Iodobenzonitrile, 2,4,6-Trimethylbenzonitrile, α-Aminobenzyl cyanide, Cyanoform (or Tricyanomethane), Succinonitrile, 2-Iodo-4-tolunitrile (2-Iodo-4-methylbenzonitrile), 2,6-Dinitrobenzonitril, d,l-2,3-Dimethylsuccinonitrile, 2-Chloro-4-tolunitrile, 4-Methoxybenzonitrile, 2,4-Dichlorobenzonitrile, 4-Methoxycinnamonitrile, 3,5-Dichlorobenzonitrile, cis-1,4-Dicyanocyclohexane, Bromomalononitrile, 2-Naphthonitrile (or 2-Cyanonaphthalene), Cyanoacetic acid, 2-Cyano-2-ethylbutyric acid (or Diethylcyanoacetic acid), 2,4-Diphenylglutaronitrile, beta-Chloro-3-tolunitrile, 4-Chloro-2-tolunitrile, 1-Cyanoacenaphthene (or Acenaphthene-1-carbonitrile), Phenylmalononitrile (β-Cyanobenzyl cyanide), 6-Nitro-2-tolunitrile, (4-Hydroxyphenyl)acetonitrile (or 4-Hydroxybenzyl cyanide), 5-Bromo-2-tolunitrile, β-Bromo-2-tolunitrile, 2,2-Diphenylglutaronitrile, (2-Aminophenyl)acetonitrile (or 2-Aminobenzyl cyanide), 3,4-Dichlorobenzonitrile, 1,2,2,3-Tetramethylcyclopentene-1-carbonitrile (or Campholic nitrile), Dicyanodimethylamine (or Bis(cyanomethyl) amine), Diphenylacetonitrile (β-Phenylbenzyl cyanide), 4-Cyano-N,N-dimethylaniline, 1-Cyanoisoquinoline, 4-Cyanopyridine, β-Chloro-4-tolunitrile (or 4-Cyanobenzyl chloride), 2,5-Diphenylvaleronitrile, 3-Cyanobenzaldehyde (or 3-Formylbenzonitrile), 6-Nitro-3-tolunitrile, Benzoylacetonitrile, 6-Chloro-2-tolunitrile, 8-Cyanoquinoline, 2-Nitro-3-tolunitrile, 2,3,4,5-Tetrachlorobenzonitrile, 4-Cyanobiphenyl, 2-Naphthylacetonitrile, cis-2,3-Diphenylacrylonitrile, 4-Aminobenzonitrile (or 4-Cyanoaniline), 1-Cyano-2-phenylacrylonitrile (or Benzalmalononitrile), 5-Bromo-2,4-dimethyl-benzonitrile, 2-Cyanotriphenylmethane, 5-Cyanoquinoline, 2,6-Dimethylbenzonitrile, Phenylcyanoacetic acid, 2-(N-Anilino)-propionitrile, 2,4-Dibromobenzonitrile, β-(2-Nitrophenyl)-acrylonitrile, 5-Chloro-2-nitro-4-tolunitrile, βBromo-3-tolunitrile (or 3-Cyanobenzyl bromide), 4-Nitro-3-tolunitrile, 2-(N-Anilino)-isobutyronitrile, 2-Cyanoquinoline, 4-Cyanovaleric acid (or 2-Methylglutaromononitrile), Fumaronitrile, 4-Chlorobeuzonitrile, 9-Phenanthrylacetonitrile, 3,5-Dibromobenzonitrile, 2-Chloro-3-nitrobenzonitrile, 2-Hydroxybenzonitrile (or 2-Cyanophenol), 4-Chloro-2-nitrobenzonitrile, 4-Cyanotriphenylmethane, 4-Chloro-3-nitrobenzonitrile, 3-Nitro-4-tolunitrile, 2-Cyano-3-phenylpropionic acid, 3-Cyanophenanthrene, 2,3,3-Triphenylpropionitrile, 4-Cyanoquinoline, 4-Bromo-1-naphthonitrile (or 1-Bromo-4-cyanonaphthalene), 4-Bromo-2,5-dimethylbenzonitrile, 5-Nitro-3-tolunitrile, 2,4-Dinitrobenzonitrile, 4-Nitro-2-tolunitrile, 6-Chloro-3-nitrobenzonitrile, 5-Bromo-3-nitro-2-tolunitrile, 2-Nitro-4-tolunitrile, 9-Cyanophenanthrene, 3-Cyanoquinoline, 2-Cyanophenanthrene, 3-Nitro-2-tolunitrile, 2-Nitrobenzonitrile, 4-Chloro-1-naphthonitrile (or 1-Chloro-4-cyanonaphthalene), 5-Cyanoacenaphthene (or Acenaphthene-5-carbonitrile), 4-Bromobenzonitrile, 2,4,5-Trimethoxybenzonitrile, 4-Hydroxybenzonitrile (or 4-Cyanophenol), 2,3-Diphenylvaleronitrile, βBromo-4-tolunitrile (or 4-Cyanobenzylbromide), (4-Nitrophenyl)aceto nitrile (or 4-Nitrobenzylcyanide), 6-Bromo-3-nitrobenzonitrile, (2-Hydroxyphenyl)acetonitrile (or 2-Hydroxybenzyl cyanide), 3-Nitrobenzonitrile, 4-Bromo-3-nitrobenzonitrile, 4-Cyanoazobenzene, Dipicolinonitrile (or 2,6-Dicyanopyridine), 2-Cyanohexanoic acid, Dibrornomalononitrile (or Bromodicyanomethane), 1-Cyanoanthracene, 2,2,3-Triphenylpropionitrile, 1-Cyanophenanthrene, 2,3-Diphenylbutyronitrile, 5-Bromo-3nitro-4-tolunitrile, 2,5-Dichlorobenzonitrile, 2,5-Dibromobenzonitrile, 5-Bromo-2-nitro-4-tolunitrile, 2-Hydroxy-3-nitrobenzonitrile (or 2-Cyano-6-nitrophenol), 4-Nitro-1-naphthonitrile (or 1-Cyano-4-nitronaphthalene), 4-Acetamidobenzonitrile, 6-Cyanoquinoline, Apiolonitrile (or 2,5-Dimethoxy-3,4-methylenedioxybenzonitrile), 1-Nitro-2-naphthonitrile (or 2-Cyano-1-nitronaphthalene), 3,5-Dichloro-2-hydroxyhenzonitrile, trans-1,4-Dicyanocyclohexane, 3,3,3-Triphenylpropionitrile, 4-Cyano-2-phenylquinoline (or 2-Phenyl-4quinolinonitrile), Phthalonitrile (or o-Dicyanobenzene), 8-Nitro-2-naphthonitrile (or 2-Cyano-8-nitronaphthalene), 5-Chloro-2-naphthonitrile (or 5-Chloro-2cyanonaphthalene), 5-Chloro-1-naphthonitrile (or 5-Chloro-1-cyanonaphthalene), 3,5-Dichloro-4-hydroxybenzonitrile, 4-Nitrobenzonitrile, 5-Bromo-1-naphthonitrile (or 1-Bromo-5cyanonaphthalene), 5-Iodo-2-naphthonitrile (or 2-Cyano-5-iodonaphthalene), 3-Cyano-3-phenylpropionic Acid, 2-Cyano-2-propylvaleramide (or Dipropylcyanoacetamide), 2,6-Dibromobenzonitrile, 3-Chloro-4-hydroxybenzonitrile, 5-Chloro-2,4-dinitrobenzonitrile, 4-Benzamidobenzonitrile (or N-Benzoylanthranilonitrile), 5-Bromo-2-hydroxybenzonitrile, d,l-2,3-Diphenylsuccinonitrile, Isophthalonitrile (or m-Dicyanobenzene), 2-Hydroxy-4-nitrohenzonitrile (or 2-Cyano-5-nitrophenol), d,l-4-Cyano-3,4-diphenylbutyric acid (or d,l-2,3-Diphenylglutaromononitrile), d-3-Carboxy-2,2,3-trimethyicyclopentylacetonitrile, 5-Chloro-2-hydroxyhenzonitrile (or 4-Chloro-2-cyanophenol), 2,3-Diphenylcinnamonitrile (or Cyanotriphenylethylene), 1,7-Dicyanonaphthalene, 4,4′-Dicyanodiphenylmethane, 2,2′-Diphenic acid mononitrile (or 2-Carboxy-2′-cyanobiphenyl), 5-Nitro-2-naphthonitrile (or 2-Cyano-5-nitronaphthalene), 9-Cyanoanthracene (or 9-Anthracenecarbonitrile), 2,3-Dicyanopyridine, 1,3-Dicyanonaphthalene, 3-Cyanocoumarin, 2-Cyanocinnamic acid, 2-Cyanobenzoic acid, 1,2-Dicyanonaphthalene, 2-Hydroxy-5-nitrobenzonitrile (or 2-Cyano-4-nitrophenol), Tetracyanoethylene, 5-Nitro-1-naphthonitrile (or 1-Cyano-5-nitronaphthalene), 1,4-Dicyanonaphthalene, 1,6-Dicyanonaphthalene, 1,5-Dicyanonaphthalene, 3-Cyanobenzoic acid, 4-Cyanobenzoic acid, Terephthalonitrile (or p-Dicyanobenzene), 1,8-Dicyanonaphthalene, 4,4′-Dicyanobiphenyl, 1-2,3-Diphenylsuccinonitrile, 1-Cyano-9,10-anthraquinone, 2,3-Dicyanonaphthalene, 2,7-Dicyanonaphthalene, 2,6-Dicyanonaphthalene.

The present invention further includes the “nitrile quaternaries”, cationic nitrites of the formula

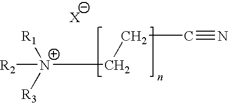

in which R₁ is —H, —CH₃, a C₂₋₂₄-alkyl or -alkenyl radical, a substituted C₂₋₂₄-alkyl or -alkenyl radical with at least one substituent from the group —Cl, —Br, —OH, —NH₂, —CN, an alkyl- or alkenylaryl radical with a C₁₋₂₄-alkyl group, or is a substituted alkyl- or alkenylaryl radical with a C₁₋₂₄-alkyl group and at least one further substituent on the aromatic ring, R₂ and R₃, independently of one another, are chosen from CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH where n−1, 2, 3, 4, 5 or 6 and X is an anion.

The general formula covers a large number of cationic nitrites which can be used within the scope of the present invention. With particular advantage, the detergent and cleaner according to the invention comprise cationic nitrites in which R₁ is methyl, ethyl, propyl, isopropyl or an n-butyl, n-hexyl, n-octyl, n-decyl, n-dodecyl, n-tetradecyl, nhexadecyl or n-octadecyl radical. R₂ and R₃ are preferably chosen from methyl, ethyl, propyl, isopropyl and hydroxyethyl, where one or both of the radicals may advantageously also be a cyanomethylene radical.

For reasons of easier synthesis, preference is given to compounds in which the radicals R₁ to R₃ are identical, for example (CH₃)₃N⁽⁺⁾CH₂—CN (X⁻), (CH₃CH₂)₃N⁽⁺⁾CH₂—CN X⁻, (CH₃CH₂CH₂)₃N⁽⁺⁾CH₂—CN X⁻, (CH₃CH(CH₃))₃N⁽⁺⁾CH₂—CN X⁻ or (HO—CH₂—CH₂)₃N⁽⁺⁾CH₂—CN X⁻, where X⁻ is preferably an anion which is chosen from the group consisting of hydroxide, chloride, bromide, iodide, hydrogensulfate, methosulfate, p-toluenesulfonate (tosylate) or xylenesulfonate.

Examples of typical acrylonitrile polymeric materials, which serve as precursors for preparing our polyamidoximes, are listed below. The figures are the percents by weight of each monomer in the polymer.

| | | |
|---|---|---|
| 90% acrylonitrile | 10% vinylacetonitrile | |
| 50%′ acrylonitrile | 50% methacrylonitrile | |
| 97% acrylonitrile | 3% vinyl acetate | |
| 50% acrylonitrile | 50% vinyl acetate | |
| 95% acrylonitrile | 5% methyl methacrylate | |
| 65% acrylonitrile | 35% methyl acrylate | |
| 45% acrylonitrile | 10% methyl acrylate | 45% vinyl acetate |
| 44% acrylonitrile | 44% vinyl chloride | 12% methyl acrylate |
| 93% acrylonitrile | 7% 2-vinyl pyridine | |
| 26% acrylonitrile | 74% butadiene | |
| 40% 1 acrylonitrile | 60% butadiene | |
| 33% acrylonitrile | 67% styrene | |

100% acrylonitrile

Several of the polymers are available commercially, such as:

| Product | Manufacturer | Composition |
|---|---|---|
| Orion | DuPont de Nemours | 90% Acrylonitriles |
| Acrilan | Chemstrand | 90% Acrylonitriles |
| Creslan | American Cyanamid | 95-96% Acrylonitriles |
| Zefran | Dow Chemical Co. | 90% Acrylonitriles |
| Verel | Eastman | About 50% acrylonitrile |
| Dyrel | Carbide &Carbon | 40% acrylonitrile-60% Vinyl chloride |
| | Chemical | |
| Darlan | B. F Goodrich | 50 Mole percent vinylidene cyanide - |
| | | 50 Mole percent Vinyl acetate |

A particularly useful route to nitrites is termed “cyanoethylation”, in which acrylonitrile undergoes a conjugate addition reaction with protic nucleophiles such as alcohols and amines. Other unsaturated nitrites can also be used in place of acrylonitrile.

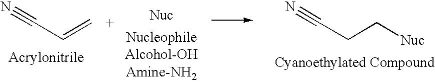

Preferred amines for the cyanoethylation reaction are primary amines and secondary amines having 1 to 30 carbon atoms, and polyethylene amine. Alcohols can be primary, secondary, or tertiary. The cyanoethylation reaction (or “cyanoalkylation” using an unsaturated nitrile other than acrylonitrile) is preferably carried out in the presence of a cyanoethylation catalyst. Preferred cyanoethylation catalysts include lithium hydroxide, sodium hydroxide, potassium hydroxide and metal ion free bases from tetraalkylammonium hydroxide, such as tetramethylammonium hydroxide, TMAH pentahydrate, BTMAH (benzyltetramethylammonium hydroxide), TBAH, choline, and TEMAH (Tris(2-hydroxyethyl)methylammonium hydroxide). The amount of catalyst used is typically between 0.05 mol % and 15 mol %, based on unsaturated nitrile.

Preferably, the cyanolates are derived from the following groups: arabitol, erythritol, glycerol, isomalt, lactitol, maltitol, mannitol, sorbitol, xylitol, sucrose and hydrogenated starch hydrosylate (HSH).

The hydroxy acids can include but are not limited to the following: hydroxyphenylacetic acid (mandelic acid), 2-hydroxypropionic acid (lactic acid), glycolic acid, hydroxysuccinic acid (malic acid), 2,3-dihydroxybutanedioic, acid (tartaric acid), 2-hydroxy-1,2,3-propanetricarboxylic, acid (citric acid), ascorbic acid, 2-hydroxybenzoic, acid (salicylic acid), 3,4,5-trihydroxybenzoic acid (gallic acid).

The sugar acids can include but are not limited to the following: galactonic acid, mannonic, acid, fructonic acid, arabinonic acid, xylonic acid, ribonic, acid, 2-deoxyribonic acid, and alginic acid.

The amino acids can include but are not limited to the following: alanine, valine, leucine, isoleucine, proline, tryptophan, phenylalanine, methionine, glycine, serine, tyrosine, threonine, cysteine, asparagine, glutamine, aspartic acid, glutamic acid, lysine, arginine, and histidine.

The group of monomeric polyols- or polyhydric alcohols, or glycol ethers, can be chosen from ethanol, n- or isopropanol, butanols, glycol, propane- or butanediol, glycerol, diglycol, propyl or butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl, ethyl or propyl ether, dipropylene glycol methyl or ethyl ether, methoxy, ethoxy or butoxy triglycol, 1-butoxyethoxy-2-propanol, 3-methyl-3-methoxybutanol, propylene glycol t-butyl ether, and pentaerythritol.

The group of polymeric polyols can be chosen from the group of polyethylene glycols and polypropylene glycols:

Polyethylene glycols (abbreviation PEGS) PEGs are polymers of ethylene glycol which satisfy the general formula

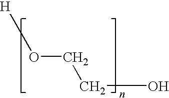

where n can assume values between 1 (ethylene glycol, see below) and about 16. Polyethylene glycols are commercially available, for example under the trade names Carbowax® PEG 200 (Union Carbide), Emkapol® 200 (ICT Americas), Lipoxol® 200 MED (HOLS America), Polyglycol E-200 (Dow Chemical), Alkapol® PEG 300 (Rhone-Poulenc), Lutrol® E300 (BASF), and the corresponding trade names with higher numbers.

Polypropylene glycols (PPGs) which can be used according to the invention are polymers of propylene glycol which satisfy the general formula

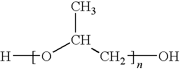

where n can assume values between 1 (propylene glycol) and about 12. Of industrial significance here are, in particular, di-, tri- and tetrapropylene glycol, i.e. the representatives where n=2, 3 and 4 in the above formula.

From the group of organic nitrogen compounds:

Amines: Amines are organic compounds and a type of functional group that contain nitrogen as the key atom. Structurally amines resemble ammonia, wherein one or more hydrogen atoms are replaced by organic substituents such as alkyl, aryl and cyclic groups. Compounds containing one or more —NH— groups of the formula:

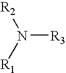

Amides—an amide is an amine where one of the nitrogen substituents is an acyl group; it is generally represented by the formula: R₁(CO)NR₂R₃, where either or both R₂ and R₃ may be hydrogen. Specifically, an amide can also be regarded as a derivative of a carboxylic acid in which the hydroxyl group has been replaced by an amine or ammonia, in which a —CH— or —CH₂— group is situated between —CONH— groups.

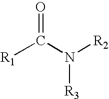

Imides—imide is a functional group consisting of two carbonyl groups bound to a primary amine or ammonia. The structure of the imide moiety is as shown, which possessing a —CH—, —CH₂—, or —CH₃ group adjacent to the carbonyl group.

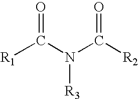

From the group of amino alcohol (or alkanolamine)—Amino alcohols are organic compounds that contain both an amine functional group and an alcohol functional, where the amine can be primary or secondary amines of the formula, wherein X is independently selected from alkylene, heteroalkylene, arylene, heteroarylene, alkylene-heteroaryl, or alkylene-aryl group.

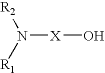

From the group of synthetic polymers: Synthetic polymers such as acetone-formaldehyde condensate, acetone-isobutyraldehyde condensate, methyl ethyl ketone-formaldehyde condensate, poly(allyl alcohol), poly(crotyl alcohol), poly(3-chloroallyl alcohol), ethylene-carbon monoxide copolymers, polyketone from propylene, ethylene and carbon monoxide, poly(methallyl alcohol, poly(methyl vinyl ketone, and poly(vinyl alcohol).

Synthetic polymers such as acetone-formaldehyde condensate, acetone-isobutyraldehyde condensate, methyl ethyl ketone-formaldehyde condensate, poly(allyl alcohol), poly(crotyl alcohol), poly(3-chloroallyl alcohol), ethylene-carbon monoxide copolymers, polyketone from propylene, ethylene and carbon monoxide, poly(methallyl alcohol, poly(methyl vinyl ketone, and poly(vinyl alcohol) have also been cyanoethylated and can also serve as platforms for further modification into metal-binding polymers.

The nitrile groups of these cyanoethylates or cyanoalkylates can be reacted with hydroxylamine to form the amidoxime. In the process described herein for preparing amidoxime groups, hydroxylamine, hydroxylamine hydrochloride, and hydroxylamine sulfate are suitable sources of hydroxylamine. If hydroxylamine salt is used instead of hydroxylamine freebase, a base such as sodium hydroxide, sodium carbonate or metal ion free base such ammonium hydroxide, tetraalkylammonium hydroxide should be used to release hydroxylamine as freebase for the reaction.

Metal ion freebase, such as ammonium hydroxide or a group of tetraalkylammonium hydroxide, such as tetramethylammonium hydroxide, TMAH pentahydrate, BTMAH (benzyltetramethylammonium hydroxide), TBAH, choline, and TEMAH (Tris(2-hydroxyethyl)methylammonium hydroxide) are preferred.

Metals, such as copper and others, complex strongly with molecules containing amidoxime groups, for example amidoximes of sucrose and sorbitol, to bind metal contaminant residues.

The present invention offers the benefit of binding to the metal oxide surface to create an oxidation barrier, particularly where the amidoxime is derived from functionalized amidoxime polymer, such as from polyvinylalcohol, polyacrylonitriles and its copolymers.

The present invention utilizes the cyanoethylated compounds referenced in “The Chemistry of Acrylonitrile, 2nd ed.” as starting materials for synthesis of amidoximes, such reference is incorporated herein to the extent of the cyanoethylated compounds disclosed therein. The most preferred staring materials for synthesis of amidoximes are those prepared from cyanoethylated sugar alcohols, like sucrose, or reduced sugar alcohols, like sorbitol.

The present invention further offers the benefit of increasing the bulk removal of metal during the CMP process when a chelating agent disclosed herein (e.g., (1,2,3,4,5,6-(hexa-(2-amidoximo)ethoxy)hexane) combined with a compound with oxidation and reduction potentials such as hydroxylamine and its salts, hydrogen peroxide, hydrazines.

Because the chelating agents disclosed herein are not carboxylic acid based but are instead contain multiple ligand sites, the present invention further offers the benefit of more efficient and effective binding to metal ions found in semiconductor manufacturing processes, such as residue after plasma etching particularly with leading edge technology where copper is used as conducting metal.

Another advantage of the chelating agents disclosed herein is that such chelating agent could be used in dilution as a Post-copper CMP clean because these groups of compounds are less acidic than organic acid and less basic than ammonia, choline hydroxide and THEMAH.

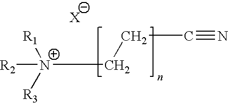

### General Procedures on Preparation of Amidoxime

Examples of cyanoethylation to produce nitrile compounds:

### Preparation of β-Ethoxypropionitrile, C₂H₅—O—CH₂—CH₂—CN

Place 25 ml of 2 percent aqueous sodium hydroxide and 26 g (33 ml) of ethyl alcohol in a 250 ml. reagent bottle, add 26.5 g (33 ml) of acrylonitrile and close the mouth of the bottle with a tightly-fitting cork. Shake the resulting clear homogeneous liquid in a shaking machine for 2 hours. During the first 15 minutes the temperature of the mixture rises 15° to 20° and thereafter falls gradually to room temperature; two liquid layers separate after about 10 minutes. Remove the upper layer and add small quantities of 5 percent acetic acid to it until neutral to litmus; discard the lower aqueous layer. Dry with anhydrous magnesium sulfate, distil and collect the β-Ethoxypropionitrile at 172-1740. The yield is 32 g.

### β-n-Propoxypropionitrile, C₃H₇—O—CH₂—CH₂—CN

Introduce 0.15 g of potassium hydroxide and 33 g. (41 ml) of dry n-propyl alcohol into a 150 ml. bolt-head flask, warm gently until the solid dissolves, and then cool to room temperature. Clamp the neck of the flask and equip it with a dropping funnel, a mechanical stirrer and a thermometer (suitably supported in clamps). Introduce from the dropping funnel, with stirring, 26.5 g. (33 ml) of pure acrylonitrile over a period of 2.5-30 minutes (1 drop every ca. 2 seconds). Do not allow the temperature of the mixture to rise above 35-45°, immerse the reaction flask in a cold water bath, when necessary. When all the acrylonitrile has been added, heat under reflux in a boiling water bath for 1 hour; the mixture darkens. Cool, filter and distil. Collect the O-n-Propoxypropionitrile at 187-189°. The yield is 38 g.

### β-Diethylaminopropionitrile, (C₂H₅)₂N—CH₂—CH₂—CN

Mix 42.5 g (60 ml) of freshly-distilled diethylamine and 26.5 g. (33 ml) of pure acrylonitrile in a 250 ml round-bottomed flask fitted with a reflux condenser. Heat at 50° in a water bath for 10 hours and then allow to stand at room temperature for 2 days. Distil off the excess of diethylamine on a water bath, and distil the residue from a Claisen flask under reduced pressure. Collect the β-Diethylaminopropionitrile at 75-77°/11 mm. The yield is 54 g.

### β-Di-n-butylaminopropionitrile, (C₄H₉)₂N—CH₂—CH₂—CN

Proceed as for the diethyl compound using 64.5 g. (85 ml) of redistilled di-n-butylamine and 26.5 g. (33 mL) of pure acrylonitrile. After heating at 50° and standing for 2 days, distil the entire product under diminished pressure (air bath); discard the low boiling point fraction containing unchanged di-n-butylamine and collect the β-Di-n-butylaminopropionitrile at 120-122°110 mm. The yield is 55 g.

### Ethyl n-propyl-2-cyanoethylmalonate

Add 8.0 g (10.0 ml) of redistilled acrylonitrile to a stirred solution of ethyl n-propyl malonate (30.2 g.) and of 30 percent methanolic potassium hydroxide (4.0 g.) in tert-butyl alcohol (100 g.). Keep the reaction mixture at 30°-35° C. during the addition and stir for a further 3 hours. Neutralize the solution with dilute hydrochloric acid (1:4), dilute with water and extract with ether. Dry the ethereal extract with anhydrous magnesium sulfate and distil off the ether: the residue (ethyl n-propyl-2-cyanoethylmalonate; 11 g) solidifies on cooling in ice, and melts at 31°-32° after recrystallization from ice-cold ethyl alcohol.

### Preparation of Cyanoethylated Compound

A cyanoethylated diaminocyclohexane is prepared according to U.S. Pat. No. 6,245,932, which is incorporated herein by reference, with cyanoethylated methylcyclohexylamines are readily prepared in the presence of water.

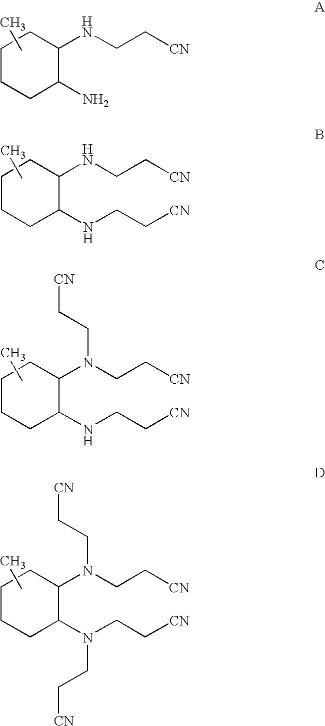

Analysis shows that almost no compounds exhibiting secondary amine hydrogen reaction and represented by structures C and D are produced when water alone is used as the catalytic promoter.

Examples of Reaction of Nitrile Compound with Hydroxylamine to Form Amidoxime Compound

Preparation and Analysis of Polyamidoxime (See, U.S. Pat. No. 3,345,344)

80 parts by weight of polyacrylonitrile of molecular weight of about 130,000 in the form of very fine powder (−300 mesh) was suspended in a solution of 300 parts by weight of hydroxylammonium sulfate, 140 parts by weight of sodium hydroxide and 2500 parts by weight of deionized water. The pH of the solution was 7.6. The mixture was heated to 90° C. and held at that temperature for 12 hours, all of the time under vigorous agitation. It was cooled to 35° C. and the product filtered off and washed repeatedly with deionized water. The resin remained insoluble throughout the reaction, but was softened somewhat by the chemical and heat. This caused it to grow from a very fine powder to small clusters of 10 to 20 mesh. The product weighed 130 grams. The yield 40 is always considerably more than theoretical because of firmly occluded salt. The product is essentially a polyamidoxime having the following reoccurring unit.

The mixture of hydroxylamine sulfate and sodium hydroxide can be replaced with equal molar of hydroxylamine freebase solution.

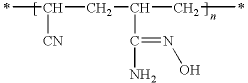

Portions of this product were then analyzed for total nitrogen and for oxime nitrogen by the well-known Dumas and Raschig methods and the following was found:

| | Percent |
|---|---|
| Total nitrogen (Dumas method) | 22.1 |
| Oxime nitrogen (Raschig method) | 6.95 |
| Amidoxime nitrogen (twice the amount of oxime | 13.9 |
| nitrogen) (calculated) | |
| Nitrile nitrogen (difference between the total | 8.2 |
| nitrogen and amidoxime nitrogen) (calculated) | |

Conversion of reacted product from cyanoethylation of cycloaliphatic vicinal primary amines (See, U.S. Pat. No. 6,245,932),

For example, Cyanoethylated methylcyclohexylamines

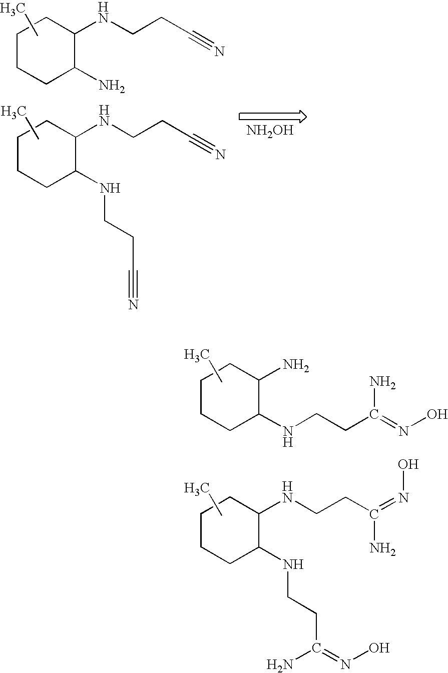

A large number of the amidoxime compounds are not commercially available. The amidoxime chelating compound can also be prepared in-situ while blending the cleaning formulation.

The following are photoresist stripper formulations that can be used with the amidoximes compounds of the present invention:

| | | | | Start | | After Step 1 | | After Step 2 | | End | | Stripper |
|---|---|---|---|---|---|---|---|---|---|---|---|---|
| | | Ingredient | MW | mole | Wt | mole | Wt | mole | Wt | mole | Wt | Composition |
| Step 1 | Amine | 2-Pyrolidone | 85.11 | 1.00 | 85.11 | 0.00 | 0.00 | 0.00 | 0.00 | 0.00 | 0.00 | 0% |
| | Nitrile | Acrylonitrile | 53.00 | 1.00 | 53.00 | 0.00 | 0.00 | 0.00 | 0.00 | 0.00 | 0.00 | 0% |
| | Metal Ion free | TMAH | 91.00 | 0.05 | 4.55 | 0.05 | 4.55 | 0.05 | 4.55 | 0.05 | 4.55 | 2% |
| | base | | | | | | | | | | | |
| | | Water | 18.00 | 0.76 | 13.65 | 0.76 | 13.65 | 0.76 | 13.70 | 0.76 | 13.68 | 6% |
| | Cyanoethylated | | 137.10 | 0.00 | 0.00 | 1.00 | 137.10 | 0.00 | 0.00 | 0.00 | 0.00 | 0% |
| | Compound | | | | | | | | | | | |
| Step 2 | Oxidizing/ | Hydroxylamine | 31.00 | 1.00 | 31.00 | 0.00 | 0.00 | 0.00 | 0.00 | 0.00 | 0.00 | 0% |
| | Reducing | | | | | | | | | | | |
| | compound | | | | | | | | | | | |
| | Water | Water | 18.00 | 1.72 | 31.00 | 0.00 | 0.00 | 1.72 | 31.00 | 1.72 | 31.00 | 14% |
| | Amidoxime | Amidoxime | 170.00 | 0.00 | 0.00 | 0.00 | 0.00 | 1.00 | 170.00 | 1.00 | 170.00 | 78% |

| | | |
|---|---|---|
| 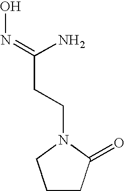 | 219.20 | 100% |

### Stripping Composition

| | Ingredient | Stripper Composition |
|---|---|---|
| Metal Ion free base | TMAH | 2% |
| Water | Water | 20% |
| Amidoxime | 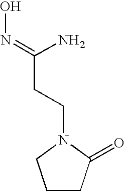 | 78% |
| | | 100% |

### Example of Amidoxime Derived from Ammonia

| 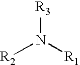 | | | 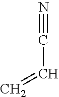 | H₂N—OH |
|---|---|---|---|---|
| R₁ | R₂ | R₃ | Nitrile | Amidoxime |
| —H | —H | —H | 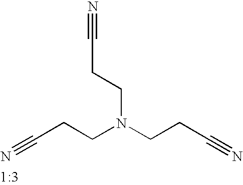 | 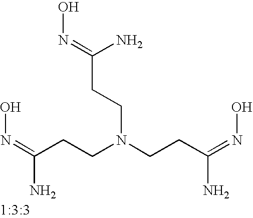 |
| CH₃CH₂ | H | H | 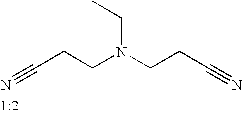 | 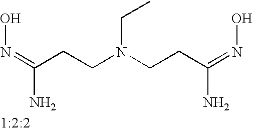 |
| CH₃CH₂ | CH₃CH₂ | H | 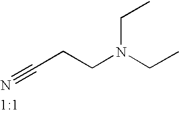 | 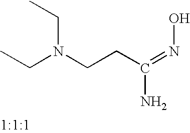 |

### Amidoxime Derived from Citric Acid

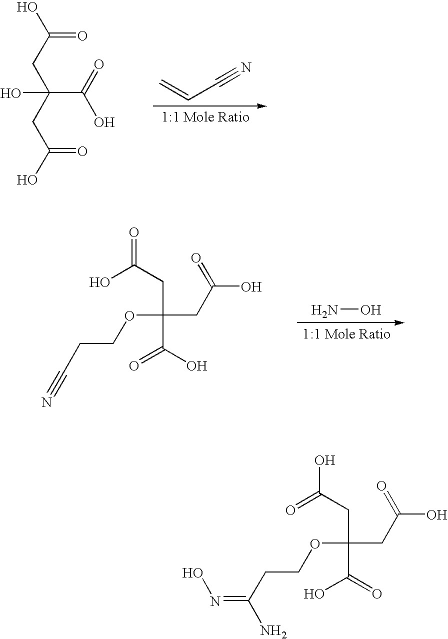

| Reactants | 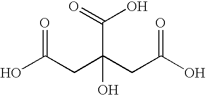 |
|---|---|
| CA:AN:HA 1:1:1 | 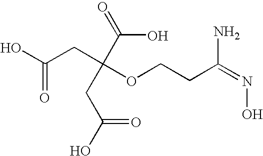 |
| CA:AN:HA 1:1:1 | 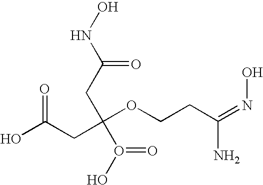 |
| CA:AN:HA 1:1:1 | 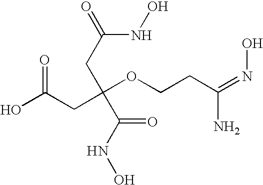 |
| CA:AN:HA 1:1:1 | 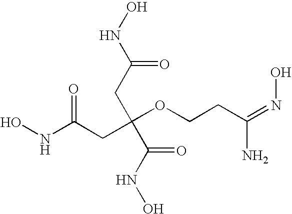 |

### Amidoxime Derived from Lactic Acid

| 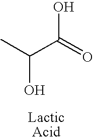 | 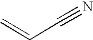 | Amidoxime Compounds | |
|---|---|---|---|
| — | 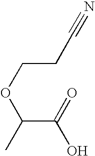 | 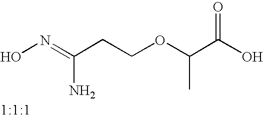 | 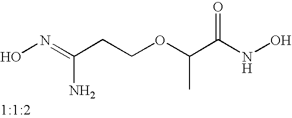 |

### Amidoxime Derived from Propylene Glycol

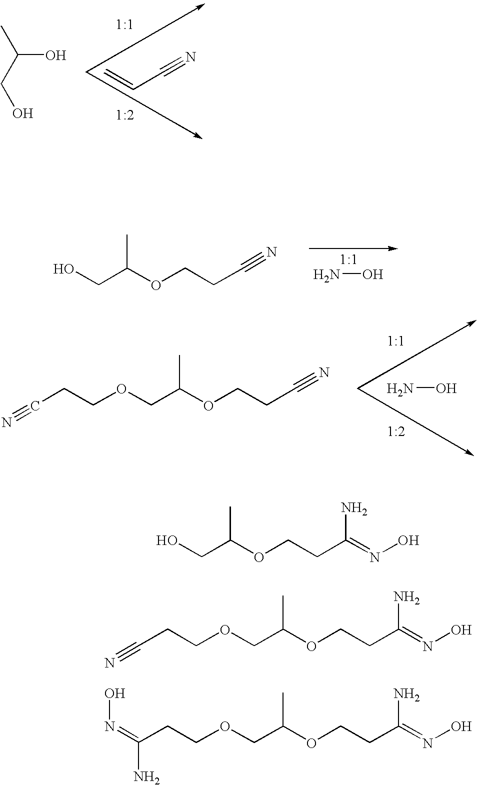

| | Amidoxime Compounds | | |
|---|---|---|---|
| Reactant | PG:AN:HA 1:1:1 | PG:AN:HA 1:2:1 | PG:AN:HA 1:2:2 |
| 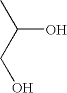 | 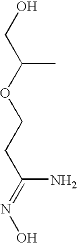 | 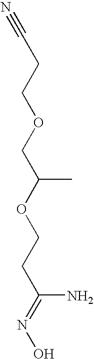 | 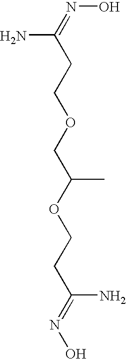 |

### Amidoxime Derived from Pentaerythritol—DS1

| | | | |
|---|---|---|---|
| 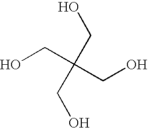 | 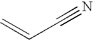 | H₂N—OH | Amidoxime Compounds |

| | | |
|---|---|---|
| 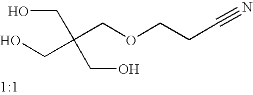 | 1 | 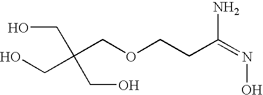 |

### Amidoxime Derived from Pentaerythritol—DS2

| | | | |
|---|---|---|---|
| 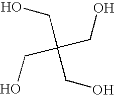 | 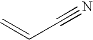 | H₂N—OH | Amidoxime Compounds |

| | | |
|---|---|---|
| 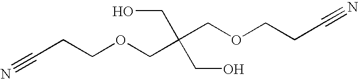 | 1 | 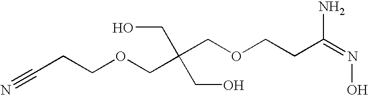 |
| 1:2 | 2 | 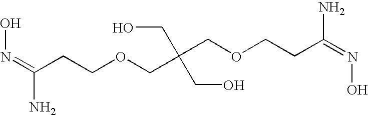 |

### Amidoxime Derived from Pentaerythritol—DS3

| | | | |
|---|---|---|---|
| 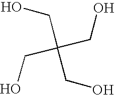 | 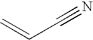 | H₂N—OH | Amidoxime Compounds |

| | | |
|---|---|---|
| 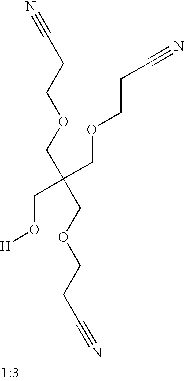 | 1 2 3 | 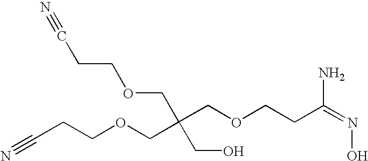 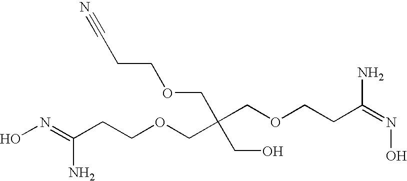 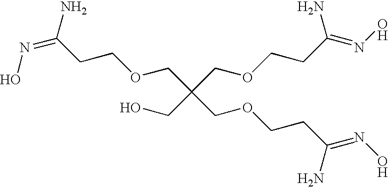 |

### Amidoxime Derived from Pentaerythritol—DS4

| | | | |
|---|---|---|---|
| 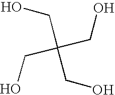 | 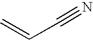 | H₂N—OH | Amidoxime Compounds |

| | | |
|---|---|---|
| 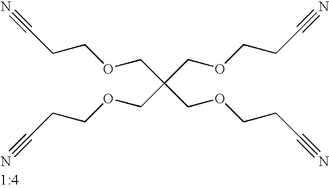 | 1 | 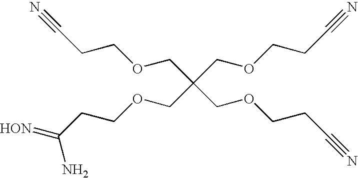 |
| | 2 | 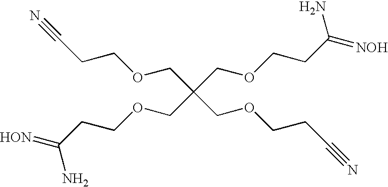 |
| | 3 | 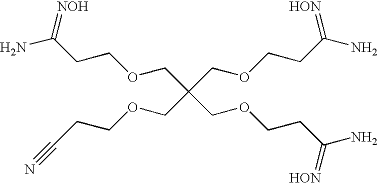 |
| | 4 | 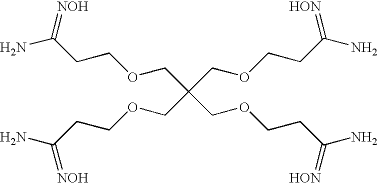 |

### α-Substituted Acetic Acid

| R | 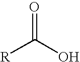 |
|---|---|
| —CH₃ | Acetic Acid |
| —CH₂OH | Glycolic Acid |
| —CH₂NH₂ | Glycine |
| —CHO | Glyoxylic Acid |

| | | H₂N—OH | | |
|---|---|---|---|---|
| 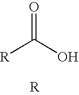 | 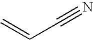 | 1 | 2 | 3 |
| —CH₃ | | 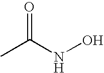 | | |
| —CH₂OH | 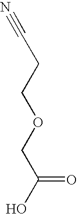 | 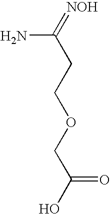 | 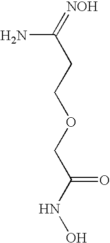 | |
| —CH₂NH₂ | 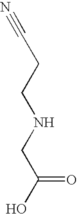 | 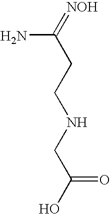 | 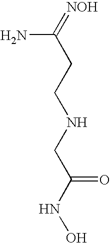 | |
| —CH₂NH₂ | 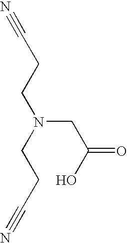 | 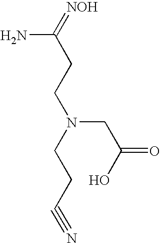 | 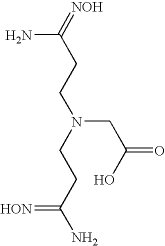 | 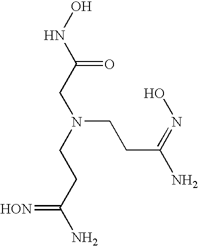 |
| —CHO | 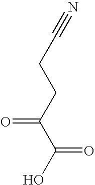 | 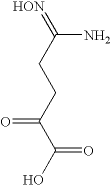 | 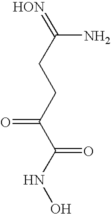 | |

### Amidoxime Derived from Iminodiacetic Acid

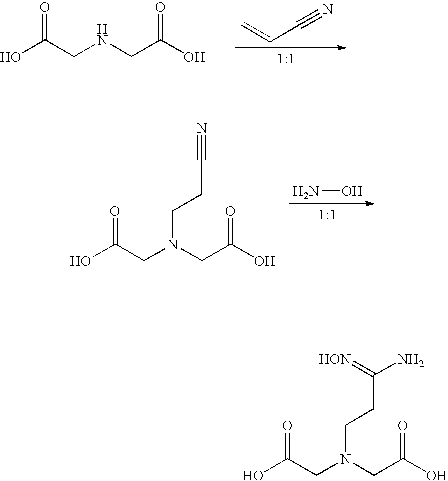

| Reactants | 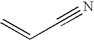 | H₂N—OH | 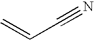 | H₂N—OH | 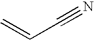 | H₂N—OH |
|---|---|---|---|---|---|---|
| 1 | 1 | 1 | 1 | 2 | 1 | 3 |

| | | | |
|---|---|---|---|
| 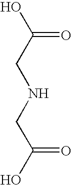 | 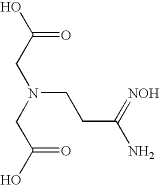 | 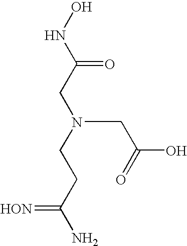 | 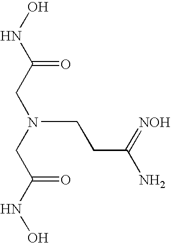 |

### Amidoxime Derived from 2,5-piperazinedione

| Reactants | 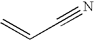 | H₂N—OH | 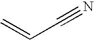 | H₂N—OH | 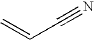 | H₂N—OH |
|---|---|---|---|---|---|---|
| 1 | 1 | 1 | 2 | 1 | 2 | 2 |

| | | | |
|---|---|---|---|
| 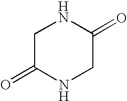 | 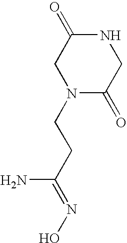 | 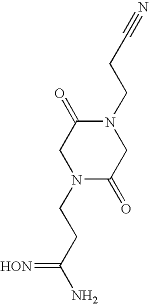 | 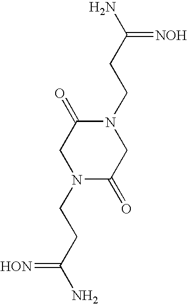 |

### Amidoxime Derived from Cyanopyridine

| Reactants | H₂N—OH | 1594-57-6 |
|---|---|---|
| 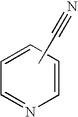 | 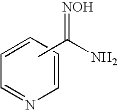 | 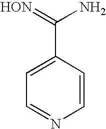 |
| 2, 3 or 4 Cyanopyridine | 2, 3 or 4 Amidoxime pyridine | 4-Amidoxime-pyridine |

Nomenclatures are translated from chemical structures to their corresponding chemical names using ChemBioDraw Ultra from CambridgeSoft, MA. In the case for products from the reaction of sorbitol, the cyanoethylated sorbitol is given by its CAS#[2465-92-1] as 1,2,3,4,5,6-hexakis-O-(2-cyanoethyl)hexitol with chemical formula of C₂₄H₃₂N₆O₆, and the corresponding amidoxime compound as 1,2,3,4,5,6-hexakis-O-[3-(hydroxyamino)-3-iminopropyl Hexitol, CAS#[950752-25-7].

Reactions to Produce Nitrile Precursors to Amidoxime Compounds

### Cyanoethylation of Diethylaminexine

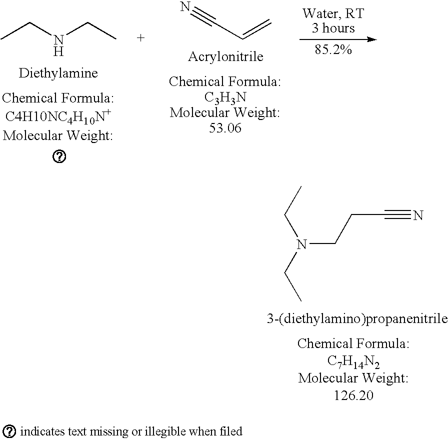

A solution of diethylamine (1 g, 13.67 mmol) and acrylonitrile (0.798 g, 15 mmol, 1.1 eq) in water (10 cm³) were stirred at room temperature for 3 hours, after which the mixture was extracted with dichloromethane (2×50 cm³). The organic extracts were evaporated under reduced pressure to give the pure cyanoethylated compound 3-(diethylamino)propanenitrile (1.47 g, 85.2%) as an oil.

### Monocyanoethylation of Glycine

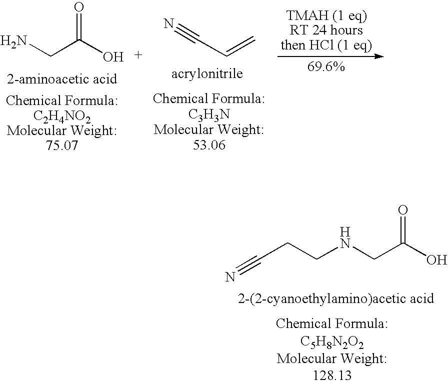

Glycine (5 g, 67 mmol) was suspended in water (10 cm³) and TMAH (25% in water, 24.3 g, 67 mmol) was added slowly, keeping the temperature at <30° C. with an ice-bath. The mixture was then cooled to 10° C. and acrylonitrile (3.89 g, 73 mmol) was added. The mixture was stirred overnight, and allowed to warm to room temperature slowly. The mixture was then neutralized with HCl (6M, 11.1 cm³), concentrated to 15 cm³ and diluted to 100 cm³ with EtOH. The solid precipitated was collected by filtration, dissolved in hot water (6 cm³) and reprecipitated with EtOH (13 cm³) to give 2-(2-cyanoethylamino)acetic acid (5.94 g, 69.6%) as a white solid, mp 192° C. (lit mp 190-191° C.).

### Cyanoethylation of Piperazinexine

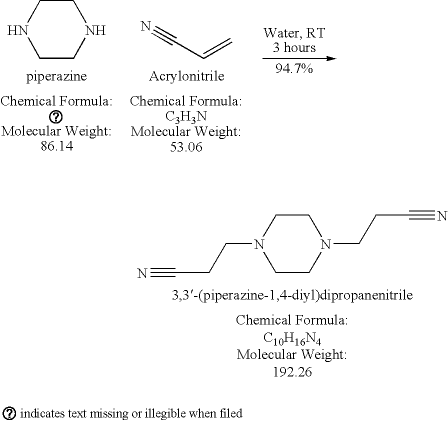

A solution of piperazine (1 g, 11.6 mmol) and acrylonitrile (1.6 g, 30.16 mmol, 2.6 eq) in water (10 cm3) were stirred at room temperature for 5 hours, after which the mixture was extracted with dichloromethane (2×50 cm3). The organic extracts were evaporated under reduced pressure to give the pure doubly cyanoethylated compound 3,3′-(piperazine-1,4-diyl)dipropanenitrile (2.14 g, 94.7%) as a white solid, mp 66-67° C.

### Cyanoethylation of 2-ethoxyethanol

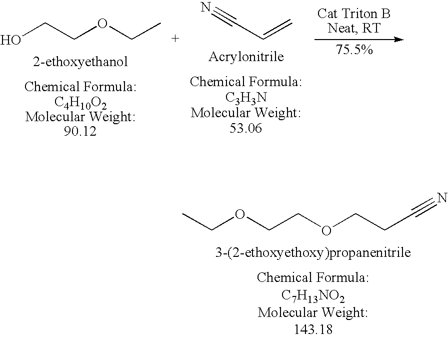

To an ice-water cooled mixture of 2-ethoxyethanol (1 g, 11.1 mol) and Triton B (40% in MeOH, 0.138 g, 0.33 mmol) was added acrylonitrile (0.618 g, 11.6 mmol) and the mixture was stirred at room temperature for 24 hours. It was then neutralized with 0.1 M HCl (3.3 cm3) and extracted with CH2Cl2 (2×10 cm3) The extracts were concentrated under reduced pressure and the residue was Kugelrohr-distilled to give the product 3-(2-ethoxyethoxy)propanenitrile (1.20 g, 75.5%) as a colourless oil, bp 100-130° C./20 Torr.

### Cyanoethylation of 2-(2-dimethylaminoethoxy)ethanol

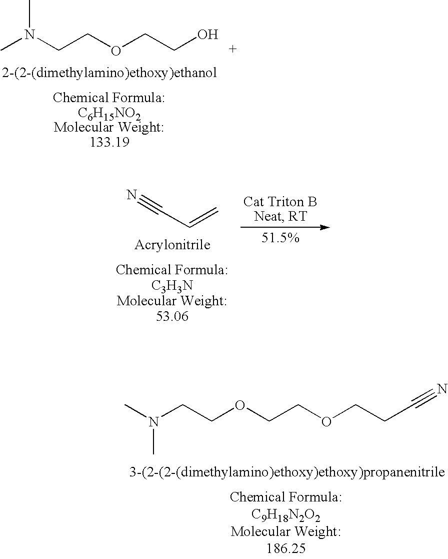

To an ice-water cooled mixture of 2-(2-dimethyleminothoxy)ethanol (1 g, 7.5 mmol) and Triton B (40% in MeOH, 0.094 g, 0.225 mmol) was added acrylonitrile (0.418 g, 7.9 mmol) and the mixture was stirred at room temperature for 24 hours. It was then neutralized with 0.1 M HCl (2.3 cm³) and extracted with CH₂Cl₂ (2×10 cm³). The extracts were concentrated under reduced pressure and the residue was purified by column chromatography (silica, Et₂O, 10% CH₂C₂, 0-10% EtOH) to give 3-(2-(2-(dimethylamino)ethoxy)ethoxy)propanenitrile as an oil.

### Cyanoethylation of Isobutyraldehyde

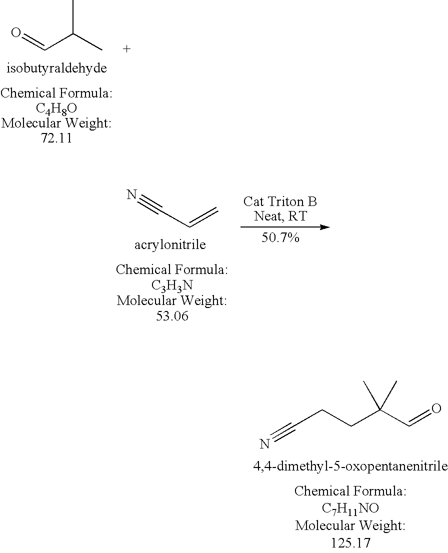

Isobutyraldehyde (1 g, 13.9 mmol) and acrylonitrile (0.81 g, 15 mmol) were mixed thoroughly and cooled with an ice-bath. Triton B (40% in MeOH, 0.58 g, 1.4 mmol) was added. The mixture was stirred at room temperature overnight. It was then neutralized with 0.1 M HCl (14 cm³) and extracted with CH₂Cl₂ (100 cm³) The extracts were concentrated under reduced pressure and the residue was Kugelrohr-distilled to give the product 4,4-dimethyl-5-oxopentanenitrile (0.8 g, 50.7%) as an oil, bp 125-130° C./20 Torr.

### Cyanoethylation of Aniline

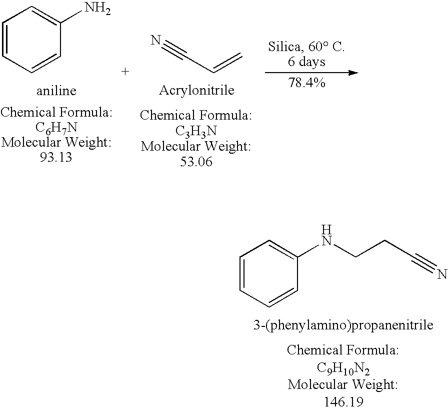

Silica was activated by heating it above 100° C. in vacuum and was then allowed to cool to room temperature under nitrogen. To the activated silica (10 g) was absorbed aniline (1.86 g, 20 mmol) and acrylonitrile (2.65 g, 50 mmol) and the flask was capped tightly. The contents were then stirred with a magnetic stirrer for 6 days at 60° C. After this time the mixture was cooled to room temperature and extracted with MeOH. The extracts were evaporated to dryness and the residue was Kugelrohr-distilled under high vacuum to give the product 3-(phenylamino)propanenitrile (2.29 g, 78.4%) as an oil which crystallised on standing; bp 120-150° C./1-2 Torr (lit bp 120° C./1 Torr), mp 50.5-52.5° C.

### Cyanoethylation of Ethylenediamine

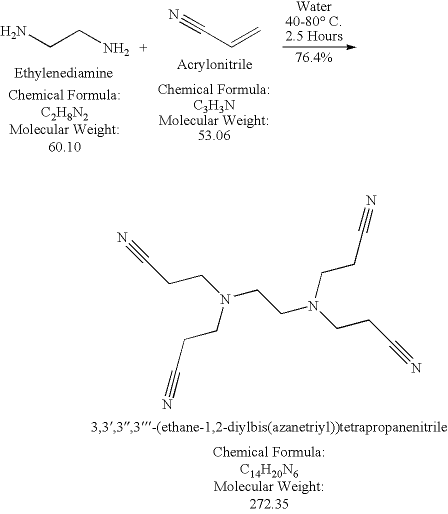

Acrylonitrile (110 g, 137 cm3, 2.08 mol) was added to a vigorously stirred mixture of ethylenediamine (25 g, 27.8 cm3, 0.416 mol) and water (294 cm3) at 40° C. over 30 min. During the addition, it was necessary to cool the mixture with a 25° C. water bath to maintain temperature at 40° C. The mixture was then stirred for additional 2 hours at 40° C. and 2 hours at 80° C. Excess acrylonitrile and half of the water were evaporated off and the residue, on cooling to room temperature, gave a white solid which was recrystallised from MeOH-water (9:1) to give pure product 3,3′,3″,3′″-(ethane-1,2-diylbis(azanetriyl))tetrapropanenitrile (86.6 g, 76.4%) as white crystals, mp 63-65° C.

### Cyanoethylation of Ethylene Glycol

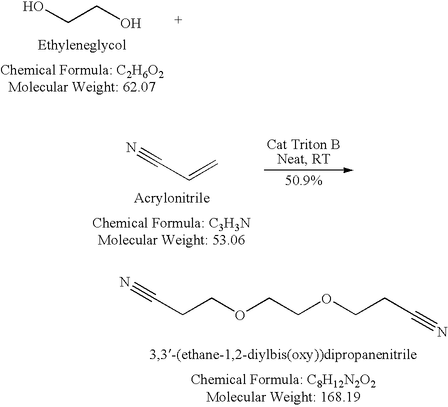

Small scale: Ethylene glycol (1 g, 16.1 mmol) was mixed with Triton B (40% in MeOH, 0.22 g, 0.53 mmol) and cooled in an ice-bath while acrylonitrile (1.71 g, 32.2 mmol) was added. The mixture was stirred at room temperature for 60 hours after which it was neutralized with 0.1 M HCl (0.6 cm³) and extracted with CH₂Cl₂ (80 cm³) The extracts were concentrated under reduced pressure and the residue was Kugelrohr-distilled to give 3,3′-(ethane-1,2-diylbis(oxy))dipropanenitrile (1.08 g, 39.9%) as a light coloured oil, hp 150-170° C./20 Torr.

Large scale: Ethylene glycol (32.9 g, 0.53 mol) was mixed with Triton B (40% in MeOH, 2.22 g, 5.3 mmol) and cooled in an ice-bath while acrylonitrile (76.2 g, 1.44 mol) was added. The mixture was allowed to warm slowly to room temperature and stirred for 60 hours after which it was neutralized with 0.1 M HCl (50 cm³) and extracted with CH₂Cl₂ (300 cm³) The extracts were passed through a silica plug three times to reduce the brown coloring to give 86 g (quantitative yield) of the product as an amber coloured oil, pure by ¹H-NMR, containing 10 g of water (total weight 96 g, amount of water calculated by ¹H NMR integral sizes).

### Cyanoethylation of Diethyl Malonate

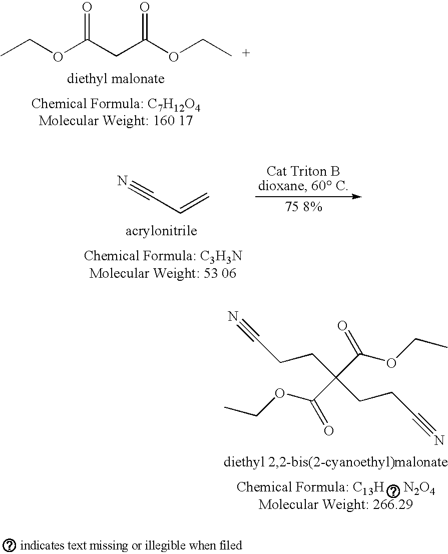

To a solution of diethyl malonate (1 g, 6.2 mmol) and Triton B (40% in MeOH, 0.13 g, 0.31 mmol) in dioxane (1.2 cm³) was added dropwise acrylonitrile (0.658 g, 12.4 mmol) and the mixture was stirred at 60° C. overnight. The mixture was then cooled to room temperature and neutralized with 0.1 M HCl (3 cm³) and poured to ice-water (10 cm³). Crystals precipitated during 30 min. These were collected by filtration and recrystallised from EtOH (cooling in freezer before filtering off) to give diethyl 2,2-bis(2-cyanoethyl)malonate (1.25 g, 75.8%) as a white solid, mp 62.2-63.5° C.

### Hydrolysis of Diethyl 2,2-bis(2-cyanoethyl)malonate

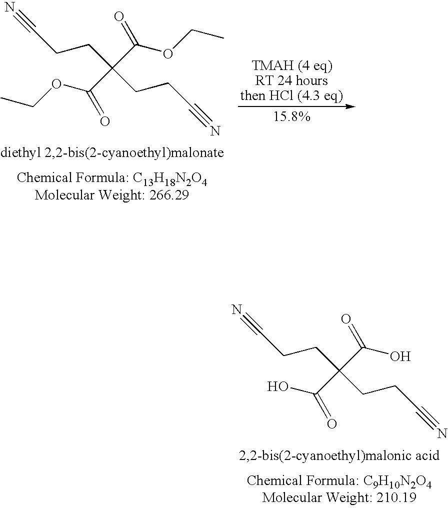

Diethyl 2,2-bis(2-cyanoethyl)malonate (2 g, 7.51 mmol) was added to TMAH (25% in water, 10.95 g, 30.04 mmol) at room temperature. The mixture was stirred for 24 hours, and was then cooled to 0° C. A mixture of 12M HCl (2.69 cm³, 32.1 mmol) and ice (3 g) was added and the mixture was extracted with CH₂Cl₂ (5×50 cm³). The extracts were evaporated under vacuum to give 2,2-bis(2-cyanoethyl)malonic acid (0.25 g, 15.8%) as a colourless very viscous oil (lit decomposed. 158° C.).

### Dicyanoethylation of Glycine to Give 2-(bis(2-cyanoethyl)amino)acetic Acid

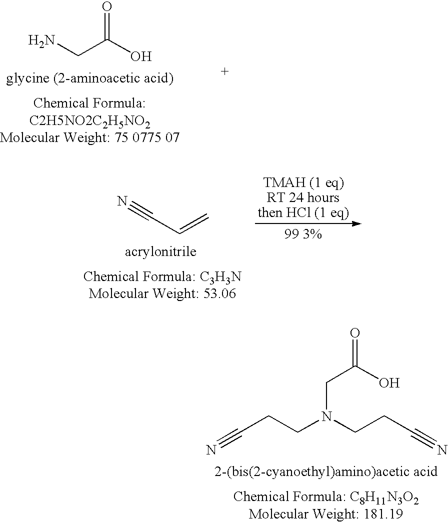

Glycine (5 g, 67 mmol) was suspended in water (10 cm³) and TMAH (25% in water, 24-3 g, 67 mmol) was added slowly, keeping the temperature at <30° C. with an ice-bath. The mixture was then cooled to 10° C. and acrylonitrile (7.78 g, 146 mmol) was added. The mixture was stirred overnight, and allowed to warm to room temperature slowly. It was then heated at 50° C. for 2 hours, using a reflux condenser. After cooling with ice, the mixture was neutralized with HCl (6M, 11.1 cm³) and concentrated to a viscous oil. This was dissolved in acetone (100 cm³) and filtered to remove NMe₄Cl. The filtrate was concentrated under reduced pressure to give an oil that was treated once more with acetone (100 cm³) and filtered to remove more NMe₄Cl. Concentration of the filtrate gave 2-(bis(2-cyanoethyl)amino)acetic acid (11.99 g, 99.3%) as a colourless, viscous oil that crystallised over 1 week at room temperature to give a solid product, mp 73° C. (lit mp 77.8-78.8° C. Duplicate ¹³C signals indicate a partly zwitterionic form in CDCl₃ solution.

When NaOH is used in the literature procedure, the NaCl formed is easier to remove and only one acetone treatment is necessary.

### Dicyanoethylation of N-methyldiethanolamine to Give 3,3′-(2,2′-(methylazanediyl)bis(ethane-2,1-diyl)bis(oxy))dipropanenitrile

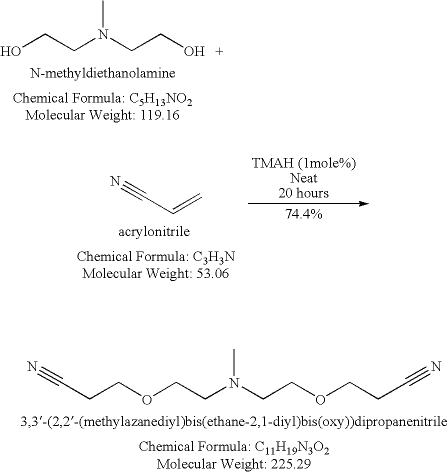

To a cooled, stirred mixture of N-methyldiethanolamine (2 g, 17 mmol) and acrylonitrile (2.33 g, 42 mmol) was added TMAH (25% in water, 0.25 cm³, 0.254 g, 7 mmol). The mixture was then stirred overnight, and allowed to warm to room temperature slowly. It was then filtered through silica using a mixture of Et₂O and CH₂Cl₂ (1:1, 250 cm³) and the filtrated was evaporated under reduced pressure to give 3,3′-(2,2′-(methylazanediyl)bis(ethane-2,1-diyl)bis(oxy))dipropanenitrile (2.85 g, 74.4%) as a colourless oil.

### Dicyanoethylation of Glycine Anhydride

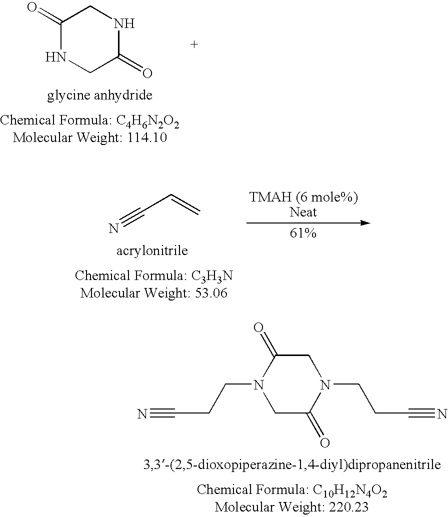

Glycine anhydride (2 g, 17.5 mmol) was mixed with acrylonitrile (2.015 g, 38 mmol) at 0° C. and TMAH (25% in water, 0.1 cm³, 0.1 g, 2.7 mmol) was added. The mixture was then stirred overnight, allowing it to warm to room temperature slowly. The solid formed was recrystallised from EtOH to give 3,3′-(2,5-dioxopiperazine-1,4-diyl)dipropanenitrile (2.35 g, 61%) as a white solid, mp 171-173° C. (lit mp 166° C.).

### N,N-Dicyanoethylation of Acetamide

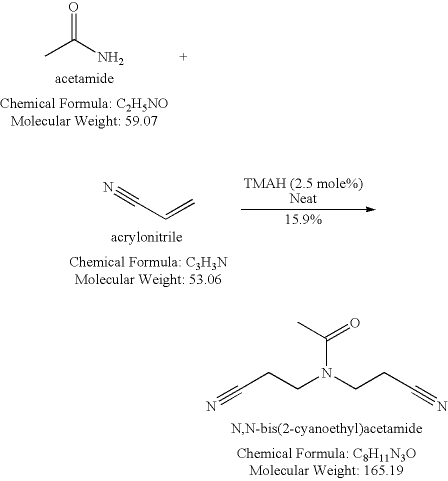

Acetamide (2 g, 33.9 mmol) was mixed with acrylonitrile (2.26 g, 42.7 mmol) at 0° C. and TMAH (25% in water, 0.06 cm³, 0.06 g, 1.7 mmol) was added. The mixture was then stirred overnight, allowing it to warm to room temperature slowly. The mixture was filtered through a pad of silica with the aid of Et₂O/CH₂Cl₂ (200 cm³) and the filtrate was concentrated under reduced pressure. The product was heated with spinning in a Kugelrohr at 150° C./2 mmHg to remove side products and to give N,N-bis(2-cyanoethyl)acetamide (0.89 g, 15.9%) as a viscous oil.

The N-Substituent in the Amides is Non-Equivalent Due to Amide Rotation.

### Tricyanoethylation of Ammonia

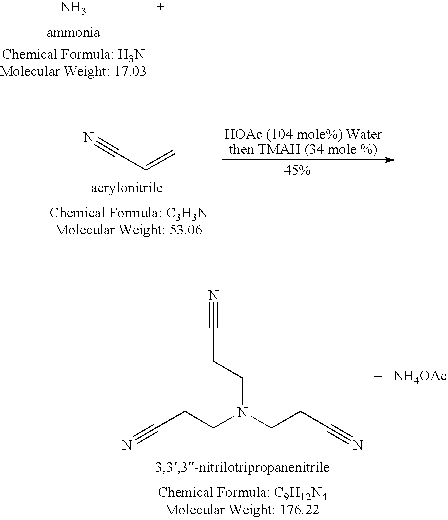

Ammonia (aq 35%, 4.29, 88 mmol) was added dropwise to ice-cooled AcOH (5.5 g, 91.6 mmol) in water (9.75 cm³), followed by acrylonitrile (4.65 g, 87.6 mol). The mixture was stirred under reflux for 3 days, after which it was cooled with ice and aq TMAH (25% in water, 10.94 g, 30 mmol) was added. The mixture was kept cooled with ice for 1 hours. The crystals formed was collected by filtration and washed with water. The product was dried in high vacuum to give 3,3′,3″-nitrilotripropanenitrile (2.36 g, 45.8%) as a white solid, mp 59-61° C. (lit mp 59° C.).

When NaOH was used to neutralize the reaction (literature procedure), the yield was higher, 54.4%.

### Dicyanoethylation of Cyanoacetamide

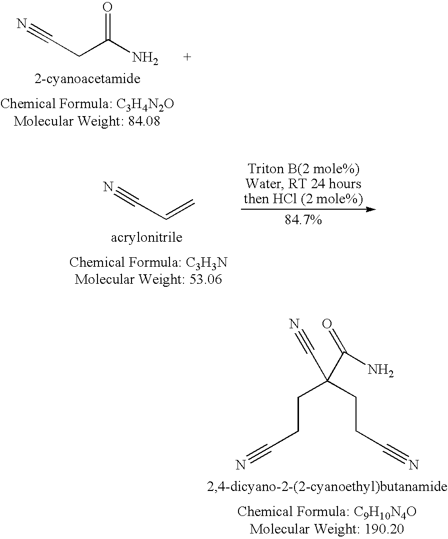

To a stirred mixture of cyanoacetamide (2.52 g, 29.7 mmol) and Triton B (40% in MeOH, 0.3 g, 0.7 mmol) in water (5 cm³) was added acrylonitrile (3.18 g, 59.9 mmol) over 30 minutes with cooling. The mixture was then stirred at room temperature for 30 min and then allowed to stand for 1 hours. EtOH (20 g) and 1M HCl (0.7 cm³) were added and the mixture was heated until all solid had dissolved. Cooling to room temperature gave crystals that were collected by filtration and recrystallised from EtOH to give 2,4-dicyano-2-(2-cyanoethyl)butanamide (4.8 g, 84.7%) as a pale yellow solid, mp 118-120° C. (lit. mp 118° C.),

### N,N-Dicyanoethylation of Anthranilonitrile

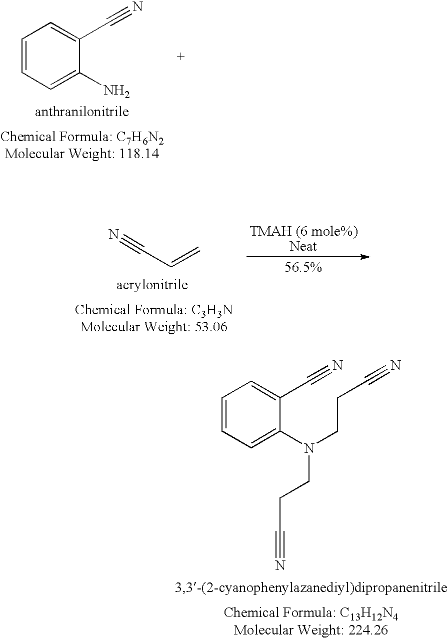

Anthranilonitrile (2 g, 16.9 mmol) was mixed with acrylonitrile (2.015 g, 38 mmol) at 0° C. and TMAH (25% in water, 0.1 cm³, 0.1 g, 2.7 mmol) was added. The mixture was then stirred overnights allowing it to warm to room temperature slowly. The product was dissolved in CH₂Cl₂ and filtered through silica using a mixture of Et₂O and CH₂Cl₂ (1:1, 250 cm³). The filtrate was evaporated to dryness and the solid product was recrystallised from EtOH (5 cm³) to give 3,3′-(2-cyanophenylazanediyl)dipropanenitrile (2.14 g, 56.5%) as an off-white solid, mp 79-82° C.

### Dicyanoethylation of Malononitrile

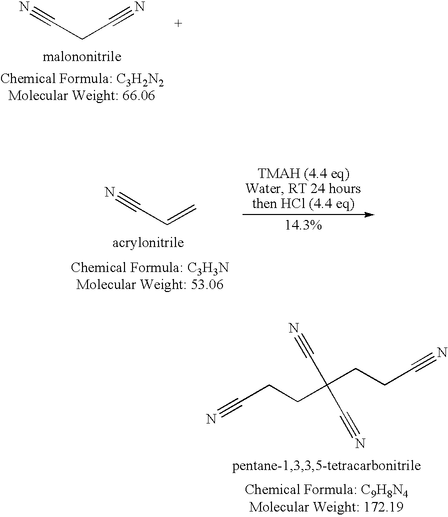

Malononitrile (5 g, 75.7 mmol) was dissolved in dioxane (10 cm³), followed by trimethylbenzylammonium hydroxide (Triton B, 40% in MeOH, 1.38 g, 3.3 mmol). The mixture was cooled while acrylonitrile (8.3 g, 156 mmol) was added. The mixture was stirred overnight, allowing it to warm to room temperature slowly. It was then neutralized with HCl (1 M, 3.3 cm³) and poured into ice-water. The mixture was extracted with CH₂Cl₂ (200 cm³) and the extracts were evaporated under reduced pressure. The product was purified by column chromatography (silica, 1:1 EtOAc-petroleum) followed by recrystallisation to give 1,3,3,5-tetracarbonitrile (1.86 g, 14.3%), mp 90-92° C. (lit mp 92° C.).

### Tetracyanoethylation of Pentaerythritol

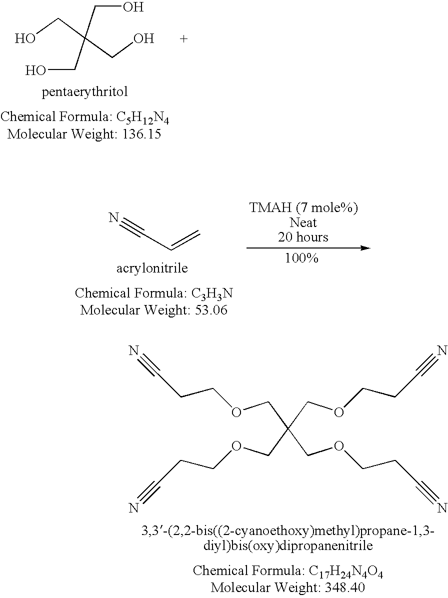

Pentaerythritol (2 g, 14.7 mmol) was mixed with acrylonitrile (5 cm³, 4.03 g, 76 mmol) and the mixture was cooled in an ice-bath while tetramethylammonium hydroxide (=TMAH, 25% in water, 0.25 cm³, 0.254 g, 7 mmol) was added. The mixture was then stirred at room temperature for 20 hours. After the reaction time the mixture was filtered through silica using a mixture of Et₂O and CH₂Cl₂ (1:1, 250 cm³) and the filtrated was evaporated under reduced pressure to give 3,3′-(2,2-bis((2-cyanoethoxy)methyl)propane-1,3-diyl)bis(oxy)dipropanenitrile (5.12 g, 100%) as a colourless oil.

### Hexacyanoethylation of Sorbitol

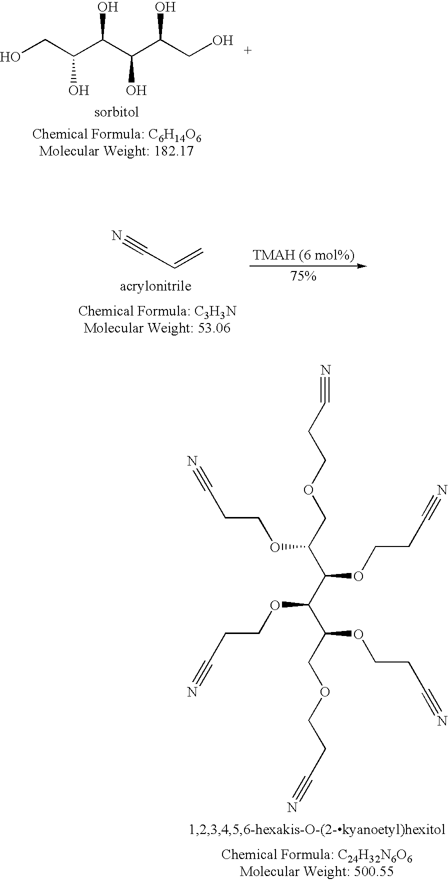

Sorbitol (2 g, 11 mmol) was mixed with acrylonitrile (7 cm³, 5.64 g, 106 mmol) and the mixture was cooled in an ice-bath while tetramethylammonium hydroxide (=TMAH, 25%; in water, 0.25 cm³, 0.254 g, 7 mmol) was added. The mixture was then stirred at room temperature for 48 hours, adding another 0.25 cm³ of TMAH after 24 hours. After the reaction time the mixture was filtered through silica using a mixture of Et₂O and CH₂Cl₂ (1:1, 250 cm³) and the filtrate was evaporated under reduced pressure to give a fully cyanoethylated product (4.12 g, 75%) as a colourless oil.

### Tricyanoethylation of Diethanolamine to Give 3,3′-(2,2′-(2-cyanoethylazanediyl)bis(ethane-2,1-diyl)bis(oxy))dipropanenitrile

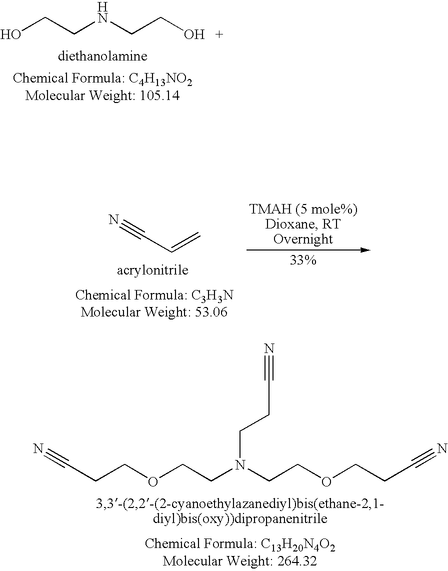

To an ice-cooled stirred solution of diethanolamine (2 g, 19 mmol) and TMAH (25% in water, 0.34 cm³, 0.35 g, 9.5 mmol) in dioxane (5 cm³) was added acrylonitrile (3.53 g, 66.1 mmol) dropwise. The mixture was then stirred overnight, and allowed to warm to room temperature. More acrylonitrile (1.51 g, 28 mmol) and TMAH (0.25 cm³, 7 mmol) was added and stirring was continued for additional 24 h. The crude mixture was filtered through a pad of silica (Et₂O/CH₂Cl₂ as eluent) and evaporated to remove dioxane. The residue was purified by column chromatography (silica, Et₂O to remove impurities followed by EtOAc to elute product) to give 3,3′-(2,2′-(2-cyanoethylazanediyl)bis(ethane 2,1-diyl)bis(oxy))dipropanenitrile (1.67 g, 33%) as an oil.

Reactions to Produce Amidoxime Compounds

### Reaction of Acetonitrile to Give N′-hydroxyacetimidamide

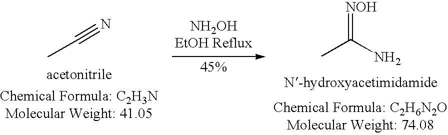

A solution of acetonitrile (0.78 g, 19 mmol) and hydroxylamine (50% in water, 4.65 cm³, 5.02 g, 76 mmol, 4 eq) in EtOH (100 cm³) was stirred under reflux for 1 hours, after which the solvent was removed under reduced pressure and the residue was recrystallised from iPrOH to give the product N′-hydroxyacetimidamide (0.63 g, 45%) as a solid, mp 134.5-136.5° C.

### Reaction of Octanonitrile to Give N′-hydroxyoctanimidamide

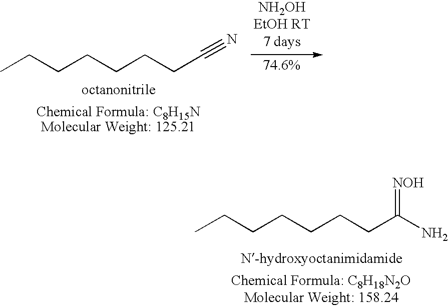

Octanonitrile (1 g, 7.99 mmol) and hydroxylamine (50% in water, 0.74 cm3, 0.79 g, 12 mmol, 1.5 eq) in EtOH (1 cm³) were stirred at room temperature for 7 days. Water (10 cm³) was then added. This caused crystals to precipitate, these were collected by filtration and dried in high vacuum line to give the product N′-hydroxyoctanimidamide (0.94 g, 74.6%) as a white solid, mp 73-75° C.

### Reaction of Chloroacetonitrile to Give 2-chloro-N′-hydroxyacetimidamide

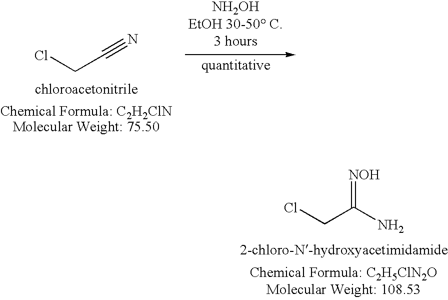

Chloroacetonitrile (1 g, 13 mmol) and hydroxylamine (50% in water, 0.89 cm³, 0.96 g, 14.6 mmol, 1.1 eq) in EtOH (1 cm³) were stirred at 30-50° C. for 30 min. The mixture was then extracted with Et2O (3×50 cm³). The extracts were evaporated under reduced pressure to give the product 2-chloro-N′-hydroxyacetimidamide (0.81 g, 57.4%) as a yellow solid, mp 79-80° C.

### Reaction of ethyl 2-cyanoacetate to Give 3-amino-N-hydroxy-3-(hydroxyimino)propanamide

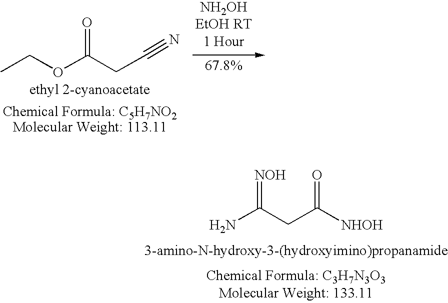

Ethyl cyanoacetate (1 g, 8.84 mmol) and hydroxylamine (50% in water, 1.19 cm3, 1.29 g, 19.4 mmol, 2.2 eq) in EtOH (1 cm³) were allowed to stand at room temperature for 1 hour with occasional swirling. The crystals formed were collected by filtration and dried in high vacuum line to give a colourless solid, 3-amino-N-hydroxy-3-(hydroxyimino)propanamide, mp 158° C. (decomposed) (lit mp 150° C.).

### Reaction of 3-hydroxypropionitrile to Give N′,3-dihydroxypropanimidamide

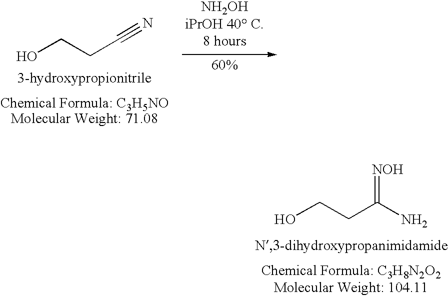

Equal molar mixture of 3-hydroxypropionitrile and hydroxylamine heated to 40° C. for 8 hours with stirring. The solution is allowed to stand overnight yielding a fine slightly off white precipitate. The precipitated solid was filtered off and washed with iPrOH and dried to a fine pure white crystalline solid N′,3-dihydroxypropanimidamide mp 94° C.

### Reaction of 2-cyanoacetic Acid to Give Isomers of 3-amino-3-(hydroxyimino)propanoic acid

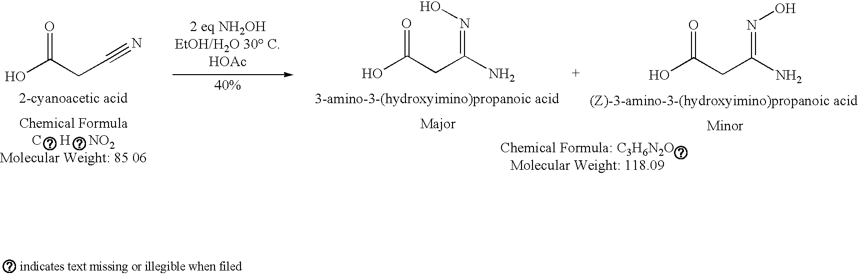

2-Cyanoacetic acid (1 g, 11.8 mmol) was dissolved in EtOH (10 cm³) and hydroxylamine (50% in water, 0.79 cm3, 0.85 g, 12.9 mmol, 1.1 eq) was added. The mixture was warmed at 40° C. for 30 min and the crystals formed (hydroxylammonium cyanoacetate) were filtered off and dissolved in water (5 cm³). Additional hydroxylamine (50% in water, 0.79 cm3, 0.85 g, 12.9 mmol, 1.1 eq) was added and the mixture was stirred at room temperature overnight. Acetic acid (3 cm³) was added and the mixture was allowed to stand for a few hours. The precipitated solid was filtered off and dried in high vacuum line to give the product 3-amino-3-(hydroxyimino)propanoic acid (0.56 g, 40%) as a white solid, mp 136.5° C. (lit 144° C.) as two isomers.

Characterization of the product using FTIR and NMR are as follows: νmax(KBr)/cm−1 3500−3000 (br), 3188, 2764, 1691, 1551, 1395, 1356, 1265 and 1076; δH (300 MHz; DMSO-d6; Me4Si) 10.0-9.0 (br, NOH and COOH), 5.47 (2H, br s, NH₂) and 2.93 (2H, s, CH₂); δC (75 MHz; DMSO-d6; Me4Si) 170.5 (COOH minor isomer), 170.2 (COOH major isomer), 152.8 (C(NOH)NH₂ major isomer) 148.0 (C(NOH)NH₂ minor isomer), 37.0 (CH₂ minor isomer) and 34.8 (CH₂ major isomer).

### Reaction of Adiponitrile to Give N′1,N′6-dihydroxyadipimidamide

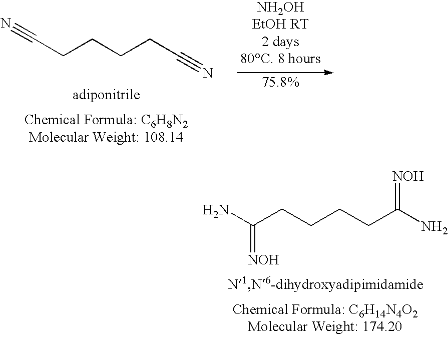

Adiponitrile (1 g, 9 mmol) and hydroxylamine (50% in water, 1.24 cm3, 1.34 g, 20 mmol, 2.2 eq) in EtOH (10 cm3) were stirred at room temperature for 2 days and then at 80° C. for 8 hours. The mixture was allowed to cool and the precipitated crystals were collected by filtration and dried in high vacuum line to give the product N′1,N′6-dihydroxyadipimidamide (1.19 g, 75.8%) as a white solid, mp 160.5 (decomposed) (lit decomposed 168-170° C.

### Reaction of Sebaconitrile to Give N′1,N′10-dihydroxydecanebis(imidamide)

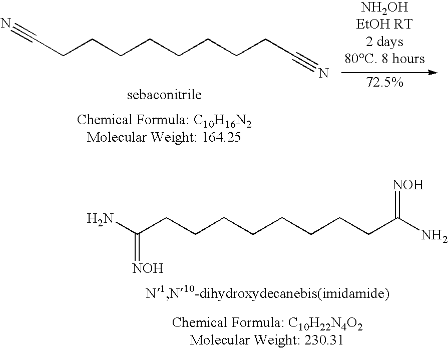

Sebaconitrile (1 g, 6 mmol) and hydroxylamine (50% in water, 0.85 cm³, 0.88 g, 13.4 mmol, 2.2 eq) in EtOH (12 cm³) were stirred at room temperature for 2 days and then at 80° C. for 8 h. The mixture was allowed to cool and the precipitated crystals were collected by filtration and dried in high vacuum line to give the product N′1,N′10-dihydroxydecanebis(imidamide) (1 g, 72.5%); mp 182° C.

### Reaction of 2-cyanoacetamide to Give 3-amino-3-(hydroxyimino)propanamide

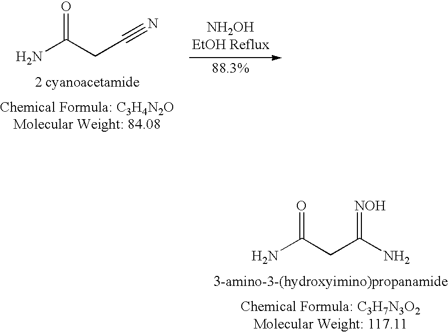

2-Cyanoacetamide (1 g, 11.9 mmol) and hydroxylamine (0.8 cm³, 13 mmol, 1.1 eq) in EtOH (6 cm³) were stirred under reflux for 2.5 hours. The solvents were removed under reduced pressure and the residue was washed with CH₂Cl₂ to give the product 3-amino-3-(hydroxyimino)propanamide (1.23 g, 88.3%) as a white solid, mp 159° C.

### Reaction of Glycolonitrile to Give N′,2-dihydroxyacetimidamide

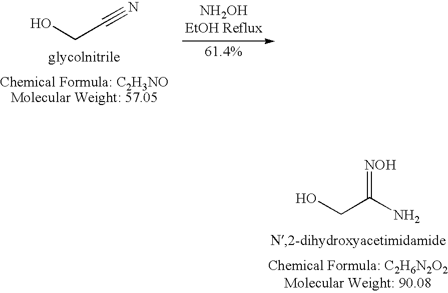

Glycolonitrile (1 g, 17.5 mmol) and hydroxylamine (50% in water, 2.15 cm³, 35 mmol, 2 eq) in EtOH (10 cm³) were stirred under reflux for 6 hours and then at room temperature for 2-4 hours. The solvent was evaporated and the residue was purified by column chromatography (silica, 1:3 EtOH-C₂Cl₂) to give the product N′,2-dihydroxyacetimidamide (0.967 g, 61.4%) as an off-white solid, mp 63-65° C.

### Reaction of 5-hexynenitrile to Give 4-cyano-N′-hydroxybutanimidamide

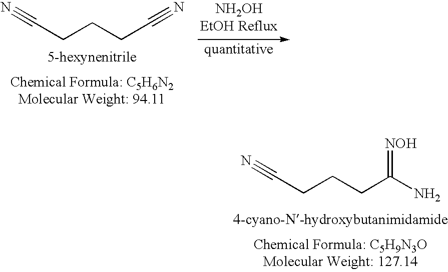

A solution of 5-hexynenitrile (0.93 g, 10 mmol) and hydroxylamine (50% in water, 1.22 cm³, 20 mmol) was stirred under reflux for 10 hours, after which volatiles were removed under reduced pressure to give the product 4-cyano-N′-hydroxybutanimidamide (1.30 g, 100%) as a white solid, mp 99.5-101° C.

### Reaction of Iminodiacetonitrile to Give 2,2′-azanediylbis(N′-hydroxyacetimidamide)

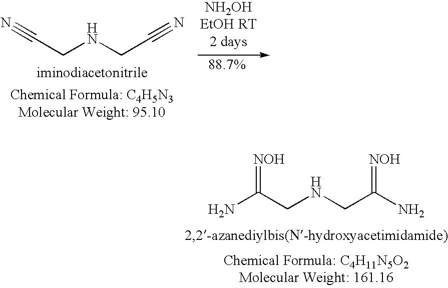

Commercial iminodiacetonitrile (Alfa-Aesar) was purified by dispersing the compound in water and extracting with dichloromethane, then evaporating the organic solvent from the extracts to give a white solid. Purified iminodiacetonitrile (0.82 g) and hydroxylamine (50% in water, 2.12 ml, 2.28 g, 34.5 mmol, 4 eq) in MeOH (6.9 ml) and water (6.8 ml) were stirred at room temperature for 48 hours. Evaporation of volatiles under reduced pressure gave a colorless liquid which was triturated with EtOH (40° C.) to give 2,2′-azanediylbis(N′-hydroxyacetimidamide) (1.23 g, 88.7%) as a white solid, mp 135-136° C., (lit mp 138° C.).

### Reaction of 3-methylaminopropionitrile to Give N′-hydroxy-3-(methylamino)propanimidamide

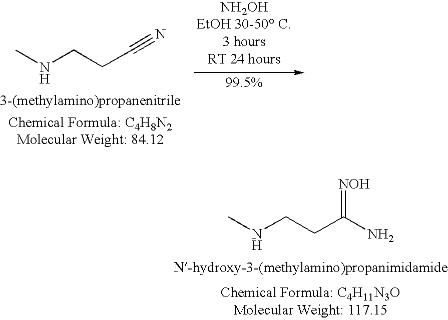

A solution of 3-methylaminopropionitrile (1 g, 11.9 mmol) and hydroxylamine (50% in water, 0.5 cm3, 0.864 g, 13.1 mmol, 1.1 eq) in EtOH (1 cm³) was stirred at 30-50° C. for 3 hours and then at room temperature overnight. The solvent was removed under reduced pressure (rotary evaporator followed by high vacuum line) to give the product N′-hydroxy-3-(methylamino)propanimidamide (1.387 g, 99.5%) as a thick pale yellow oil.

### Reaction of 3-(diethylamino)propanenitrile to Give 3-(diethylamino)-N′-hydroxypropanimidamide

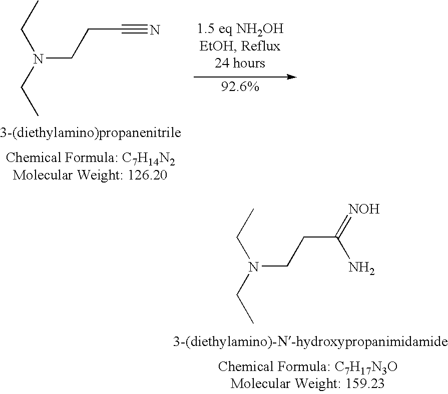

A solution of 3-(diethylamino)propanenitrile (1 g, 8 mmol) and NH₂OH (50% in water, 0.73 cm³, 11.9 mmol) in EtOH (10 cm³) were heated to reflux for 24 hours, after which the solvent and excess hydroxylamine were removed by rotary evaporator. The residue was freeze-dried and kept in high vacuum line until it slowly solidified to give 3-(diethylamino)-N′-hydroxypropanimidamide (1.18 g, 92.6%) as a white solid, mp 52-54° C.

### Reaction of 3,3′,3″-Nitrilotripropanenitrile with hydroxylamine to Give 3,3′,3″-nitrilotris(N′-hydroxypropanimidamide)

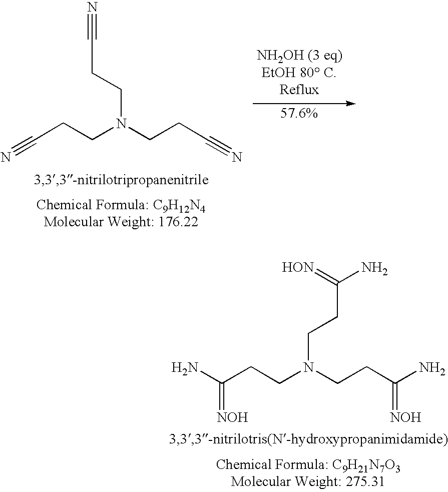

A solution of 3,3′,3″-nitrilotripropanenitrile (2 g, 11.35 mmol) and hydroxylamine (50% in water, 2.25 g, 34 mmol) in EtOH (25 cm³) was stirred at 80° C. overnight, then at room temperature for 24 hours. The white precipitate was collected by filtration and dried in high vacuum to give 3,3′,3″-nitrilotris(N′-hydroxypropanimidamide) (1.80 g, 57.6%) as a white crystalline solid, mp 195-197° C. (decomposed)

### Reaction of 3-(2-ethoxyethoxy)propanenitrile to Give 3-(2-ethoxyethoxy)-N′-hydroxypropanimidamide

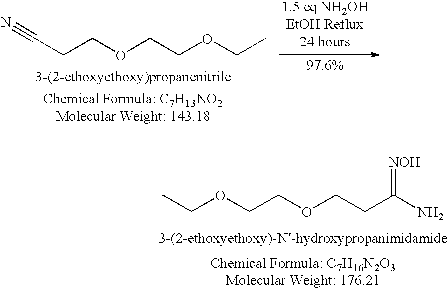

A solution of 3-(2-ethoxyethoxy)propanenitrile (1 g, 7 mmol) and NH₂OH (50% in water, 0.64 cm³, 10.5 mmol) in EtOH (10 cm³) were heated to reflux for 24 hours, after which the solvent and excess hydroxylamine were removed by rotary evaporator. The residue was freeze-dried and kept in high vacuum line for several hours to give 3-(2-ethoxyethoxy)-N′-hydroxypropanimidamide (1.2 g, 97.6%) as a colourless oil.

### Reaction of 3-(2-(2-(dimethylamino)ethoxy)ethoxy)propanenitrile to Give 3-(2-(2-(dimethylamino)ethoxy)ethoxy)-N′-hydroxypropanimidamide

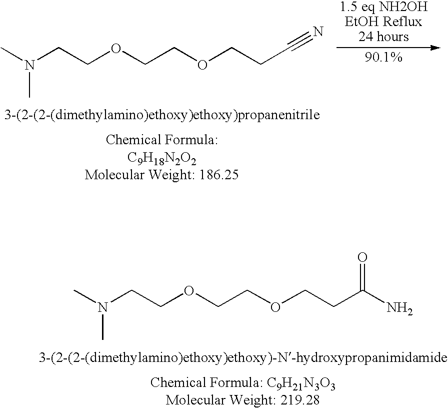

A solution of 3-(2-(2-(dimethylamino)ethoxy)ethoxy)propanenitrile (0.5 g, 2.68 mmol) and NH₂OH (50% in water, 0.25 cm³, 4 mmol) in EtOH (10 cm³) were stirred at 80° C. for 24 hours, after which the solvent and excess hydroxylamine were removed by rotary evaporator. The residue was freeze-dried and kept in high vacuum line for several hours to give 3-(2-(2-(dimethylamino)ethoxy)ethoxy)-N′-hydroxypropanimidamide (0.53 g, 90.1%) as a light yellow oil.

### Reaction of 3,3-(2,2′-(2-cyanoethylazanediyl)bis(ethane-2,1-diyl)bis(oxy))dipropanenitrile with Hydroxylamine to Give 3,3′-(2,2′-(3-amino-3-(hydroxyimino)propylazanediyl)bis(ethane-2,1-diyl))bis(oxy)bis(N′-hydroxypropanimidamide)

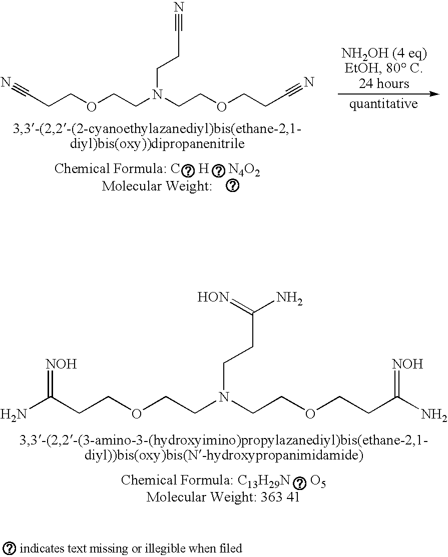

Treatment of 3,3′-(2,2′-(2-cyanoethylazanediyl)bis(ethane-2,1-diyl)bis(oxy))dipropanenitrile (0.8 g, 3 mmol) with NH₂OH (0.74 cm³, 12.1 mmol) in EtOH (8 cm³) gave 3,3′-(2,2′-(3-amino-3-(hydroxyimino)propylazanediyl)bis(ethane-2,1-diyl))bis(oxy)bis(N′-hydroxypropanimidamide) (1.09 g, 100%) as an oil.

### Reaction of Iminodipropionitrile to Give 3,3′-azanediylbis(N′-hydroxypropanimidamide)

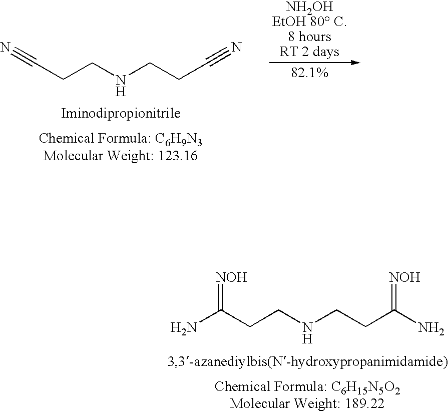

Iminodipropionitrile (1 g, 8 mmol) and hydroxylamine (50% in water, 1 cm³, 1.07 g, 16 mmol, 2 eq) in EtOH (8 cm³) were stirred at room temperature for 2 days and then at 80° C. for 8 hours. The mixture was allowed to cool and the precipitated crystals were collected by filtration and dried in high vacuum line to give the product 3,3′-azanediylbis(N′-hydroxypropanimidamide) (1.24 g 82.1%) as a white solid, mp 180° C. (lit 160° C.).

### Reaction of 3,3′,3″,3′″-(ethane-12-diylbis(azanetriyl))tetrapropanenitrile to Give 3,3′,3″,3′″-(ethane-1,2-diylbis(azanetriyl))tetrakis(N′-hydroxypropanimidamide) to Produce EDTA Analogue

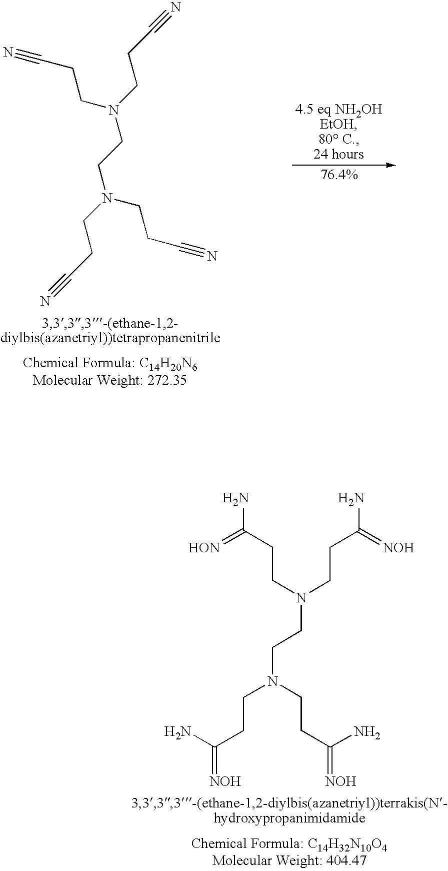

A solution of 3,3′, 3″,3′″-(ethane-1,2-diylbis(azanetriyl))tetrapropanenitrile (1 g, 4 mmol) and NH₂OH (50% in water, 1.1 cm³, 18.1 mmol) in EtOH (10 cm³) was stirred at 80° C. for 24 hours and was then allowed to cool to room temperature. The solid formed was collected by filtration and dried under vacuum to give 3,3′,3″,3′″-(ethane-1,2-diylbis(azanetriyl))tetrakis(N′-hydroxypropanimidamide) (1.17 g, 76.4%) as a white solid, mp 191-192° C.

### Reaction of 3,3′-(2,2-bis((2-cyanoethoxy)methyl)propane-1,3-diyl)bis(oxy)dipropanenitrile with Hydroxylamine to Give 3,3′-(2,2-bis((3-(hydroxyamino)-3-iminopropoxy)methyl)propane-1,3-diyl)bis(oxy)bis(N-hydroxypropanimidamide)

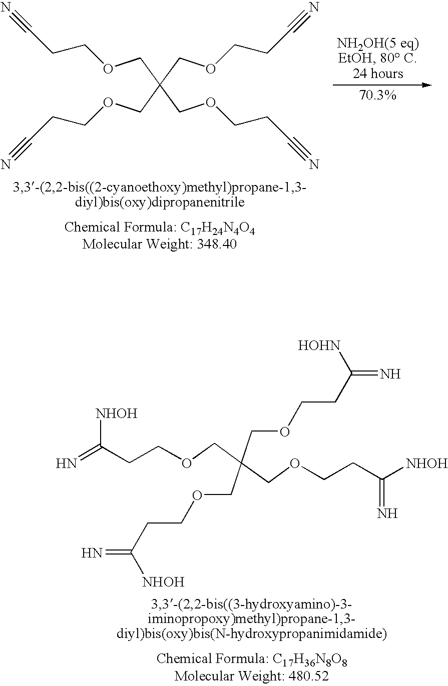

To a solution of 3,3′-(2,2-bis((2-cyanoethoxy)methyl)propane-1,3-diyl)bis(oxy)dipropanenitrile (1 g, 2.9 mmol) in EtOH (10 ml) was added NH₂OH (50% in water, 0.88 ml, 0.948 g, 14.4 mmol), the mixture was stirred at 80° C. for 24 hours and was then cooled to room temperature. Evaporation of the solvent and excess NH₂OH in the rotary evaporator followed by high vacuum for 12 hours gave 3,3′-(2,2-bis((3-(hydroxyamino)-3-iminopropoxy)methyl)propane-1,3-diyl)bis(oxy)bis(N-hydroxypropanimidamide) (0.98 g, 70.3%) as a white solid, mp 60° C.

### Reaction of 3,3′-(2-cyanophenylazanediyl)dipropanenitrile with Hydroxylamine to Give 3,3′-(2-(N′-hydroxycarbamimidoyl)phenylazanediyl)bis(N′-hydroxypropanimidamide)

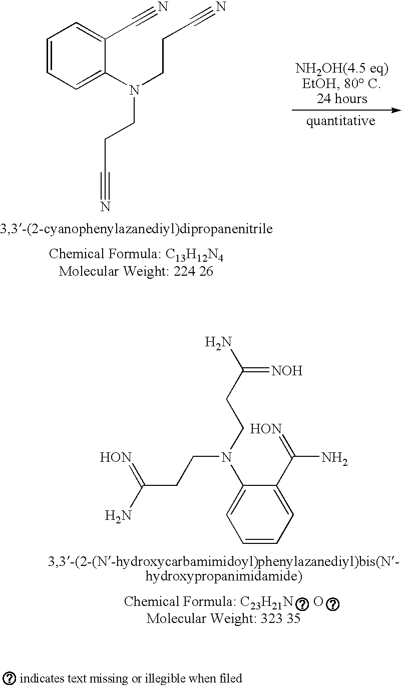

Treatment of 3,3′-(2-cyanophenylazanediyl)dipropanenitrile (1 g, 4.46 mmol) with NH2OH (1.23 ml, 20 mmol) in EtOH (10 ml) gave a crude product that was triturated with CH₂Cl₂ to give 3,3-(2-(N′-hydroxycarbamimidoyl)phenylazanediyl)bis(N′-hydroxypropanimidamide) (1.44 g, 100%) as a solid, decomposed. 81° C.

### Reaction of N,N-bis(2-cyanoethyl)acetamide with Hydroxylamine to Give N,N-bis(3-amino-3-(hydroxyimino)propyl)acetamide

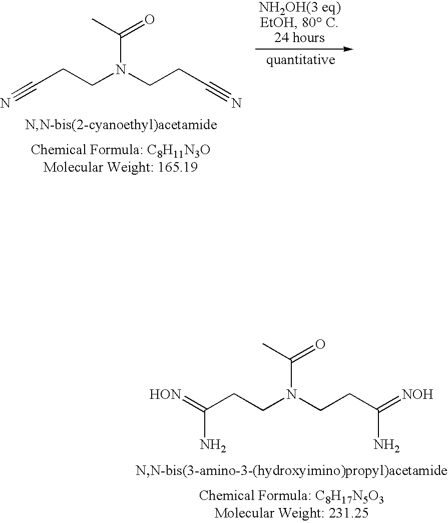

Treatment of N,N-bis(2-cyanoethyl)acetamide (0.5 g, 3.03 mmol) with NH₂OH (0.56 ml, 9.1 mmol) in EtOH (5 ml) gave N,N-bis(3-amino-3-(hydroxyimino)propyl)acetamide (0.564 g, 100%) as a white solid, mp 56.4-58° C.;

### Reaction of 3,3′-(2,2′-(methylazanediyl)bis(ethane-2,1-diyl)bis(oxy))dipropanenitrile with Hydroxylamine to Give 3,3′-(2,2′-(methylazanediyl)bis(ethane-2,1-diyl)bis(oxy))bis(N′-hydroxypropanimidamide)

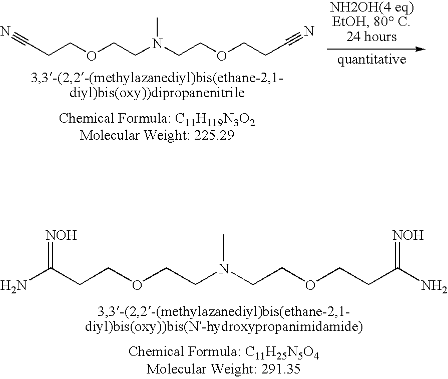

Treatment of 3,3′-(2,2′-(methylazanediyl)bis(ethane-2,1-diyl)bis(oxy))dipropanenitrile (1 g, 4.4 mmol) with NH₂OH (0.82 ml, 13.3 mmol) in EtOH (10 ml) gave 3,3′-(2,1′-(methylazanediyl)bis(ethane-2,1-diyl)bis(oxy))bis(N′-hydroxypropanimidamide) (1.28 g, 100%) as an oil.

### Reaction of Glycol Derivative 3,3′-(ethane-1,2-diylbis(oxy))dipropanenitrile to Give 3,3′-(ethane-1,2-diylbis(oxy))bis(N′-hydroxypropanimidamide)

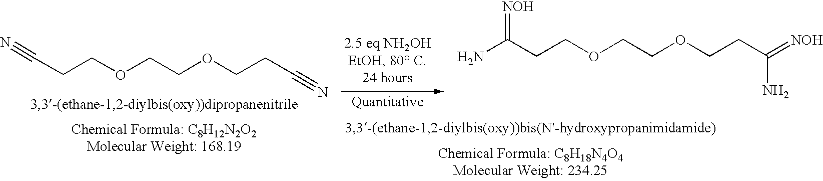

A solution of 3,3′-(ethane-1,2-diylbis(oxy))dipropanenitrile (1 g, 5 mmol) and NH₂OH (50% in water, 0.77 cm³, 12.5 mmol) in EtOH (10 cm³) was stirred at 80° C. for 24 hours and then at room temperature for 24 hours. The solvent and excess NH₂OH were evaporated off and the residue was freeze-dried to give 3,3′-(ethane-1,2-diylbis(oxy))bis(N′-hydroxypropanimidamide) (1.33 g, 100%) as a viscous oil.

### Reaction of 3,3′-(piperazine-1,4-diyl)dipropanenitrile to Give 3,3′-(piperazine-1,4-diyl)bis(N′-hydroxypropanimidamide)

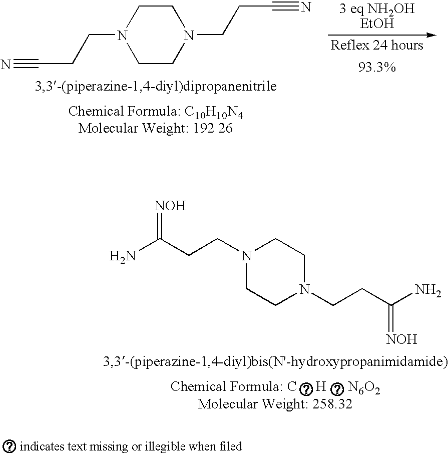

A solution of 3,3′-piperazine-1,4-diyl)dipropanenitrile (1 g, 5.2 mmol) and NH₂OH (50% in water, 0.96 cm³, 15.6 mmol) in EtOH (10 cm³) were heated to reflux for 24 hours, after which the mixture was allowed to cool to room temperature. The solid formed was collected by filtration and dried in high vacuum line to give 3,3′-(piperazine-1,4-diyl)bis(N′-hydroxypropanimidamide) (1.25 g, 93.3%) as a white solid, deep 238° C. (brown coloration at >220° C.

### Reaction of Cyanoethylated Sorbitol Compound with Hydroxylamine to Give 1,2,3,4,5,6-hexakis-O-[3-(hydroxyamino)-3-iminopropyl Hexitol

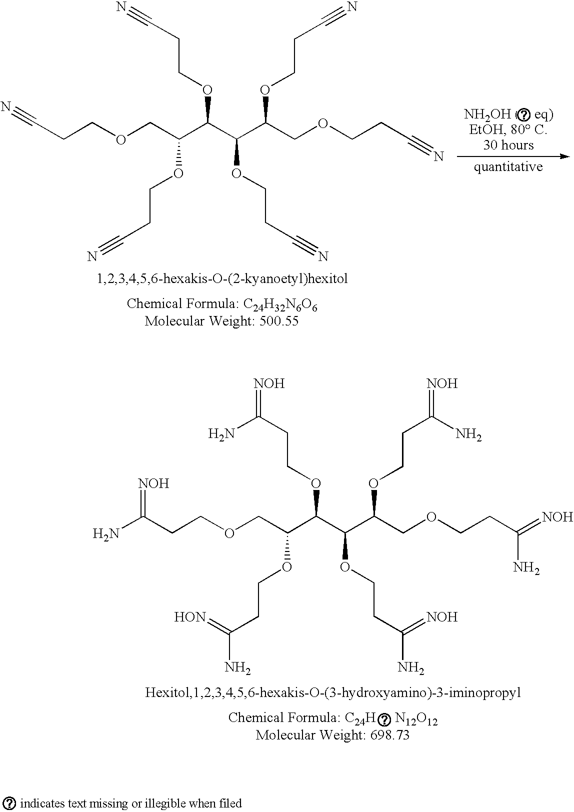

A solution of cyanoethylated product of sorbitol (0.48 g, 0.96 mmol) and NH₂OH (50% in water, 0.41 ml, 0.44 g, 6.71 mmol) in EtOH (5 ml) was stirred at 80° C. for 24 hours. Evaporation of solvent and NMR analysis of the residue showed incomplete conversion. The product was dissolved in water (10 ml) and EtOH (100 ml) and NH₂OH (0.5 go 7.6 mmol) was added. The mixture was stirred at 80° C. for a further 7 hours. Removal of all volatiles after the reaction gave 1,2,3,4,5,6-hexakis-O-[3-(hydroxyamino)-3-iminopropyl Hexitol, (0.67 g, 100%) as a white solid, mp 92-94° C. (decomposed).

### Reaction of Benzonitrile to Give N′-hydroxybenzimidamide

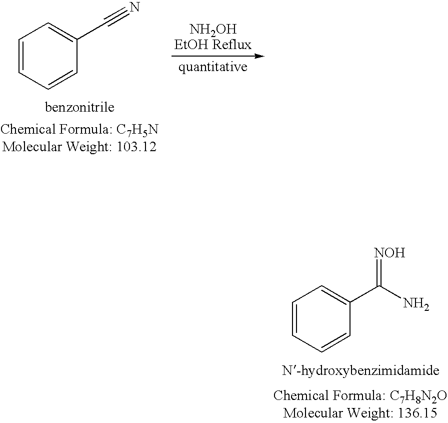

Benzonitrile (0.99 cm³, 1 g, 9.7 mmol) and hydroxylamine (50% in water, 0.89 cm³, 0.96 g, 14.55 mmol, 1.5 eq) were stirred under reflux in EtOH (10 cm³) for 48 hours. The solvent was evaporated under reduced pressure and water (10 cm³) was added to the residue. The mixture was extracted with dichloromethane (100 cm³) and the organic extract was evaporated under reduced pressure. The residue was purified by column chromatography to give the product N′-hydroxybenzimidamide (1.32 g, 100%) as a white crystalline solid, mp 79-81° C. (lit 79-80° C. This procedure is suitable for all stalling materials bearing a benzene ring.

### Reaction of 3-phenylpropionitrile to Give N′-hydroxy-3-phenylpropanimidamide

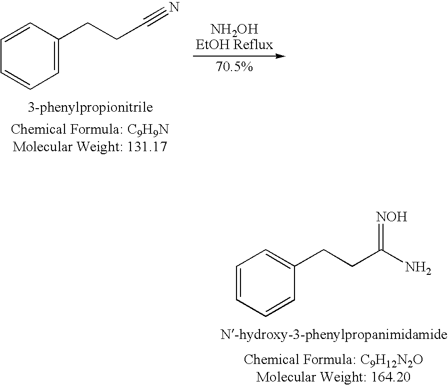

Phenylpropionitrile (1 g, 7.6 mmol) was reacted with hydroxylamine (50% in water, 0.94 cm³, 15.2 mmol, 2 eq) in EtOH (7.6 cm³) in the same manner as in the preparation of N′-hydroxybenzimidamide (EtOAc used in extraction) to give the product N′-hydroxy-3-phenylpropanimidamide (0.88 g, 70.5%) as a white solid, mp 42-43° C.

### Reaction of m-tolunitrile to Give N′-hydroxy-3-methylbenzimidamide

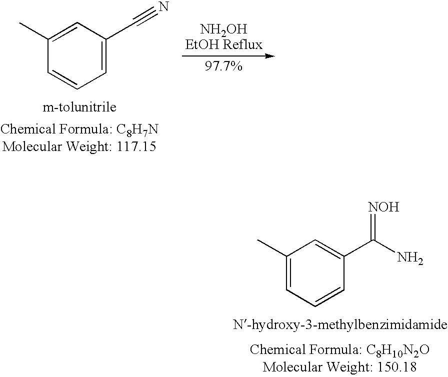

The reaction of m-tolunitrile (1 g, 8.54 mmol) and hydroxylamine (0.78 cm³, 12.8 mmol, 1.5 eq) in EtOH (8.5 cm³) was performed in the same manner as in the preparation of N′-hydroxybenzimidamide, to give the product N′-hydroxy-3-methylbenzimidamide (1.25 g, 97.7%) as a white solid, mp 92° C. (lit 88-90° C.).

### Reaction of Benzyl Cyanide to Give N′-hydroxy-2-phenylacetimidamide

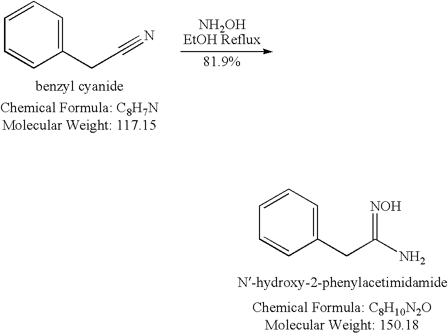

Benzyl cyanide (1 g, 8.5 mmol) and hydroxylamine (50% in water, 1.04 cm³, 17 mmol, 2 eq) in EtOH (8.5 cm³) were reacted in the same manner as in the preparation of N′-hydroxybenzimidamide (EtOAc used in extraction) to give the product N′-hydroxy-2-phenylacetimidamide (1.04 g, 81.9%) as a pale yellow solid, mp 63.5-64.5° C. (lit 57-59° C.).

### Reaction of Anthranilonitrile to Give 2-amino-N′-hydroxybenzimidamide

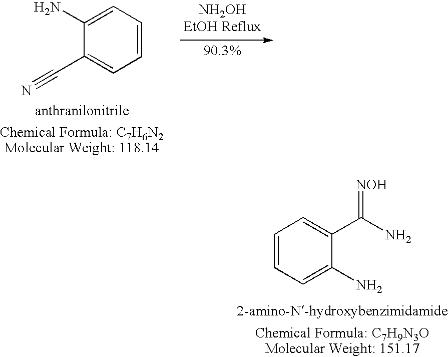

Anthranilonitrile (1 g, 8.5 mmol) and hydroxylamine (50% in water, 0.57 cm³, 9.3 mmol, 1.1 eq) in EtOH (42.5 cm³) were stirred under reflux for 24 hours, after which the volatiles were removed under reduced pressure and residue was partitioned between water (5 cm³) and CH₂Cl₂ (100 cm³). The organic phase was evaporated to dryness in the rotary evaporator followed by high vacuum line to give the product 2-amino-N′-hydroxybenzimidamide (1.16 g, 90.3%) as a solid, mp 85-86° C.

### Reaction of Phthalonitrile to Give isoindoline-1,3-dione Dioxime

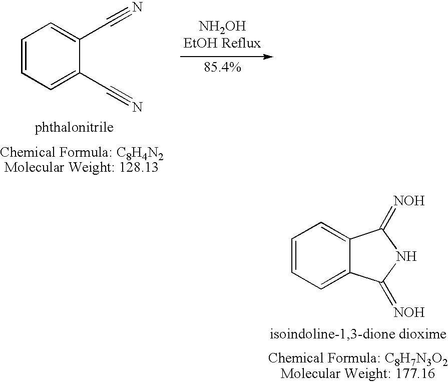

Phthalonitrile (1 g, 7.8 mmol) and hydroxylamine (1.9 cm³, 31.2 mmol, 4 eq) in EtOH (25 cm³) were stirred under reflux for 60 hours, after which the volatiles were removed under reduced pressure and the residue was washed with EtOH (2 cm³) and CH₂Cl₂ (2 cm³) to give the cyclised product isoindoline-1,3-dione dioxime (1.18 g, 85.4%) as a pale yellow solid, mp 272-275° C. (decomposed) (lit 271° C.).

### Reaction of 2-cyanophenylacetonitrile to Give the Cyclised Product 3-aminoisoquinolin-1(4H)-one Oxime or 3-(hydroxyamino)-3,4-dihydroisoquinolin-1-amine

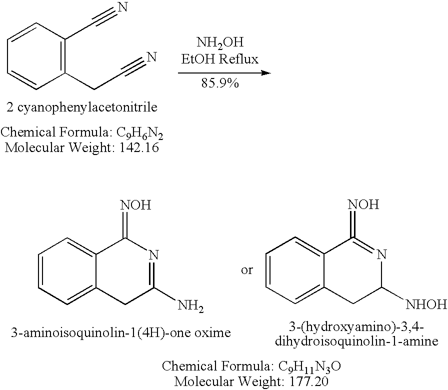

A solution of 2-cyanophenylacetonitrile (1 g, 7 mmol) and hydroxylamine (1.7 cm³, 28.1 mmol, 4 eq) in EtOH (25 cm³) were stirred under reflux for 60 hours, after which the volatiles were removed under reduced pressure. The residue was recrystallised from EtOH-water (1:4, 15 cm³) to give the cyclised product 3-aminoisoquinolin-1(4H)-one oxime or 3-(hydroxyamino)-3,4-dihydroisoquinolin-1-amine (1.15 g, 85.9%) as a solid, mp 92.5-94.5° C.

### Reaction of Cinnamonitrile to Give N′-hydroxycinnamimidamide

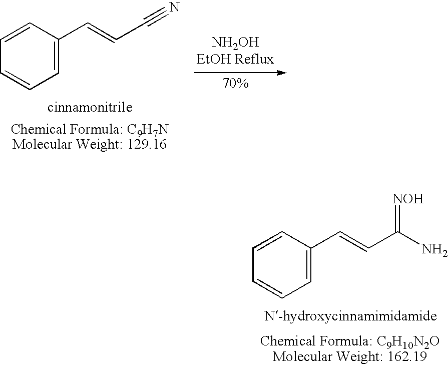

Cinnamronitrile (1 g, 7.74 mmol) and hydroxylamine (0.71 cm³, 11.6 mmol, 1.5 eq) were reacted in EtOH (7 cm³) as described for AO6 (two chromatographic separations were needed in purification) to give N′-hydroxycinnamimidamide (0.88 g, 70%) as a light orange solid, mp 85-87° C. (lit 93° C.).

### Reaction of 5-cyanophthalide to Give the Product N′-hydroxy-1-oxo-1,3-dihydroisobenzofuran-5-carboximidamide

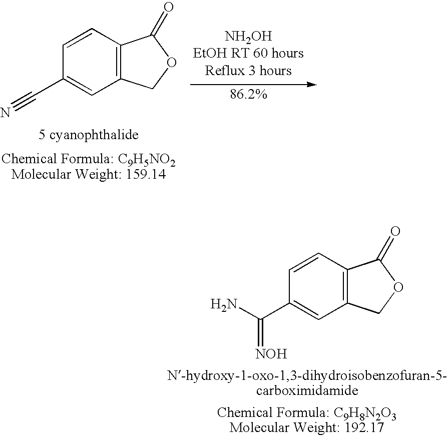

A solution of 5-cyanophthalide (1 g, 6.28 mmol) and hydroxylamine (50% in water, 0.77 cm³, 0.83 g, 12.6 mmol, 2 eq) in EtOH (50 cm³) was stirred at room temperature for 60 hours and then under reflux for 3 hours. After cooling to room temperature and standing overnight, the solid formed was collected by filtration and dried in high vacuum line to give the product N′-hydroxy-1-oxo-1,3-dihydroisobenzofuran-5-carboximidamide (1.04 g, 86.2%) as a white solid, mp 223-226° C. (decomposed).

### Reaction of 4-chlorobenzonitrile to Give the Product 4-chloro-N′-hydroxybenzimidamide

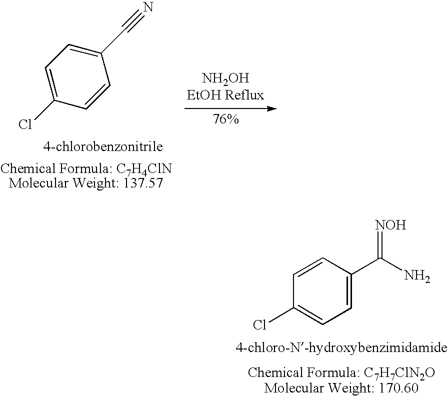

A solution of 4-chlorobenzonitrile (1 g, 7.23 mmol) and hydroxylamine (50% in water, 0.67 cm³, 10.9 mmol, 1.5 eq) in EtOH (12.5 cm³) was stirred under reflux for 48 hours. The solvent was removed under reduced pressure and the residue was washed with CH₂Cl₂ (10 cm³) to give the product 4-chloro-N′-hydroxybenzimidamide (0.94 g, 76%) as a white solid, mp 133-135° C.

### Reaction of 3-(phenylamino)propanenitrile to Give N′-hydroxy-3-(phenylamino)propanimidamide

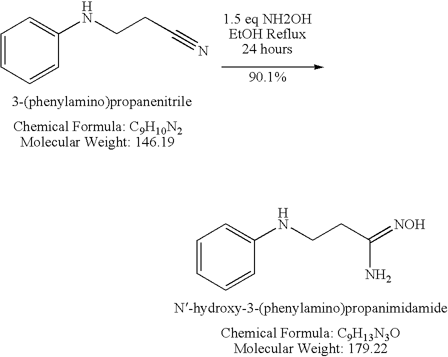

A solution of 3-(phenylamino)propanenitrile (1 g, 6.84 mmol) and NH₂OH (50% in water, 0.63 cm³, 10.26 mmol) in EtOH (10 cm³) were heated to reflux for 24 hours, after which the solvent and excess hydroxylamine were removed by rotary evaporator. To the residue was added water (10 cm³) and the mixture was extracted with CH₂Cl₂ (100 cm³). The extracts were concentrated under reduced pressure and the residue was purified by column chromatography (silica, Et₂O) to give N′-hydroxy-3-(phenylamino)propanimidamide (0.77 g, 62.8%) as a white solid, mp 93-95° C. (lit mp 91-91.5° C.).

### Reaction of 4-pyridinecarbonitrile to Give the Product N′-hydroxyisonicotinimidamide

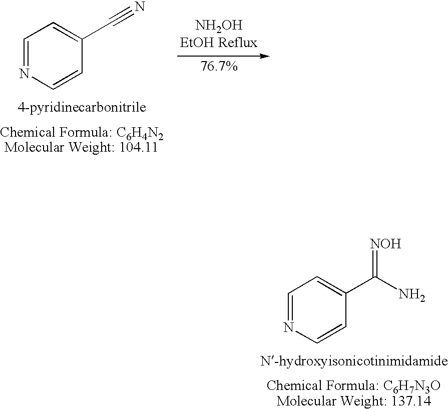

Pyridinecarbonitrile (1 g, 9.6 mmol) and hydroxylamine (50% in water, 0.88 cm³, 14.4 mmol, 1.5 eq) in EtOH (10 cm³) were stirred under reflux for 18 hours, after which the volatiles were removed under reduced pressure and the residue was recrystallised from EtOH to give the product N′-hydroxyisonicotinimidamide (1.01 g, 76.7%) as a solid, mp 203-205° C.

### Cyanoethylation of Sorbitol to Produce Multi Substituted-(2-amidoximo)ethoxy)hexane

A one-liter three-necked round-bottomed flask was equipped with a mechanical stirrer, reflux condenser, thermometer, and 100 ml addition funnel under nitrogen. Lithium hydroxide monohydrate (1.0 g, 23.8 mmol, 0.036 eq) dissolved in water (18.5 ml) was added to the flask, followed by the addition of sorbitol (120 g, 659 mmol) in one portion, and then water (100 ml). The solution was warmed to 42° C. in a water bath and treated with acrylonitrile (43.6 ml, 659 mmol, and 1.0 eq) drop-wise via the addition funnel for a period of 2 hr, while maintaining the temperature at 42° C. After the addition was complete, the solution was warmed to 50-55° C. for 4 hr and then allowed to cool to room temperature. The reaction was neutralized by addition of acetic acid (2.5 ml) and allowed to stand overnight at room temperature. The solution was evaporated under reduced pressure to give the product as a clear, viscous oil (155.4 g).

Tetramethylammonium hydroxide can be used to substitute lithium hydroxide. Elemental analysis: Found, 40.95% C; 3.85% N. The IR spectrum showed a nitrile peak at 2255 cm⁻¹ indicative of the nitrile group.

2. A one liter three-neck round-bottomed flask was equipped with a mechanical stirrer, reflux condenser, thermometer, and 100 ml addition funnel under nitrogen. Lithium hydroxide (1.0 g, 23.8 mmol, 0.036 eq) dissolved in water (18.5 ml) was added to the flask, followed by the addition of the first portion of sorbitol (60.0 g, 329 mmol) and then water (50 ml). The solution was warmed to 42° C. in a water bath and treated with acrylonitrile (42 ml, 633 mmol, 0.96 eq) drop-wise via the addition funnel for a period of 1 hr while maintaining the temperature at 42° C. The second portion of sorbitol (60 g, 329 mmol) and water (50 ml) were added to the flask. The second portion of the acrylonitrile (89.1 ml, 1.344 mol, 2.04 eq) was added in a drop-wise fashion over a period of 1 hr. After the addition was complete, the solution was warmed to 50-55° C. for 4 hr and then allowed to cool to room temperature. The reaction was neutralized by addition of acetic acid (2.5 ml) and allowed to stand overnight at room temperature. The solution was evaporated under reduced pressure to give the product as a clear, viscous oil (228.23 g).

Tetramethylammonium hydroxide can be used to substitute lithium hydroxide.

Elemental analysis: Found: 49.16% C; 10.76% N. The IR spectrum showed a nitrile peak at 2252 cm⁻¹ indicative of the nitrile group.

3. A 1000 ml 3-necked round-bottomed flask equipped with an mechanical stirrer, reflux condenser, nitrogen purge, dropping funnel, and thermometer was charged with water (18.5 ml) and lithium hydroxide monohydrate (1.75 g) and the first portion of sorbitol (44.8 g). The solution was heated to 42° C. with a water bath with stirring and the second portion of sorbitol (39.2 g) was added directly to the reaction flask. The first portion of acrylonitrile (100 ml) was then added to the reaction drop-wise via a 500 ml addition funnel over a period of 2 hr. The reaction was slightly exothermic, raising the temperature to 51° C. The final portion of sorbitol (32 g) was added for a total of 0.638 moles followed by a final portion of acrylonitrile (190 ml) over 2.5 hr keeping the reaction temperature below 60° C. (A total of 4.41 moles of acrylonitrile was used.) The reaction solution was then heated to 50-55° C. for 4 hr. The solution was then allowed to cool to room temperature and the reaction was neutralized by addition of acetic acid (2.5 ml). Removal of the solvent under reduced pressure gave the product as a clear, viscous oil (324 g).

Tetramethylammonium hydroxide can be used to substitute lithium hydroxide.

The IR spectrum showed a nitrile peak at 2251 cm⁻¹, indicative of the nitrile group.

### Preparation of (1,2,3,4,5,6-(hexa-(2-amidoximo)ethoxy)hexane.Hexitol

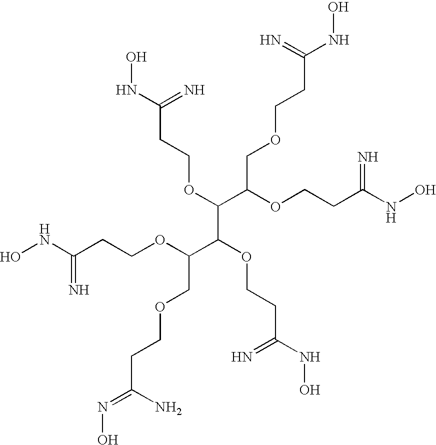

A 1000 mL three-necked round-bottomed flask was equipped with a mechanical stirrer, condenser, and addition funnel under nitrogen. CE-Sorb6 (14.77 g, 29.5 mmol) and water (200 mL) were added to the flask and stirred. In a separate 500 mL Erlenmeyer flask, hydroxylamine hydrochloride (11.47 g, 165 mmol, 5.6 eq) was dissolved in water (178 mL) and then treated with ammonium hydroxide (22.1 mL of 28% solution, 177 mmol, 6.0 eq) for a total volume of 200 mL. The hydroxylamine solution was then added in one portion directly to the mixture in the round-bottomed flask at room temperature. The stirred mixture was heated at 80° C. for 2 hr, pH=8-9, and then allowed to cool to room temperature.

Hydroxylamine freebase (50%) aqueous solution can be used to replace the solution by blending hydroxylamine chloride and ammonium hydroxide.

The IR spectrum indicated loss of most of the nitrile peak at 2250 cm⁻¹ and the appearance of a new peak at 1660 cm⁻¹, indicative of the amidoxime or hydroxamic acid.

Preparation and analysis of polyamidoxime is essentially that described in U.S. Pat. No. 3,345,344, which is incorporated herein by reference in its entirety. In that process 80 pans by weight of polyacrylonitrile of molecular weight of about 130,000 in the form of very fine powder (−300 mesh) was suspended in a solution of 300 parts by weight of hydroxylammonium sulfate, 140 parts by weight of sodium hydroxide and 2500 parts by weight of deionized water. The pH of the solution was 7.6. The mixture was heated to 90° C. and held at that temperature for 12 hours, all of the time under vigorous agitation. It was cooled to 35° C. and the product filtered off and washed repeatedly with deionized water. The resin remained insoluble throughout the reaction, but was softened somewhat by the chemical and heat. This caused it to grow from a very fine powder to small clusters of 10 to 20 mesh. The product weighed 130 grams. The yield is always considerably more than theoretical because of fumly occluded salt. The product is essentially a poly-amidoxime having the following reoccurring unit

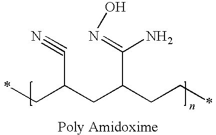

The Following Depicts Metalcomplexing Using Amidoxime Compounds.

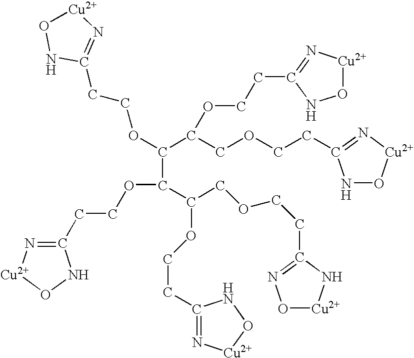

Amidoxime chelating agents can substitute for organic carboxylic acids, organic carboxylic ammonium salt or an amine carboxylates being used in cleaning formulations and processes.

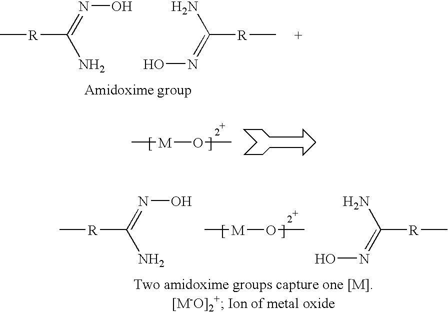

With reference to the present invention, as hereinafter more fully described, the claimed compounds can be applied to applications in the state of the art forming a background to the present invention includes the following U.S. patents, the disclosures of which hereby are incorporated herein, in their respective entireties.

### EXAMPLES OF EMBODIMENTS OF THE PRESENT INVENTION

Note that all patents cited in the examples are incorporated herein by reference regarding the proportions, amounts and support for the compositions and methods described in the examples.

### Example 1

The patents referred to in the examples herein and elsewhere in the description and summary are each hereby incorporated by reference in their entirety. One embodiment involves a method for removing organometallic and organosilicate residues remaining after a dry etch process from semiconductor substrates. The substrate is exposed to a conditioning solution of phosphoric acid, hydrofluoric acid, and a carboxylic acid, such as acetic acid, which removes the remaining dry etch residues while minimizing removal of material from desired substrate features. The approximate proportions of the conditioning solution are typically 80 to 95 percent by weight amidoxime compound and acetic acids 1 to 15 percent by weight phosphoric acid, and 0.01 to 5.0 percent by weight hydrofluoric acid. See, U.S. Pat. No. 7,261,835.

Another embodiment includes from about 0.5% to about 24% by weight of complexing agents with amidoxime functional groups with an method having a pH between about 1.5 and about 6 and comprising: at least about 75% by weight of a mixture of water and an organic solvent; from about 0.5% to about 10% by weight phosphoric acid; optionally one or more other acid compounds; optionally one or more fluoride-containing compounds; and at least one alkaline compound selected from the group consisting of: a trialkylammonium hydroxide and/or a tetraalkylammonium hydroxide; a hydroxylamine derivative; and one or more alkanolamines.

### Example 2

Table 1 lists other embodiments of the present invention where the formulations additionally include from about 0.5% to about 24% by weight of compounds with amidoxime functional groups in methods. Such formulations may contain additional components consistent with this application such as surfactants, alkaline components, and organic solvents.

TABLE 1
Examples of Useful Formulations with Chelating Agents for
Use with Amidoxime Compounds of the Present Invention

| H₃PO₄ (wt %) | Other Acid | wt % |
|---|---|---|
| 2 | methanesulfonic | 1.47 |
| 2 | pyrophosphoric acid (PPA) | 3.0 |
| 2 | Fluorosicilic | 0.24 |
| 2 | Oxalic | 2.0 |
| 4 | Oxalic | 2.0 |
| 6 | Glycolic | 1.0 |
| 3 | Oxalic | 2.0 |
| 3 | Lactic | 2.0 |
| 4 | Lactic | 2.0 |
| 3 | Citric | 2.0 |
| 4 | Citric | 2.0 |
| 3 | PPA | 0.5 |
| 3 | Glycolic | 2.0 |
| 6 | Glycolic | 2.0 |
| 3 | PPA | 2.0 |
| 3 | PPA | 4.0 |

### Example 3

Another embodiment is a composition for cleaning or etching a semiconductor substrate and method for using the same. The compositions include from about 0.01% to about 50%, more preferably about 0.5% to about 24% by weight of compounds with amidoxime functional groups may include a fluorine-containing compound as an active agent such as a quaternary ammonium fluoride, a quaternary phosphonium fluoride, sulfonium fluoride, more generally an -onium fluoride or “multi” quaternary-onium fluoride that includes two or more quaternary-onium groups linked together by one or more carbon-containing groups. The composition may further include a pH adjusting acid such as a mineral acid, carboxylic acid, dicarboxylic acid, sulfonic acid, or combination thereof to give a pH of about 2 to 9. The composition can be anhydrous and may further include an organic solvent such as an alcohol, amide, ether, or combination thereof. The composition is useful for obtaining improved etch rate, etch selectivity, etch uniformity and cleaning criteria on a variety of substrates.

### Example 4

In another embodiment, the present invention can be used with methods and compositions for removing silicon-containing sacrificial layers from Micro Electro Mechanical System (MEMS) and other semiconductor substrates having such sacrificial layers is described. The etching compositions include a supercritical fluid (SCF), an etchant species, a co-solvent, chelating agent containing at least one amidoxime group, and optionally a surfactant. Such etching compositions overcome the intrinsic deficiency of SCFs as cleaning reagents, viz., the non-polar character of SCFs and their associated inability to solubilize polar species that must be removed from the semiconductor substrate. The resultant etched substrates experience lower incidents of stiction relative to substrates etched using conventional wet etching techniques. See U.S. Pat. No. 7,160,815.

### Example 5

In another embodiment, the invention uses a supercritical fluid (SFC)-based composition, comprising at least one co-solvent, at least one etchant species, and optionally at least one surfactant, wherein said at least one etchant comprises an alkyl phosphonium difluoride and wherein said SFC-based composition is useful for etching sacrificial silicon-containing layers, said compositions containing from about 0.01% to about 50% by weight, preferably about 0.5% to about 24%, of compounds with one or more chelating group, at least one being an amidoxime functional groups. In another embodiment the surfactant comprises at least one nonionic or anionic surfactant, or a combination thereof, and the surfactant is preferably a nonionic surfactant selected from the group consisting of fluoroalkyl surfactants, polyethylene glycols, polypropylene glycols, polyethylene ethers, polypropylene glycol ethers, carboxylic acid salts, dodecylbenzenesulfonic acid; dodecylbeuzenesulfonic salts, polyaciylate polymers, dinonylphenyl polyoxyethylene, silicone polymers, modified silicone polymers, acetylenic diols, modified acetylenic diols, alkylammonium salts, modified alkylammonium salts, and combinations comprising at least one of the foregoing.

### Example 6

Another embodiment of the present invention is a composition for use in semiconductor processing wherein the composition comprises water, phosphoric acid, and an organic acid; wherein the organic acid is ascorbic acid or is an organic acid having two or more carboxylic acid groups (e.g., citric acid). The said compositions containing from about 0.01% to about 50% by weight, preferably about 0.5% to about 24%, of compounds with one or more chelating groups/agents, at least one being an amidoxime functional group/compound and such compounds can be in addition to, part of, or in substitution of the organic acid. The water can be present in about 40 wt. % to about 85 wt. % of the composition, the phosphoric acid can be present in about 0.01 wt. % to about 10 wt. % of the composition, and the organic acid can be present in about 10 wt. % to about 60 wt. % of the composition. The composition can be used for cleaning various surfaces, such as, for example, patterned metal layers and vias by exposing the surfaces to the composition. See U.S. Pat. No. 7,135,444.

### Example 7

The present invention can also be used with a polishing liquid composition for polishing a surface, with one embodiment comprising an insulating layer and a metal layer, the polishing liquid composition comprising a compound having six or more carbon atoms and a structure in which each of two or more adjacent carbon atoms has a hydroxyl group in a molecule, and water, wherein the compound having a structure in which each of two or more adjacent carbon atoms has a hydroxyl group in a molecule is represented by the formula (I): R¹—X—(CH₂)_{q}—[CH(OH)]ₙ—CH₂OH (I) wherein R¹ is a hydrocarbon group having 1 to 12 carbon atoms, X is a group represented by (CH₂)ₘ, wherein m is 1, oxygen atom, sulfur atom, COO group, OCO group, a group represented by NR² or O(R²O)P(O)O, wherein R² is hydrogen atom or a hydrocarbon group having 1 to 24 carbon atoms; q is 0 or 1; and n is an integer of 1 to 4, further comprising from about 0.01% to about 50% by weight, preferably about 0.5% to about 24%, of compounds with one or more chelating groups/agents, at least one being an amidoxime functional group/compound and such compounds can be in addition to, part of, or in substitution of an organic acid. Some embodiments include an abrasive. See U.S. Pat. No. 7,118,685.

### Example 8

Another embodiment of the present invention is a composition for use in semiconductor processing wherein the composition comprises water, phosphoric acid, and an organic acid; wherein the organic acid is ascorbic acid or is an organic acid having two or more carboxylic acid groups (e.g., citric acid), further comprising from about 0.01% to about 50% by weight, preferably about 0.5% to about 24%, of compounds with one or more chelating groups/agents, at least one being an amidoxime functional group/compound and such compounds can be in addition to, part of, or in substitution of the organic acid. The water can be present in about 40 wt. % to about 85 wt. % of the composition, the phosphoric acid can be present in about 0.01 wt. % to about 10 wt. % of the composition, and the organic acid can be present in about 10 wt, % to about 60 wt. % of the composition. The composition can be used for cleaning various surfaces, such as, for example, patterned metal layers and vias by exposing the surfaces to the composition. Sec U.S. Pat. Nos. 7,087,561, 7,067,466, and 7,029,588.

### Example 9

In another embodiment of the present invention, from about 0.01% to about 50% by weight, preferably about 0.5% to about 24%, of compounds with one or more chelating groups/agents, at least one being an amidoxime functional group/compound can be used with an oxidizing solution and process for the in situ oxidation of contaminants, including hydrocarbon, organic, bacterial, phosphonic acid, and other contaminants, the contaminants being found in various surfaces and media, including soil, sludge, and water. In a preferred embodiment, the solution further includes a peroxygen compound, such as hydrogen peroxide, in solution with a pre-mixed solution of a carboxylic acid and a halogen salt, such as glycolic acid and sodium bromide, respectively.

### Example 10

In another embodiment of the present invention from about 0.01% to about 5% by weight, preferably about 0.01 to about 0.1% of compounds with one or more chelating groups/agents, at least one being an amidoxime functional group/compound can be used with a chemical mechanical polishing slurry that is free of heteropolyacid and consisting essentially of about 3 to about 5 percent abrasive, about 3 to about 5 percent hydrogen peroxide, about 0.05 to about 0.1 percent citric acid, about 0.05 to about 0.5 percent iminodiacetic acid, about 0.005 to about 0.02 percent ammonia, and about 85-90 percent water, wherein the abrasive consists essentially of polymethylmethacrylate. See U.S. Pat. No. 7,029,373.

### Example 11

In another embodiment, the present invention includes a non-corrosive cleaning composition for removing residues from a substrate comprising: (a) water; (b) at least one hydroxyl ammonium compound; (c) at least one basic compound, preferably selected from the group consisting of amines and quaternary ammonium hydroxides; (d) at least one organic carboxylic acid; (e) from about 0.01% to about 50% by weight, preferably about 0.5% to about 24%, of compounds with one or more chelating groups/agents, at least one being an amidoxime functional group/compound and such compounds can be in addition to, part of, or in substitution of the organic acid; and (f) optionally, a polyhydric compound. The pH of the composition is preferably between about 2 to about 6. See U.S. Pat. No. 7,001,874, which is incorporated herein by reference.

### Example 12

The present invention may also be used with a cleaning solution wherein the cleaning solution also contains one of polyvalent carboxylic acid and its salt, such as where the polyvalent carboxylic acid contains at least one selected from the group consisting of oxalic acid, citric acid, malic acid, maleic acid, succinic acid, tartaric acid, and malonic acid, wherein the cleaning solution contains from about 0.01% to about 50% by weight, preferably about 0.5% to about 24%, of compounds with one or more chelating groups/agents, at least one being an amidoxime functional group/compound and such compounds can be in addition to, part of, or in substitution of the organic acid, which can be used in addition to, as part of, or in substitution of the polyvalent carboxylic acid. In another embodiment, the cleaning solution further contains a polyamino carboxylic acid and its salt. See U.S. Pat. No. 6,998,352.

### Example 13

A further embodiment of the present invention is to a method of chemically-mechanically polishing a substrate, which method comprises: (i) contacting a substrate comprising at least one layer of ruthenium and at least one layer of copper with a polishing pad and a chemical-mechanical polishing composition comprising: (a) an abrasive consisting of .alpha.-alumina treated with a negatively-charged polymer or copolymer, (b) hydrogen peroxide, (c) from about 0.01% to about 50% by weight, preferably about 0.5% to about 24% of compounds with one or more chelating groups/agents, at least one being an amidoxime functional group/compound; (d) at least one heterocyclic compound, wherein the at least one heterocyclic compound comprises at least one nitrogen atom, (e) a phosphonic acid, and (f) water, (ii) moving the polishing pad relative to the substrate, and (iii) abrading at least a portion of the substrate to polish the substrate, wherein the pH of the water and any components dissolved or suspended therein is about 6 to about 12, wherein the at least one layer of ruthenium and at least one layer of copper are in electrical contact and are in contact with the polishing composition, wherein the difference between the open circuit potential of copper and the open circuit potential of ruthenium in the water and any components dissolved or suspended therein is about 50 mV or less, and wherein a selectivity for polishing copper as compared to ruthenium is about 2 or less.

### Example 14

Another embodiment of the present invention is to a semiconductor wafer cleaning formulation, including 1-21% wt. fluoride source, 20-55% wt. organic amine(s), 0.5-40% wt. nitrogenous component, e.g., a nitrogen-containing carboxylic acid or an imine, 23-50% wt. water, and 0-21% wt. of compounds with one or more chelating groups/agents, at least one being an amidoxime functional group/compound. The formulations are useful to remove residue from wafers following a resist plasma ashing step, such as inorganic residue from semiconductor wafers containing delicate copper interconnecting structures. See U.S. Pat. No. 6,967,169.

### Example 15

The present invention also includes a method for chemical mechanical polishing copper, barter material and dielectric material, the method comprises the steps of: a) providing a first chemical mechanical polishing slurry comprising (i) 1-10 wt. % silica particles, (ii) 1-12 wt. % oxidizing agent, and (iii) 0-2 wt. % corrosion inhibitor and cleaning agent, wherein said first slurry has a higher removal rate on copper relative to a lower removal rate on said barrier material, b) chemical mechanical polishing a semiconductor wafer surface with said first slurry, c) providing a second chemical mechanical polishing slurry comprising (i) 1-10 wt. % silica particles, (ii) 0.1-1.5 wt. % oxidizing agent, and (iii) 0.1-2 wt. % carboxylic acid, having a pH in a range from about 2 to about 5, wherein the amount of (ii) is not more than the amount of (iii), and wherein said second slurry has a higher removal rate on said barrier material relative to a lower removal rate on said dielectric material and an intermediate removal rate on copper; and d) chemical mechanical polishing said semiconductor wafer surface with said second slurry, wherein either or both slurries contains from about 0.01% to about 50% by weight, preferably about 0.5% to about 24%, of compounds with one or more chelating groups/agents, at least one being an amidoxime functional group/compound. See U.S. Pat. No. 6,936,542.

### Example 16

The present invention further includes a method for cleaning a surface of a substrate, which comprises at least the following steps (1) and (2), wherein the step (2) is carried out after carrying out the step (1). Step (1): A cleaning step of cleaning the surface of the substrate with an alkaline cleaning agent containing a complexing agent, and Step (2): A cleaning step employing a cleaning agent having a hydrofluoric acid content C (wt %) of from 0.03 to 3 wt %, the complexing agent is from about 0.01% to about 50% by weight, preferably about 0.5% to about 24%, of compounds with one or more chelating groups/agents, at least one being an amidoxime functional group/compound. See U.S. Pat. No. 6,896,744.

### Example 17

Another embodiment of the present invention includes a cleaning gas that is obtained by vaporizing a carboxylic acid and/or a compound with one or more chelating groups/agents, at least one being an amidoxime functional group/compound which is supplied into a treatment chamber having an insulating substance adhering to the inside thereof, and the inside of the treatment chamber is evacuated. When the cleaning gas supplied into the treatment chamber comes in contact with the insulating substance adhering to an inside wall and a susceptor in the treatment chamber, the insulating substance is turned into a complex, so that the complex of the insulating substance is formed. The complex of the insulating substance is easily vaporized due to its high vapor pressure. The vaporized complex of the insulating substance is discharged out of the treatment chamber by the evacuation. See U.S. Pat. No. 6,893,964.

### Example 18

The present invention includes a method for rinsing metallized semiconductor substrates following treatment of the substrates with an etch residue removal chemistry, the method comprising the steps of: providing at least one metallized semiconductor substrate, the substrate having etch residue removal chemistry thereon, wherein the etch residue removal chemistry includes N-methylpyrrolidinone; rinsing the etch residue removal chemistry from the substrate and minimizing metal corrosion of the substrate by rinsing the substrate with an aqueous medium comprising an anti-corrosive agent including an organic acid selected from the group consisting of mono- and polycarboxylic acids in an amount effective to minimize metal corrosion; removing the aqueous medium from the process vessel; and introducing a drying vapor into the process vessel which the substrate remains substantially stationary within the process vessel, wherein the remover includes from about 0.01% to about 50% by weight, preferably about 0.5% to about 24%, of compounds with one or more chelating groups/agents, at least one being an amidoxime functional group/compound, which can be in addition to, part of, or in substitution of the organic acid. The composition may further include acetic acid. See U.S. Pat. No. 6,878,213.

### Example 19

The present invention may also be used with the compositions of U.S. Pat. No. 6,849,200 wherein the iminodiacetic acid component is supplemented by or substituted with compounds with one or more chelating groups/agents, at least one being an amidoxime functional group/compound.

### Example 20

The present invention also includes a method of cleaning a surface of a copper-containing material by exposing the surface to an acidic mixture comprising NO3-, F—, and one or more compounds with one or more chelating groups/agents, at least one being an amidoxime functional group/compound. The mixture may also include one or more organic acids to remove at least some of the particles. See U.S. Pat. No. 6,835,668.

### Example 21

The present invention also includes a cleaning composition comprising at least one of fluoride salts and hydrogendifluoride salts; an organic solvent having a hetero atom or atoms; optionally one or more surfactants in an amount of from 0.0001 to 10.0%; water and from about 0.01% to about 50% by weight, preferably about 0.5% to about 24%, of compounds with one or more chelating groups/agents, at least one being an amidoxime functional group/compound. See U.S. Pat. No. 6,831,048.

### Example 22

The present invention further includes a glycol-free composition for cleaning a semiconductor substrate, the composition consisting essentially of: a. an acidic buffer solution having an acid selected from a carboxylic acid and a polybasic acid and an ammonium salt of the acid in a molar ratio of acid to ammonium salt ranging from 10:1 to 1:11 and wherein the acidic buffer solution is present in an amount sufficient to maintain a pH of the composition from about 3 to about 6, b. from 30% by weight to 90% by weight of an organic polar solvent that is miscible in all proportion in water, c. from 0.1% by weight to 20% by weight of fluoride, d. from 0.5% by weight to 40% by weight of water, and e. optionally up to 15% by weight of a corrosion inhibitor. The composition further contains from about 0.01% to about 50% by weight, preferably about 0.5% to about 24%, of compounds with one or more chelating groups/agents, at least one being an amidoxime functional group/compound or such compounds may be used in place of the corrosion inhibitor. See U.S. Pat. No. 6,828,289.

### Example 23

The present invention further includes compositions containing AEEA and or AEEA derivatives which can be present in an amount ranging from about 1% to about 99%, though in most instances the amount ranges from about 10% to about 85%. For each AEEA range given for various compositions described herein, there is a “high-AEEA” embodiment where the amount of AEEA is in the upper half of the range, and a “low-AEEA” embodiment where AEEA is present in an amount bounded by the lower half of the range. Generally, the higher AEEA embodiments exhibit lower etch rates than the low AEEA embodiments for selected substrates, the embodiments further include from about 0.01% to about 50% by weight, preferably about 0.5% to about 24%, of compounds with one or more chelating groups/agents, at least one being an amidoxime functional group/compound. In most embodiments, these compositions also include other compounds, particularly polar organic solvents, water, alkanolamines, hydroxylamines, additional chelating agents, and/or corrosion inhibitors. See U.S. Pat. No. 6,825,156.

### Example 24

A composition for the stripping of photoresist and the cleaning of residues from substrates, and for silicon oxide etch, comprising from about 0.01 percent by weight to about 10 percent by weight of one or more fluoride compounds, from about 10 percent by weight to about 95% by weight of a sulfoxide or sulfone solvent, and from about 20 percent by weight to about 50 percent by weight water, further including from about 0.01% to about 50% by weight, preferably about 0.5% to about 24%, of compounds with one or more chelating groups/agents, at least one being an amidoxime functional group/compound. The composition may contain corrosion inhibitors, chelating agents, co-solvents, basic amine compounds, surfactants, acids and bases. See U.S. Pat. No. 6,777,380.

### Example 25

A polishing composition for polishing a semiconductor substrate has a pH of under 5.0 and comprises (a) a carboxylic acid polymer comprising polymerized unsaturated carboxylic acid monomers having a number average molecular weight of about 20,000 to 1,500,000 or blends of high and low number average molecular weight polymers of polymerized unsaturated carboxylic acid monomers, (b) 1 to 15% by weight of an oxidizing agent, (c) up to 3.0% by weight of abrasive particles, (d) 50-5,000 ppm (parts per million) of an inhibitor, (e) up to 3.0% by weight of a complexing agent, such as, malic acid, and (f) 0.1 to 5.0% by weight of a surfactant, from about 0.01% to about 50% by weight, preferably about 0.5% to about 24%, of compounds with one or more chelating groups/agents, at least one being an amidoxime functional group/compound. See U.S. Pat. No. 6,679,928.

### Example 26

Particulate and metal ion contamination is removed from a surface, such as a semiconductor wafer containing copper damascene or dual damascene features, employing aqueous composition comprising a fluoride containing compound; a dicarboxylic acid and/or salt thereof; and a hydroxycarboxylic acid and/or salt thereof, the composition contains from about 0.01% to about 50% by weight, preferably about 0.5% to about 24%, of compounds with one or more chelating groups/agents, at least one being an amidoxime functional group/compound. See U.S. Pat. No. 6,673,757.

### Example 27

A semiconductor wafer cleaning formulation, including 2-98% wt. organic amine, 0-50% wt. water, 0.1-60% wt. 1,3-dicarbonyl compound chelating agent, 0-25% wt. of additional different chelating agent(s), 0.540% wt. nitrogen-containing carboxylic acid or an imine, and 2-98% wt polar organic solvent. The formulations are useful to remove residue from wafers following a resist plasma ashing step, such as inorganic residue from semiconductor wafers containing delicate copper interconnecting structures.

### Example 28

Another embodiment of the present invention relates to a method useful in removing etch residue from etcher equipment parts. The compositions used are aqueous, acidic compositions containing fluoride and polar, organic solvents. The compositions are free of glycols and hydroxyl amine and have a low surface tension and viscosity and further include from about 0.01% to about 50% by weight, preferably about 0.5% to about 24%, of compounds with one or more chelating groups/agents, at least one being an amidoxime functional group/compound. See U.S. Pat. No. 6,656,894.

### Example 29

The invention includes a method of cleaning a surface of a copper-containing material by exposing the surface to an acidic mixture comprising NO³—, F— and from about 0.01% to about 50% by weight, preferably about 0.5% to about 24%, of compounds with one or more chelating groups/agents, at least one being an amidoxime functional group/compound and/or one or more organic acid anions having carboxylate groups. The invention also includes an improved semiconductor processing method of forming an opening to a copper-containing material. A mass is formed over a copper-containing material within an opening in a substrate. The mass contains at least one of an oxide barrier material and a dielectric material. A second opening is etched through the mass into the copper-containing material to form a base surface of the copper-containing material that is at least partially covered by particles comprising at least one of a copper oxide, a silicon oxide or a copper fluoride. The base surface is cleaned with a solution comprising nitric acid, hydrofluoric acid and one or more organic acids to remove at least some of the particles.

One or more organic acids may be used in the composition of this example. An exemplary composition includes an acetic acid solution (99.8%, by weight in water), an HF solution (49%, by weight in water), an HNO₃ solution (70.4%, by weight in water), and H₂O the resulting cleaning mixture being: from about 3% to about 20% compounds with one or more chelating groups/agents, at least one being an amidoxime functional group/compound, by weight; from about 0.1% to about 2.0% HNO₃ by weight; and from about 0.05% to about 3.0% HF, by weight. See U.S. Pat. No. 6,589,882.

### Example 30

Another embodiment of the present invention is a composition for selective etching of oxides over a metal. The composition contains water, hydroxylammonium salt, one or more compounds with one or more chelating groups/agents, at least one being an amidoxime functional group/compound, a fluorine containing compound, and optionally, a base. The pH of the composition is about 2 to 6. See U.S. Pat. No. 6,589,439.

### Example 31

Another embodiment of the present invention is an etching treatment comprising a combination including hydrofluoric acid of 15 percent by weight to 19 percent by weight, one or more compounds with one or more chelating groups/agents, at least one being an amidoxime functional group/compound of 0.5 percent by weight to 24 percent by weight and ammonium fluoride of 12 percent by weight to 42 percent by weight, said combination having a hydrogen ion concentration of 10⁻⁶ mol/L to 10^{-1.8}, further comprising a surfactant of 0.001 percent by weight to 1 percent by weight. See U.S. Pat. No. 6,585,910.

### Example 32

Another embodiment of the present invention includes a semiconductor wafer cleaning formulation, including 2-98% wt. organic amine, 0-50% wt. water, 0.1-60% wt. one or more compounds with one or more chelating groups/agents, at least one being an amidoxime functional group/compound, 0-25% wt. of additional different chelating agent(s), 0.1-40% wt. nitrogen-containing carboxylic acid or an imine, optionally 1,3-dicarbonyl compound chelating agent, and 2-98% wt polar organic solvent. The formulations are useful to remove residue from wafers following a resist plasma ashing step, such as inorganic residue from semiconductor wafers containing delicate copper interconnecting structures. See U.S. Pat. No. 6,566,315.

### Example 33

An alternative embodiment of the present invention is a method for removing organometallic and organosilicate residues remaining after a dry etch process from semiconductor substrates. The substrate is exposed to a conditioning solution of a fluorine source, a non-aqueous solvent, a complementary acid, and a surface passivation agent. The fluorine source is typically hydrofluoric acid. The non-aqueous solvent is typically a polyhydric alcohol such as propylene glycol. The complementary acid is typically either phosphoric acid or hydrochloric acid. The surface passivation agent is one or more compounds with one or more chelating groups/agents, at least one being an amidoxime functional group/compound, and may optionally include a carboxylic acid such as citric acid. Exposing the substrate to the conditioning solution removes the remaining dry etch residues while minimizing removal of material from desired substrate features. See U.S. Pat. No. 6,562,726.

### Example 34

Another embodiment of the present invention is a stripping and cleaning composition for the removal of residue from metal and dielectric surfaces in the manufacture of semi-conductors and microcircuits. The composition is an aqueous system including organic polar solvents including corrosive inhibitor component from one or more compounds with one or more chelating groups/agents, at least one being an amidoxime functional group/compound and optionally a select group of aromatic carboxylic acids used in effective inhibiting amounts. A method in accordance with this invention for the removal of residues from metal and dielectric surfaces comprises the steps of contacting the metal or dielectric surface with the above inhibited compositions for a time sufficient to remove the residues. See U.S. Pat. No. 6,558,879.

### Example 35

Another embodiment of the present invention is a homogeneous non-aqueous composition containing a fluorinated solvent, ozone, one or more compounds with one or more chelating groups/agents, at least one being an amidoxime functional group/compound, and optionally a co-solvent and the use of these compositions for cleaning and oxidizing substrates is described. See U.S. Pat. No. 6,537,380.

### Example 36

The present invention also includes a chemical mechanical polishing slurry and method for using the slurry for polishing copper, barrier material and dielectric material that comprises a first and second slurry. The first slurry has a high removal rate on copper and a low removal rate on barrier material. The second slurry has a high removal rate on barrier material and a low removal rate on copper and dielectric material. The first and second slurries at least comprise silica particles, an oxidizing agent, one or more compounds with one or more chelating groups/agents, at least one being an amidoxime functional group/compound, optionally a corrosion inhibitor, and a cleaning agent. See, U.S. Pat. No. 6,527,819.

### Example 37

Another embodiment of the present invention also includes a method for removing organometallic and organosilicate residues remaining after a dry etch process from semiconductor substrates. The substrate is exposed to a conditioning solution of phosphoric acid, hydrofluoric acid, and one or more compounds with one or more chelating groups/agents, at least one being an amidoxime functional group/compound, and optionally a carboxylic acid, such as acetic acid, which removes the remaining dry etch residues while minimizing removal of material from desired substrate features. The approximate proportions of the conditioning solution are typically 80 to 95 percent by weight one or more compounds with one or more chelating groups/agents, at least one being an amidoxime functional group/compound and carboxylic acid, 1 to 15 percent by weight phosphoric acid, and 0.01 to 5.0 percent by weight hydrofluoric acid. U.S. Pat. No. 6,517,738.

### Example 38

Another embodiment of the present invention is a composition for use in semiconductor processing wherein the composition comprises water, phosphoric acid, and one or more compounds with one or more chelating groups/agents, at least one being an amidoxime functional group/compound, and optionally an organic acid; wherein the organic acid is ascorbic acid or is an organic acid having two or more carboxylic acid groups (e.g., citric acid). The water can be present in about 40 wt. % to about 85 wt. % of the composition, the phosphoric acid can be present in about 0.01 wt. % to about 10 wt. % of the composition, and the one or more compounds with one or more chelating groups/agents, at least one being an amidoxime functional group/compound and organic acid can be present in about 10 wt. % to about 60 wt. % of the composition. The composition can be used for cleaning various surfaces, such as, for example, patterned metal layers and vias by exposing the surfaces to the composition. See U.S. Pat. No. 6,486,108.

### Example 39

Another embodiment of the present invention is a method for removing organometallic and organosilicate residues remaining after a dry etch process from semiconductor substrates. The substrate is exposed to a conditioning solution of phosphoric acid, hydrofluoric acid, and one or more compounds with one or more chelating groups/agents, at least one being an amidoxime functional group/compound, and optionally a carboxylic acid, such as acetic acid, which removes the remaining dry etch residues while minimizing removal of material from desired substrate features. The approximate proportions of the conditioning solution are typically 80 to 95 percent by weight one or more compounds with one or more chelating groups/agents, at least one being an amidoxime functional group/compound and acetic acid, 1 to 15 percent by weight phosphoric acid, and 0.01 to 5.0 percent by weight hydrofluoric acid. See U.S. Pat. No. 6,453,914.

### Example 40

Another example of the present invention is show in cleaning a substrate which has a metal material and a semiconductor material both exposed at the surface and which has been subjected to a chemical mechanical polishing treatment, the substrate is first cleaned with a first cleaning solution containing ammonia water, etc. and then with a second cleaning solution containing (a) a first complexing agent capable of easily forming a complex with the oxide of said metal material, etc. and (b) an anionic or cationic surfactant. See U.S. Pat. No. 6,444,583.

### Example 41

The present invention is also exemplified by a cleaning agent for semiconductor parts, which can decrease a load on the environment and has a high cleaning effect on CMP (chemical mechanical polishing) abrasive particles, metallic impurities and other impurities left on the semiconductor parts such as semiconductor substrates after the CMP, comprising a (copolymer having one or more compounds with one or more chelating groups/agents, at least one being an amidoxime functional group/compound, and optionally at least one kind of group selected from the group consisting of sulfonic acid salt) groups and carboxylic acid (salt) groups, the cleaning agent further containing a phosphonic acid (salt) group-containing (co)polymer, a phosphonic acid compound or a surfactant as needed; and a method for cleaning semiconductor parts with the above cleaning agent. See U.S. Pat. No. 6,440,856.

### Example 42

The present invention also includes a non-corrosive cleaning composition for removing residues from a substrate. The composition comprises: (a) water; (b) at least one hydroxylammonium compound; (c) at least one basic compound, preferably selected from the group consisting of amines and quaternary ammonium hydroxides; (d) one or more compounds with one or more chelating groups/agents, at least one being an amidoxime functional group/compound, (e) optionally at least one organic carboxylic acid; and (f) optionally, a polyhydric compound. The pH of the composition is preferably between about 2 to about 6. See U.S. Pat. No. 6,413,923.

### Example 43

Another embodiment of the present invention is a composition comprising a slurry having an acidic pH and a corrosion inhibitor with one or more compounds with one or more chelating groups/agents, at least one being an amidoxime functional group/compound, and optionally a carboxylic acid corrosion inhibitor, wherein said carboxylic acid is selected from the group consisting of: glycine, oxalic acid, malonic acid, succinic acid and nitrilotriacetic acid. U.S. Pat. No. 6,409,781.

### Example 44

An alternative embodiment of the present invention is a chemical formulation consisting of a chelating agent, wherein said chelating agent is one or more compounds with one or more chelating groups/agents, at least one being an amidoxime functional group/compound, and optionally one or more additional chelating agents selected from the group consisting of iminodiacetic, malonic, oxalic, succinic, boric and malic acids and 2,4 pentanedione; a fluoride; and a glycol solvent, wherein said chelating agents consist of approximately 0.1-10% by weight of the formulation, and wherein said fluoride consists of a compound selected from the group consisting of ammonium fluoride, an organic derivative of ammonium fluoride, and a organic derivative of a polyammonium fluoride; and wherein said fluoride consists of approximately 1.65-7% by weight of the formulation; and wherein said glycol solvent consists of approximately 73-98.25% by weight of said formulation, further comprising: an amine, wherein said amine consists of approximately 0.1-10% by weight of said formulation. The chelating agents generally contain one or more compounds with one or more chelating groups/agents, at least one being an amidoxime functional group/compound, and optionally contain two carboxylic acid groups or two hydroxyl groups or two carbonyl groups such that the two groups in the chelating agent are in close proximity to each other. Other chelating agents which are also weakly to moderately acidic and are structurally similar to those claimed are also expected to be suitable. See U.S. Pat. No. 6,383,410.

### Example 45

Another embodiment of the present invention is a cleaning composition comprising a partially fluorinated solvent, a co-solvent, one or more compounds with one or more chelating groups/agents, at least one being an amidoxime functional group/compound, and ozone, wherein said co-solvent is selected from the group consisting of ethers, esters, tertiary alcohols, carboxylic acids, ketones and aliphatic hydrocarbons. See U.S. Pat. No. 6,372,700.

### Example 46

Yet another embodiment of the present invention is a combination of one or more compounds with one or more chelating groups/agents, at least one being an amidoxime functional group/compound and optionally a carboxylic acid corrosion inhibitor. The combination of corrosion inhibitors can effectively inhibit meta corrosion of aluminum, copper, and their alloys. Suitable carboxylic acids include monocarboxylic and polycarboxylic acids. For example, the carboxylic acid may be, but is not limited to, formic acid, acetic acid, propionic acid, valeric acid, isovaleric acid, oxalic acid, malonic acid, succinic acid, glutaric acid, maleic acid, fimaric acid, phthalic acid, 1,2,3-benzenetricarboxylic acid, glycolic acid, lactic acid, citric acid, salicylic acid, tartaric acid, gluconic acid, and mixtures thereof. The preferred carboxylic acid is citric acid.

### Example 47

Another example of the present invention is a composition for selective etching of oxides over a metal comprising: (a) water; (b) hydroxylammonium salt in an amount about 0.1 wt. % to about 0.5 wt. % of said composition; (c) one or more compounds with one or more chelating groups/agents, at least one being an amidoxime functional group/compound; (d) optionally a carboxylic acid selected from the group consisting of: formic acid, acetic acid, propionic acid, valeric acid, isovaleric acid, oxalic acid, malonic acid, succinic acid, glutaric acid, maleic acid, fimaric acid, phthalic acid, 1,2,3-benzenetricarboxylic acid, glycolic acid, lactic acid, citric acid, salicylic acid, tartaric acid, gluconic acid, and mixtures thereof; (e) a fluorine-containing compound; and (e) optionally, base. See U.S. Pat. No. 6,361,712.

### Example 48

In a further aspect, the invention relates to a semiconductor wafer cleaning formulation for use in post plasma ashing semiconductor fabrication, comprising the following components in the percentage by weight (based on the total weight of the formulation) ranges shown:

| | | |
|---|---|---|
| Organic amine(s) | 2-98% | by weight |
| Water | 0-50% | by weight |
| amidoxime chelating agent | O.1-60% | by weight |
| Complexing agent | 0-25% | by weight |
| Nitrogen-containing carboxylic acid or imine | 0.5-40% | by weight |
| polar organic solvent | 2-98% | by weight. |

### Example 49

Another example of the present invention includes an essentially anhydrous cleaning composition comprising 88 weight percent or more of a fluorinated solvent, from 0.005 to 2 weight percent of hydrogen fluoride or complex thereof, and from 0.01 to 5 weight percent of a co-solvent, wherein said cosolvent is selected from one or more compounds with one or more chelating groups/agents, at least one being an amidoxime functional group/compound, ethers, polyethers, carboxylic acids, primary and secondary alcohols, phenolic alcohols, ketones, aliphatic hydrocarbons and aromatic hydrocarbons. See U.S. Pat. No. 6,310,018.

### Example 50

| | | | |
|---|---|---|---|
| A. | Amidoxime compound | 2.5% | by weight |
| | Tetramethylammonium fluoride | 4.5% | by weight |
| | Ethylene glycol | 93% | by weight |
| B. | Amidoxime compound | 1.3% | by weight |
| | Pentamethyldiethylenetriammonium | 4.6% | by weight |
| | trifluoride | | |
| | Ethylene glycol | 94.1% | by weight |
| C. | Amidoxime compound | 1.25% | by weight |
| | Triethanolammonium fluoride | 5% | by weight |
| | Ethylene glycol | 93.75% | by weight |
| D. | Amidoxime compound | 2.8% | by weight |
| | Tetramethylammonium fluoride | 5.1% | by weight |
| | Ethylene glycol | 92.1% | by weight |
| E. | Amidoxime compound | 2% | by weight |
| | Ammonium fluoride | 7% | by weight |
| | Ethylene glycol | 91% | by weight |
| F. | Amidoxime compound | 2.8% | by weight |
| | Ammonium fluoride | 5% | by weight |
| | Ethylene glycol | 92.2% | by weight |

### Example 51

Another embodiment of the present invention includes a chelating agent, a fluoride salt, and a glycol solvent, wherein said chelating agent is weakly to moderately acidic, and consists of approximately 0.1-10% by weight of the formulation; and wherein said fluoride salt consists of a compound selected from the group consisting of ammonium fluoride, an organic derivative of ammonium fluoride, and a organic derivative of a polyammonium fluoride; and wherein said fluoride salt consists of approximately 1.65-7% by weight of the formulation; and wherein said glycol solvent consists of 73-98.25% by weight of said formulation; and further including an amine, wherein said amine consists of approximately 0.1-10% by weight of said formulation; and wherein said chelating agent is an amidoxime or hydroxamic acid. See U.S. Pat. No. 6,280,651.

### Example 52

Another example of the present invention is a cleaning agent for use in producing semiconductor devices, which consists essentially of an aqueous solution containing (A) 0.1 to 15% by weight based on the total amount of the cleaning agent of at least one fluorine-containing compound selected from the group consisting of hydrofluoric acid, ammonium fluoride, ammonium hydrogenfluoride, acidic ammonium fluoride, methylamine salt of hydrogen fluoride, ethylamine salt of hydrogen fluoride, propylamine salt of hydrogen fluoride and tetramethylammonium fluoride, (B) 0.1 to 15% by weight based on the total amount of the cleaning agent of a salt of boric acid and (C) 0.5 to 50% by weight of one or more compounds with one or more chelating groups/agents, at least one being an amidoxime functional group/compound; and (d) 5 to 80% by weight based on the total amount of the cleaning agent of a water-soluble organic solvent, and optionally further containing at least one of a quaternary ammonium salt, an ammonium salt of an organic carboxylic acid, an amine salt of an organic carboxylic acid and a surfactant. See U.S. Pat. No. 6,265,309.

### Example 53

Another embodiment of the present invention includes a cleaning liquid in the form of an aqueous solution for cleaning a semiconductor device during production of a semiconductor device, which comprises (A) a fluorine-containing compound; (B) a water-soluble or water-miscible organic solvent; (C) one or more compounds with one or more chelating groups/agents, at least one being an amidoxime functional group/compound; (D) optionally, an organic acid; and (E) a quaternary ammonium salt. In some embodiments the cleaning solution also contains a surfactant. The organic acid is typically selected from the group consisting of formic acid, acetic acid, propionic acid, butyric acid, isobutyric acid, valeric acid, isovaleric acid, heptanoic acid, lauric acid, palmitic acid, stearic acid, acrylic acid, crotonic acid, methacrylic acid, oxalic acid, malonic acid, maleic acid, succinic acid, adipic acid, azelaic acid, sebacic acid, benzoic acid, toluic acid, phthalic acid, trimellitic acid, pyromellitic acid, benzenesulfonic acid, toluenesulfonic acid, salicylic acid and phthalic anhydride. See U.S. Pat. No. 5,972,862.

### Example 54

Another embodiment is a method for semiconductor processing comprising etching of oxide layers, especially etching thick SiO2 layers and/or last step in the cleaning process wherein the oxide layers are etched in the gas phase with a mixture of hydrogen fluoride, one or more compounds with one or more chelating groups/agents, at least one being an amidoxime functional group/compound, and optionally one or more carboxylic acids, eventually in admixture with water. See U.S. Pat. No. 5,922,624.

### Example 55

The complexing agents of the present invention may also be added to the rinse containing a peroxide of U.S. Pat. No. 5,911,836.

### Example 56

(CMP) pad after performing a CMP operation on a wafer, the CMP pad having a residue on a surface of the CMP pad, the method comprising: applying chemicals onto the surface of the CMP pad; rinsing the pad surface to substantially remove by-product produced by the chemicals; and performing a mechanical conditioning operation on the surface of the pad, wherein during the CMP operation the wafer surface includes copper and oxide wherein when the wafer surface contains more copper than the oxide, the chemicals are selected from one or a combination of amidoxime and deionized water; and amidoxime+H₂O₂+deionized water amidoxime+hydroxylamine+deionized water.

### Example 57

Another embodiment of the present invention includes a cleaning solution contains DI water, amidoxime, hydrogen peroxide (H₂O₂), and DI water. The concentration of amidoxime is preferably about 1% by weight. The mixing ratio of amidoxime:H₂O₂:DI water is preferably about 1:4:20 by volume, and most preferably about 1:1:5. The waiting time for allowing this solution to react with the residue is preferably between about 30 and about 180 seconds, and most preferably about 60 seconds. This solution may also be applied to the polishing pad at a heated temperature that is preferably between about 40 and about 80 degrees Celsius., and most preferably about 60° C.

### Example 58

Another example of the present invention is a method and apparatus for increasing the deposition of ions onto a surface, such as the adsorption of uranium ions on the detecting surface of a radionuclide detector. The method includes the step of exposing the surface to one or more compounds with one or more chelating groups/agents, at least one being an amidoxime functional group/compound, and optionally, a phosphate ion solution, which has an affinity for the dissolved species to be deposited on the surface. This provides, for example, enhanced sensitivity of the radionuclide detector. See U.S. Pat. No. 5,652,013.

### Example 59

Copper blanket wafer is immersed in the following solutions at room temperature for 15 and 30 minutes to observe the copper thickness changes. The amidoxime compound is 1,2,3,4,5,6-hexakis-O-[3-(hydroxyamino)-3-iminopropyl Hexitol.

| | | H₂O₂ | H₂O₂/AO | AO |
|---|---|---|---|---|
| Hydrogen Peroxide | | 3% | 3% | 0 |
| 1,2,3,4,5,6-hexakis- | | 0 | 1% | 1% |
| O-[3-(hydroxyamino)- | | | | |
| 3-iminopropyl Hexitol | | | | |
| Water | | Balance | Balance | Balance |
| Copper | 15 minutes | 97 Å | 16 Å | 22 Å |
| Thickness | 30 minutes | 120 Å | 13 Å | 48 Å |
| Lost | | | | |

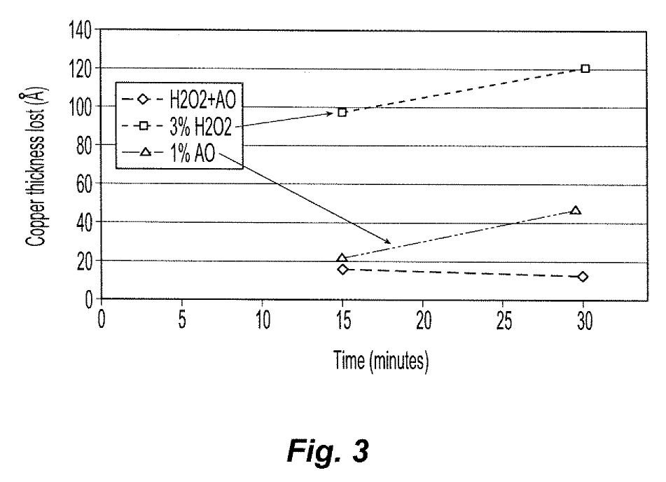

Hydrogen peroxide attacked the copper surface and changed into copper oxide resulting in the reduction of copper thickness. It resulted in a loss of 120 Å in 30 minutes of immersion. Amidoxime etches copper slightly in 30 minutes to remove about 50 Å. It unexpectedly appears that the mixture of the two components inhibits the oxidation of the copper surface. FIG. 3 shows a plot of copper thickness lost vs. time.

### Example 60

Copper blanket wafer is immersed in the following solutions at room temperature for 30 minutes at various temperatures to observe the copper thickness changes.

| | | Hydroxylamine | Hydroxylamine | |
|---|---|---|---|---|
| | | (50%) | (50%)/AO | AO |
| Hydroxylamine (50%) | | 10% | 10% | 0 |
| 1,2,3,4,5,6-hexakis- | | 0 | 10% | 10% |
| O-[3-(hydroxyamino)- | | | | |
| 3-iminopropyl Hexitol | | | | |
| Water | | Balance | Balance | Balance |
| Copper | RT | 2.88 | 9.76 | 4.08 |
| Thickness | 40° C. | 5.27 | 32.68 | 5.83 |
| Lost | 60° C. | 7.95 | 61.68 | 4.39 |
| Å/min | | | | |

The copper static etch rate, when 1,2,3,4,56-hexakis-O-[3-(hydroxyamino)-3-iminopropyl Hexitol is mixed with hydroxylamine (50%) increases exponentially from about 8 Å/min to 62 Å/min. This indicates the combination of amidoxime compound with hydroxylamine to improve copper and copper oxide debris removal from the CMP pad.

### Example 61

A sample coupon of the electroplated copper wafer is immersed in 10% of amidoxime in water at 30 C for 30 minutes. The sample is then rinsed in DI water for 5 minutes and blew dried with nitrogen gas. The sample was then sent to Evan Laboratory for ESCA and Auger analysis.

The sample was then re-analyzed again after 10 days of exposure to normal storage condition.

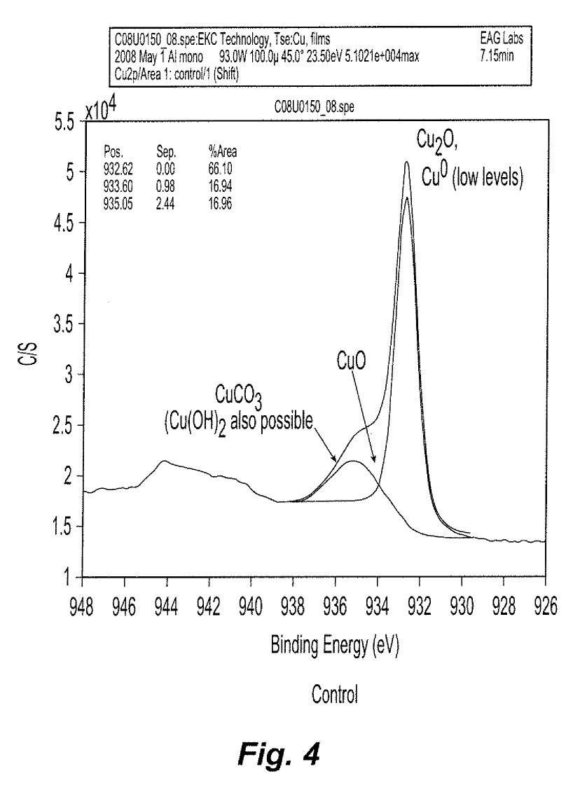

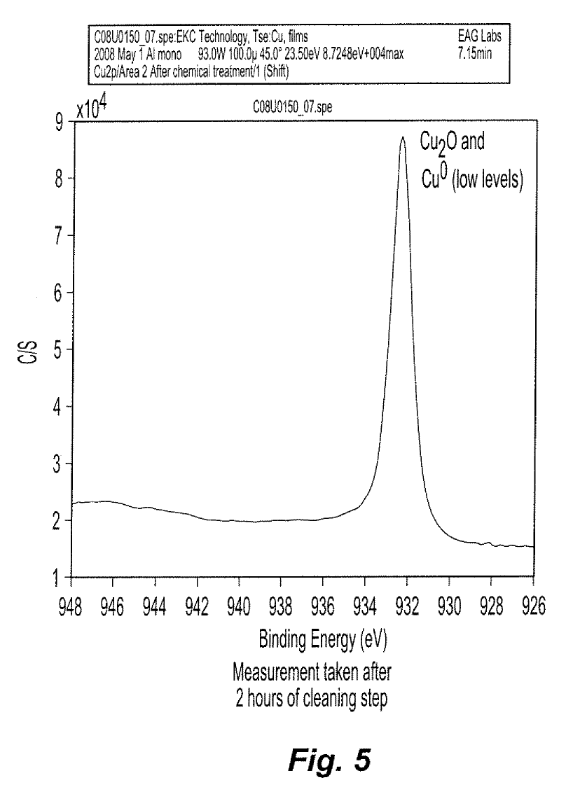

Even on the day the surface analysis was performed, there was a gap of two hours due to transportation to Evan Laboratory. Therefore, there is two hours as “queue time” for standard wafer fab processes for re-growth of copper oxide. FIG. 4 shows the result of ESCA analysis of the copper surface without any treatment, indicating a high concentration of Cu(II) oxide. FIG. 5 shows the efficacy Cu(II) oxide removal by the amidoxime solution.

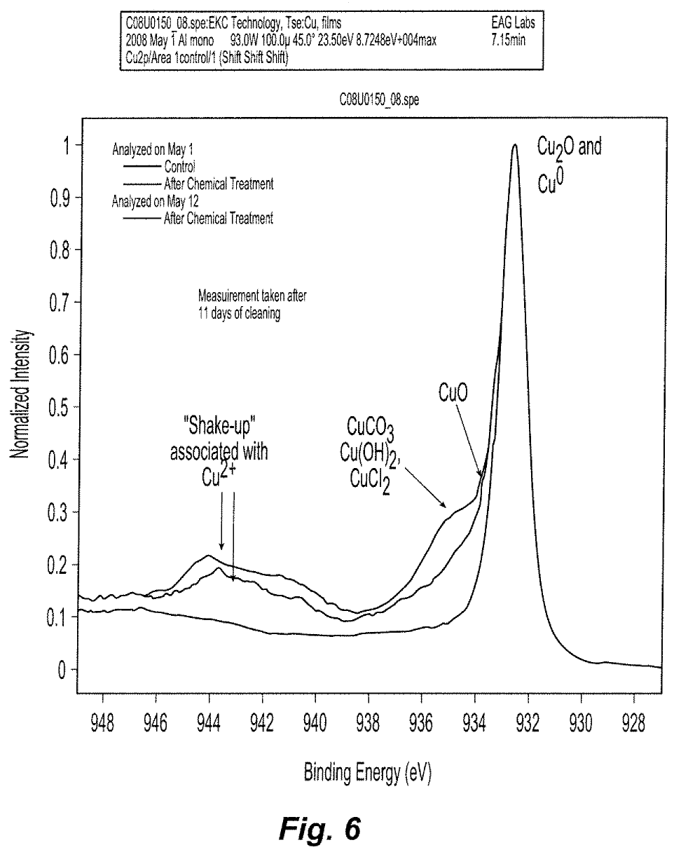

FIG. 6 shows that amidoxime also inhibits the growth of Cu(II) oxide.

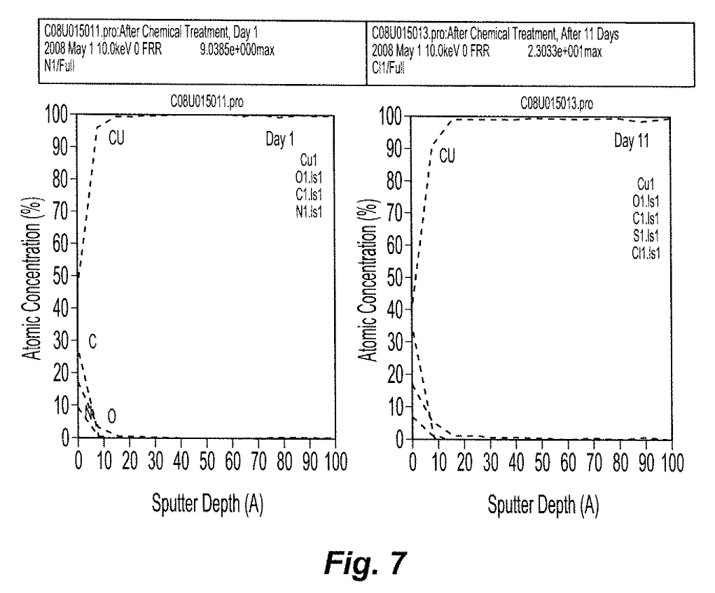

FIG. 7 is the Auger depth profile analysis of the cleaning treated copper surface. The result suggests that the Cu(I) and Cu(II) oxide thickness have not increased.

### Example 62

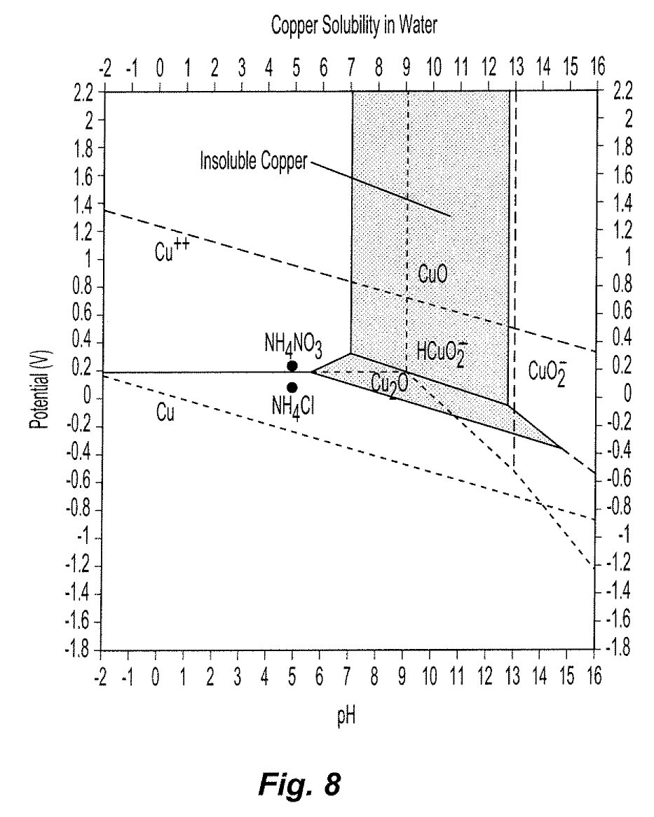

To the contrary of solubility of Cu(II) oxide, as shown in this Pourbaix diagram, Cu—H2O system forms insoluble oxides and hydroxides at pH of 7-12. (Ref: M. J. N. Pourbaix, Atlas of Chemical Equilibria in Aqueous Solutions, National Assoc. of Corrosion Enginneers, Houston, Tex., 1974.), amidoxime remove Cu(II) oxide effectively at pH of 9-11. FIG. 8 shows the Copper Pourbaix diagram.

### Example 63

Another embodiment of the present invention is a stripping and cleaning agent for removing dry-etching photoresist residues, and a method for forming an aluminum based line pattern using the stripping and cleaning agent. The stripping and cleaning agent contains (a) from 5 to 50% by weight of one or more compounds with one or more chelating groups/agents, at least one being an amidoxime functional group/compound; (h) from 0.5 to 15% by weight of a fluorine compound; and (c) a solvent, including water The inventive method is advantageously applied to treating a dry-etched semiconductor substrate with the stripping and cleaning agent. The semiconductor substrate comprises a semiconductor wafer having thereon a conductive layer containing aluminum. The conductive layer is dry-etched through a patterned photoresist mask to form a wiring body having etched side walls. The dry etching forms a side wall protection film on the side walls. In accordance with the inventive method, the side wall protection film and other resist residues are completely released without corroding the wiring body. See, U.S. Pat. No. 5,630,904.

### Example 64

U.S. Pat. No. 6,927,176 describes the effectiveness of chelating compounds due to their binding sites as illustrated in FIGS. 2a and 2b. It highlights that there are 6 binding sites

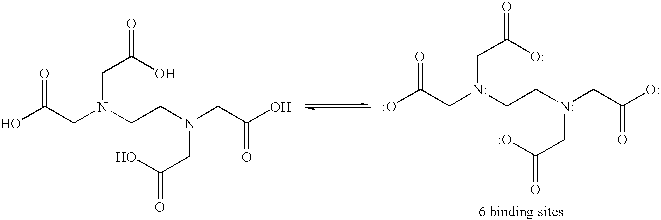

By the same principal applying to a amidoxime from the conversion of a cyanoethylation compound of ethylenediamine, it results a total of 14 binding sites, as depicted in the following

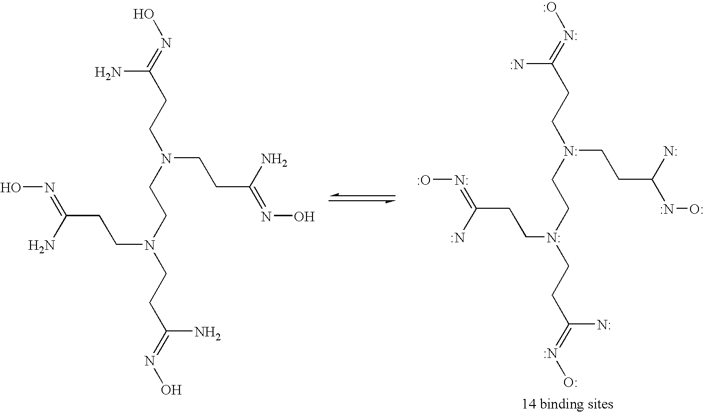

### (1,2,3,4,5,6-(hexa-(2-amidoximo)ethoxy)hexane Hexitol

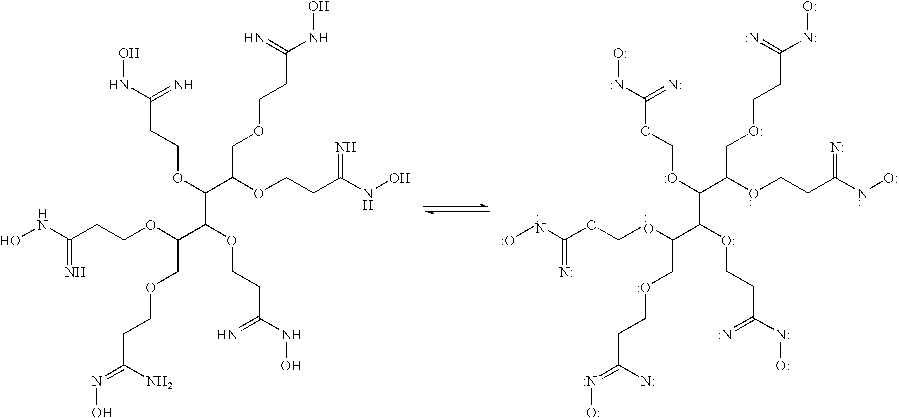

has a total of 18 binding sites. The claimed amidoxime chelating agent can substitute in this application to replace polyacrylates, carbonates, phosphonates, and gluconates, ethylenediaminetetraacetic acid (EDTA), N,N′-bis(2-hydroxyphenyl)ethylenediiminodiacetic acid (HPED), triethylenetetranitrilohexaacetic acid (TTHA), desferriferrioxamin B, N,N′,N″-tris[2-(N-hydroxycarbonyl)ethyl]-1,3,5-benzenetricarboxamide (BAMTPH), and ethylenediaminediorthohydroxyphenylacetic acid (EDDHA).

Cleaning solutions of the present application include compositions comprising:

A) An Organic Compound with One or More Amidoxime Functional Group.

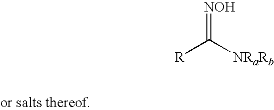

Firstly considering the amidoxime functional group itself, in one embodiment, Rₐ and R_{b} are independently hydrogen, alkyl, hetero-alkyl, alkyl-aryl, or alkyl-heteroaryl groups. R is independently selected from alkyl, alkyl-aryl, or alkyl-heteroaryl groups. In these two embodiments, chelation of the amidoxime to metal centres may be favoured because, in reaction with a metal centre, a proton can be lost from NRₐR_{b} so as to form a nominally covalent bond with the metal centre.

In another embodiment, NRₐR_{b} is further substituted with R_{c} so the amidoxime has the following chemical formula:

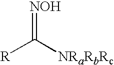

In this case, a counter-ion balances the positive charge on the nitrogen atom. Any counter-ion may be used, for example chloride, bromide, iodide, a SO₄ ion, a PF₆ ion or a ClO₄ ion. R_{c} may be hydrogen or an R group as defined below.

It is noted that Rₐ, R_{b} and/or R_{c} can join onto one another and/or join onto R so as to form one or more cycles.

It is also noted that amidoxime can exist as their tautomers:

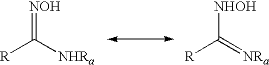

Compounds that exist mainly or wholly in this tautomeric form are included within the scope of the present invention.

Accordingly, the amidoxime functional group includes the following functionalities and their tautomers:

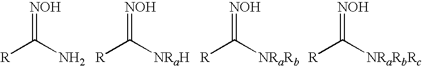

wherein R may be connected to one or more of Rₐ, R_{b} and R_{c}.

For example, the amidoxime functional group includes within its scope:

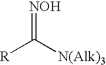

wherein Alk is an alkyl group as defined below. The three alkyl groups may be independently selected or may be the same. In one embodiment, the alkyl group is methyl or ethyl.

Turning now to the R group, R may be an alkyl group (in other words, a group containing carbon and hydrogen). The alkyl group may be completely saturated or may contain unsaturated groups (i.e. may contain alkene and alkyne functional groups, so the term “alkyl” encompasses the terms “alkylene” and “alkylyne” within its scope). The alkyl group may be straight-chained or branched.

The alkyl group may contain any number of carbon and hydrogen atoms. While alkyl groups having a lesser number of carbon atoms tend to be more soluble in polar solvents such as DMSO and water, alkyl groups having a greater number of carbons can have other advantageous properties, for example surfactant properties. Therefore, in one embodiment, the alkyl group contains 1 to 10 carbon atoms, for example the alkyl group is a lower alkyl group containing 1 to 6 carbon atoms. In another embodiment, the alkyl group contains 10 or more carbon atoms, for example 10 to 24 carbon atoms.

The alkyl group may be unsubstituted (i.e. the alkyl group contains only carbon and hydrogen). The unsubstituted alkyl group may be unsaturated or saturated. Examples of possible saturated unsubstituted alkyl groups include methyl, ethyl, n-propyl, sec-propyl, cyclopropyl, n-butyl, sec-butyl, tert-butyl, cyclobutyl, pentyl (branched or unbranched), hexyl (branched or unbranched), heptyl (branched or unbranched), octyl (branched or unbranched), nonyl (branched or unbranched), and decyl (branched or unbranched). Saturated unsubstituted alkyl groups having a greater number of carbons may also be used. Cyclic alkyl groups may also be used, so the alkyl group may comprise, for example, a cyclopropyl group, a cylcobutyl group, a cyclopentyl group, a cyclohexyl group, a cycloheptyl group, a cyclooctyl group, a cylcononyl group and/or a cyclodecyl group. These cyclic alkyl groups may directly append the amidoxime group or may be joined to the amidoxime through one or more carbon atoms.

Examples of amidoxime compounds containing unsubstituted saturated alkyl groups include:

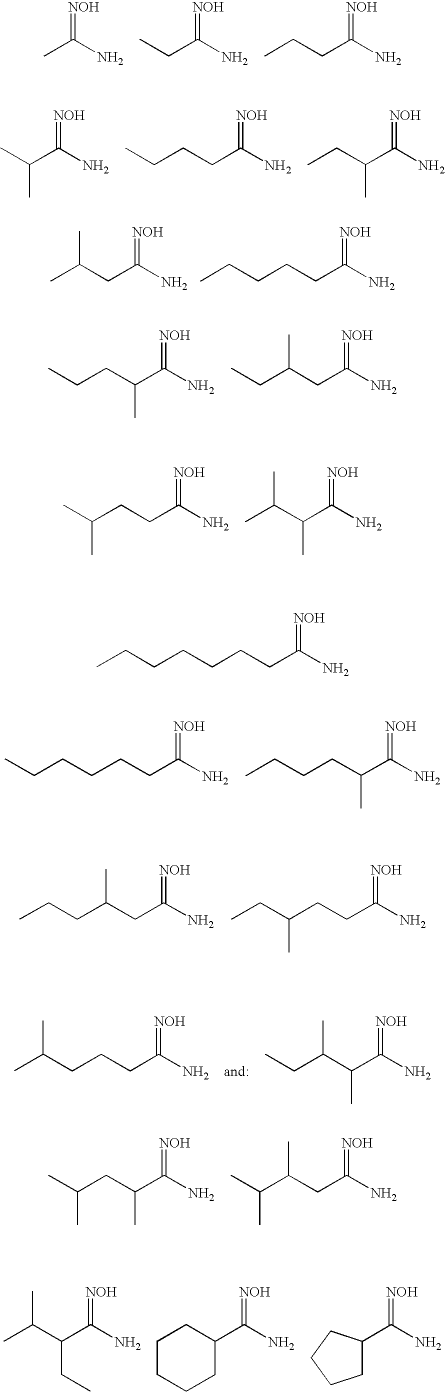

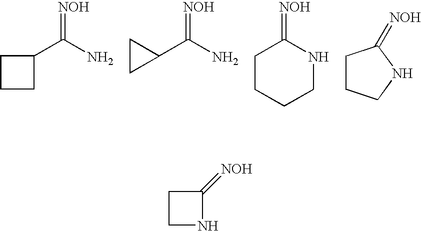

Examples further include:

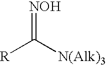

wherein Alk is methyl or ethyl and R is an alkyl group, typically but not necessarily straight chained. R may be for example an alkyl group containing 8 to 25 carbon atoms. If the alkyl group is substituted, it may for example be substituted at the opposite end of the alkyl group to the amidoxime group. For example, it may be substituted antipodally to the amidoxime group by one or more halogens, for example fluorine.

Examples further include alkyl groups appending two or more amidoxime functional groups.

For example, the amidoxime may be:

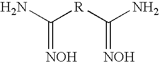

where R is an alkyl group. For example, R may be a straight chained alkyl group, such as an unsubstituted straight chained alkyl group. Examples of suitable groups include methyl, ethyl, propyl, butyl, pentyl, hexyl, heptyl, octyl, nonyl and decyl.

Specific examples of unsubstituted saturated alkyl amidoximes are:

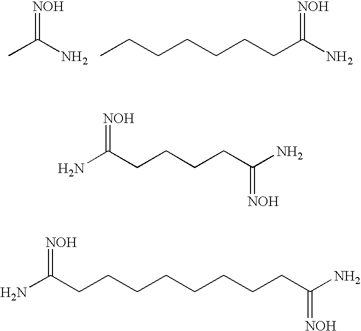

If the alkyl group is unsaturated, it may be any of the groups just listed except for having one or more unsaturated carbon-carbon bonds (so it may contain one or more alkene and/or alkyne groups). These unsaturated group(s) may optionally be in conjugation with the amidoxime group. A specific example of an unsubstituted unsaturated alkyl amidoxime molecules is:

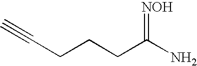

The alkyl group may also be substituted with one or more hetero-atoms or group of hetero-atoms. If more than one hetero-substituent is present, the substituents are independently selected from one another unless they form a part of a particular functional group (e.g. an amide group). (Groups containing hetero-atoms joined to carbon atoms are contained within the scope of the term “heteroalklyl” as discussed below). One or more of the substituents may be a halogen atom, including fluorine, chlorine, bromine or iodine, —OH, ═O, —NH₂, ═NH, —NHOH, ═NOH, OPO(OH)₂, —SH, ═S or —SO₂OH. In one embodiment, the substituent is an oxime group (═NOH). The alkyl group may also be itself substituted with one or more amidoxime functional groups.

If the alkyl group is substituted with ═O, the alkyl group may comprise an aldehyde, a ketone, a carboxylic acid or an amide. Preferably, there is an enolizable hydrogen adjacent to the ═O, ═NH or ═NOH (i.e. there is a hydrogen in the alpha position to the carbonyl). The alkyl group may comprise the following functionality: —(CZ₁)-CH—(CZ₂)-, wherein Z₁ and Z₂ are independently selected from O, NH and NOH. The CH in this group is further substituted with hydrogen or an alkyl group or joined to the amidoxime functional group.

Thus, an alkyl group appending an amidoxime group may simply be substituted with, for example one or more independently-selected halogens, for example fluorine, chlorine, bromine or iodine. In one embodiment, the halogens are substituted at the antipodal (i.e. opposite) end of the alkyl group to the amidoxime group. This can for example provide surfactant activity, in particular for example if the halogen is fluorine.

A specific example of an amidoxime group substituted with a substituted alkyl group is:

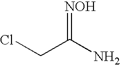

Details of the characterization of this molecule are given in the examples.

Compounds that are substituted in a β position are conveniently synthesized from readily-available starting materials.

Examples of such compounds include.

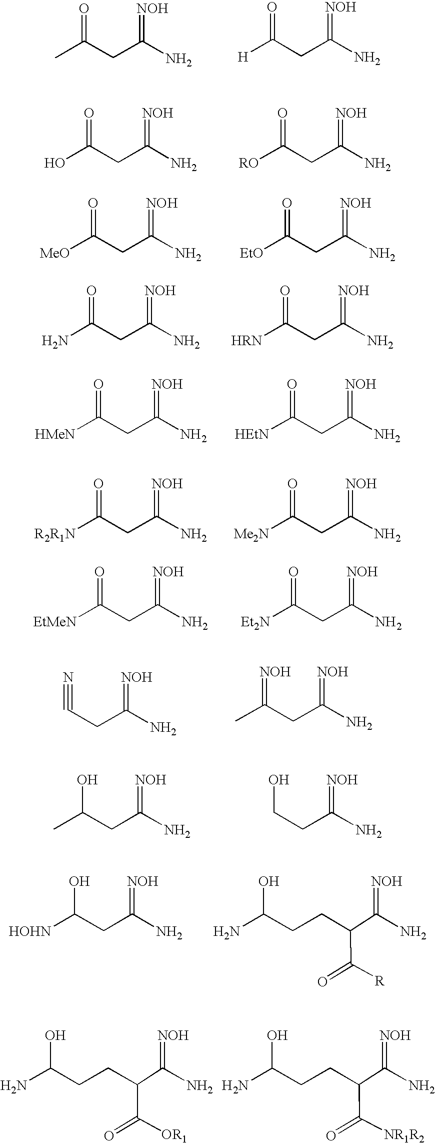

wherein R₁ and R₂ are independently-selected alkyl groups or hydrogen atoms.

Specific examples of substituted alkyl amidoxime molecules are:

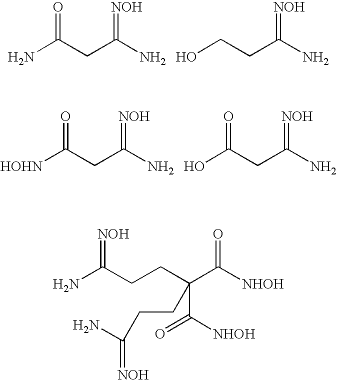

It should be noted that some of these molecules can exist as different isomers. For example:

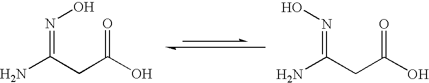

The different isomers can be differentiated by carbon-13 NMR. Characterization of this isomer is provided in the example.

R may be a heteroalkyl group. The term heteroalkyl refers to optionally a first alkyl group connected to one or more independently-selected hetero-atoms or groups of hetero-atoms, which itself is substituted with one or more independently-selected groups containing one or more carbon atoms. The presence of the first alkyl group is optional because the amidoxime group may be attached directly to the one or more heteroatoms. As an illustrative example, an alkyl group substituted with an ether group is a heteroalkyl group because the alkyl group is substituted with oxygen, which itself is substituted with a second alkyl group. Alternatively, an —O—CH₃ group is an example of a heteroalkyl group.

When R is a heteroalkyl group, the amidoxime may have the following chemical structure:

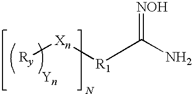

where “n” varies from 1 to N and y varies from 1 to Yₙ; N varies from 0 to 3; Yₙ varies from 0 to 5. In this formula, R₁ is independently-selected alkylene groups; R_{y} is independently selected from alkyl, or hetero-alkyl groups, or adjoins R₁ so to form a heterocycle with the directly appending Xₙ. R₁ may also be a direct bond, so that the amidoxime group is connected directly to the one or more heteroatoms. Xₙ is a heteroatom or a group of heteroatoms selected from boron, nitrogen, oxygen, silicon, phosphorus and sulphur. Each heteroatom or group of heteroatoms and each alkyl group is independently selected from one another. The above formula includes an amidoxime group directly bearing an alkyl group. The alkyl group is substituted with N independently-selected heteroatoms or groups of heteroatoms. Each heteroatom or group of heteroatoms is itself substituted with one or more independently-selected alkyl groups or hetero-alkyl groups.

X is one or more hetero-atoms. For example, X may be or may comprise boron, nitrogen, oxygen, silicon, phosphorus or sulphur. In one embodiment, X is oxygen. In this case, X may be part of an ether group (—O—), an ester (—O—CO—), —O—CO—O—, —O—CO—NH—, —O—CO—NR₂—, —O—CNH—, —O—CNH—O—, —O—CNH—NH—, —O—CNH—N₂—, —O—CNOH—, —O—CNOH—O—, —O—CNOH—NH— or —O—CNOH—NR₂—, wherein R₂ is independently selected alkyl group, hetero-alkyl group, or hetero-aryl group. In another embodiment, X is a nitrogen atom. In this case, X may be part of one of the following groups: —NR₂H, —NR₂—, —NR₂R₃— (with an appropriate counter-ion), —NHNH—, —NH—CO—, —NR2-CO—, —NH—CO—O—, —NH—CO—NH—, —NH—CO—NR₂—, —NR₂—CO—NH—, —NR₂—CO—NR₃—, —NH—CNH—, —NR₂—CNH—, —NH—CNH—O—, —NH—CNH—NH—, —NH—CNH—NR₂—, —NR₂—CNH—NH—, —NR₂—CNH—NR₃—, —NH—CNOH—, —NR2-CNOH—, —NH—CNOH—O—, —NH—CNOH—NH—, —NH—CNOH—NR₂—, —NR₂—CNOH—NH—, —NR₂—CNOH—NR₃—. R₂ to R₃ are independently selected alkyl groups, hetero-alkyl groups, or hetero-aryl groups, wherein the heteroalkyl group and hetero-aryl group may be unsubstituted or substituted with one or more heteroatoms or group of heteroatoms or itself be substituted with another heteroalkyl group. If more than one hetero-substituent is present, the substituents are independently selected from one another unless they form a part of a particular functional group (e.g., an amide group).

In another embodiment, X comprises boron. In this case, X may also comprise oxygen. In another embodiment, X comprises phosphorus. In this case, X may also comprise oxygen, for example in an —OPO(OH)(OR₂) group or an —OPO(OR₂)(OR₃) group. In another embodiment, X comprises sulphur, for example as a thiol ether or as a sulphone.

The term heteroalkyl also includes within its scope cyclic alkyl groups containing a heteroatom. If X is N or O, examples of such groups include a lactone, lactam or lactim. Further examples of heteroalkyl groups include azetidines, oxetane, thietane, dithietane, dihydrofuran, tetrahydrofuran, dihydrothiophene, tetrahydrothiophene, piperidine, pyrroline, pyrrolidine, tetrahydropyran, dihydropyran, thiane, piperazine, oxazine, dithiane, dioxane and morpholine. These cyclic groups may be directly joined to the amidoxime group or may be joined to the amidoxime group through an alkyl group.

The heteroalkyl group may be unsubstituted or substituted with one or more hetero-atoms or group of hetero-atoms or itself be substituted with another heteroalkyl group. If more than one hetero-substituent is present, the substituents are independently selected from one another unless they form a part of a particular functional group (e.g. an amide group). One or more of the substituents may be a halogen atom, including fluorine, chlorine, bromine or iodine, —OH, ═O, —NH₂, ═NH, —NHOH, ═NOH, —OPO(OH)₂, —SH, ═S or —SO₂OH. In one embodiment, the substituent is an oxime group (═NOH). The heteroalkyl group may also be itself substituted with one or more amidoxime functional groups.

If the heteroalkyl group is substituted with ═O, the heteroalkyl group may comprise an aldehyde, a ketone, a carboxylic acid or an amide. Preferably, there is an enolizable hydrogen adjacent to the ═O, ═NH or ═NOH (i.e. there is a hydrogen in the alpha position to the carbonyl). The heteroalkyl group may comprise the following functionality: —(CZ₁)-CH—(CZ₂)-, wherein Z₁ and Z₂ are independently selected from O, NH and NOH. The CH in this group is further substituted with hydrogen or an alkyl group or heteroalkyl group or joined to the amidoxime functional group.

Amines are particularly versatile functional groups for use in the present invention, in part because of their ease of preparation. For example, by using acrylonitrile as described later, a variety of functionalized amines can be synthesized.

Examples include:

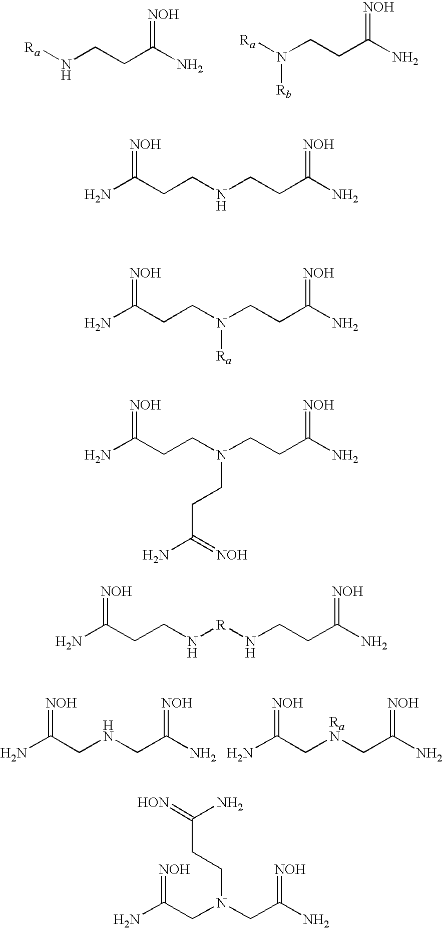

where Rₐ and R_{b} are independently-selected hydrogen, alkyl, hetero-alkyl, aryl, hetero-aryl, alkyl-aryl, or alkyl-heteroaryl groups.

R may itself be an alkylene croup or a heteroatom or group of heteroatoms. The heteroatoms may be unsubstituted or substituted with one or more alkyl groups. One or more of the hetero-atom substituents may be for example, a halogen atom, including fluorine, chlorine, bromine or iodine, —OH, ═O, —NH₂, ═NH, —NHOH, ═NOH, —OPO(OH)₂, —SH, ═S or —SO₂OH. In one embodiment, the substituent is an oxime group (═NOH).

R may be an aryl group. The term “aryl” refers to a group comprising an aromatic cycle. A particular example of an aryl substituent is a phenyl group.

The aryl group may be unsubstituted. A specific example of an amidoxime bearing an unsubstituted aryl is:

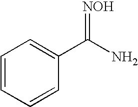

The aryl group may also be substituted with one or more alkyl groups, heteroalkyl groups or heteroatom substituents. If more than one substituent is present, the substituents are independently selected from one another.

Specific examples of amidoximes comprising a heteroalkyl group include:

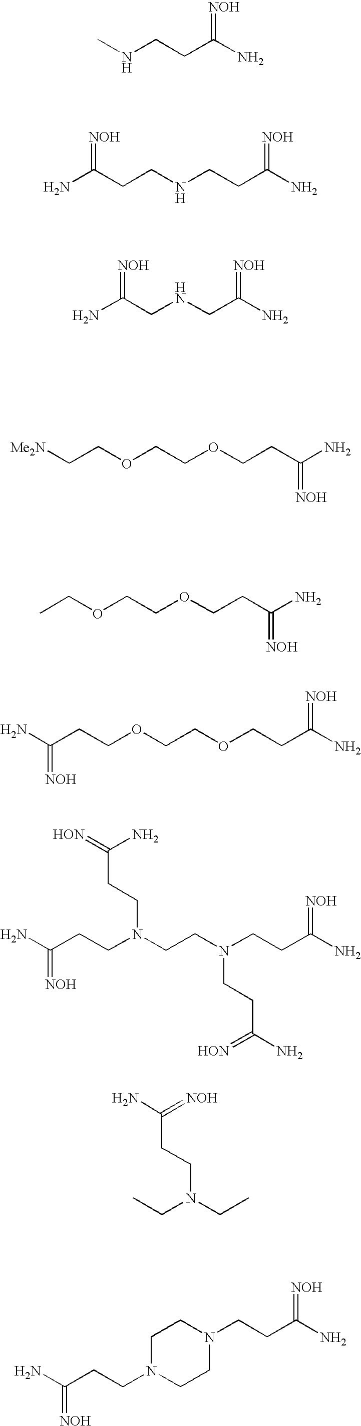

Specific examples of substituted aryl amidoxime molecules are:

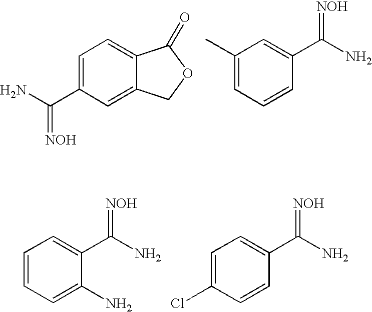

R may also be hetero-aryl. The term hetero-aryl refers to an aryl group containing one or more hetero-atoms in its aromatic cycle. The one or more hetero-atoms are independently-selected from, for example, boron, nitrogen, oxygen, silicon, phosphorus and sulfur. Examples of hetero-aryl groups include pyrrole, furan, thiophene, pyridine, melamine, pyran, thiine, diazine and thiazine.

The hetero-aryl group may be unsubstituted. A specific example of an unsubstituted heteroaryl amidoxime molecule is:

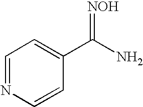

It should be noted that the heteroaryl group may be attached to the amidoxime group through its heteroatom, for example (the following molecule being accompanied by a counter anion):

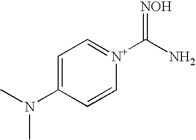

The hetero-aryl group may be substituted with one or more alkyl groups, heteroalkyl groups or hetero-atom substituents. If more than one substituent is present, the substituents are independently selected from one another.

One or more of the hetero-atom substituents may be, for example, a halogen atom, including fluorine, chlorine, bromine or iodine, —OH, ═O, —NH₂, ═NH, —NHOH, ═NOH, —OPO(OH)₂, —SH, ═S or —SO₂OH. The one or more alkyl groups are the alkyl groups defined previously and the one or more heteroalkyl groups are the heteroalkyl groups defined previously.

Within the scope of the term aryl are alkyl-aryl groups. The term “alkyl-aryl” refers to an amidoxime group bearing (i.e. directly joined to) an alkyl group. The alkyl group is then itself substituted with an aryl group. Correspondingly, within the scope of the term heteroaryl are alkyl-heteroaryl groups.

The alkyl group may be any alkyl group previously defined. The aryl/heteroaryl group may also be any aryl group previously defined.

Both the alkyl group and the aryl/heteroalkyl group may be unsubstituted. Specific examples of unsubstituted alkyl-aryl amidoxime molecules are:

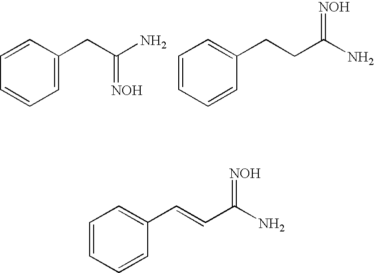

Alternatively, one or both of the alkyl group and the aryl/heteroalkyl group may be substituted. If the alkyl group is substituted, it may be substituted with one or more hetero-atoms or groups containing hetero-atoms. If the aryl/heteroalkyl group is substituted, it may be substituted with one or more alkyl groups, heteroalkyl groups or hetero-atom substituents. If more than one substituent is present, the substituents are independently selected from one another.

One or more of the hetero-atom substituents may be, for example, a halogen atom, including fluorine, chlorine, bromine or iodine, —OH, ═O, —NH₂, ═NH, —NHOH, ═NOH, —OPO(OH)₂, —SH, ═S or —SO₂OH. In one embodiment, the substituent is an oxime group (═NOH). The alkyl group may also be itself substituted with one or more amidoxime functional groups.

If the alkyl group is substituted with ═O, the alkyl group may comprise an aldehyde, a ketone, a carboxylic acid or an amide. Preferably, there is an enolizable hydrogen adjacent to the ═O, ═NH or ═NOH (i.e. there is a hydrogen in the alpha position to the carbonyl). The alkyl group may comprise the following functionality: —(CZ₁)-CH—(CZ₂)-, wherein Z₁ and Z₂ are independently selected from O, NH and NOH. The CH in this group is further substituted with hydrogen or an alkyl group or heteroalkyl group or joined to the amidoxime functional group.

Within the scope of the term aryl are also heteroalkyl-aryl groups. The term “heteroalkyl-aryl” refers to an amidoxime group bearing (i.e. directly joined to) an heteroalkyl group. The heteroalkyl group is then itself substituted with an aryl group. Correspondingly, within the scope of the term heteroaryl are also heteroalkyl-aryl groups.

The heteroalkyl group may be any alkyl group previously defined. The aryl/heteroaryl group may also be any aryl group previously defined.

Both the heteroalkyl group and the aryl/heteroaryl group may be unsubstituted. Alternatively, one or both of the heteroalkyl group and the aryl/heteroaryl group may be substituted. If the heteroalkyl group is substituted, it may be substituted with one or more hetero-atoms or groups containing hetero-atoms. If the aryl/heteroaryl group is substituted, it may be substituted with one or more alkyl groups, heteroalkyl groups or hetero-atom substituents. If more than one substituent is present, the substituents are independently selected from one another.

One or more of the hetero-atom substituents may be, for example, a halogen atom, including fluorine, chlorine, bromine or iodine, —OH, ═O, —NH₂, ═NH, —NHOH, ═NOH, —OPO(OH)₂, —SH, ═S or —SO₂OH. In one embodiment, the substituent is an oxime group (═NOH). The alkyl group may also be itself substituted with one or more amidoxime functional groups.

If the heteroalkyl group is substituted with ═O, the heteroalkyl group may comprise an aldehyde, a ketone, a carboxylic acid or an amide. Preferably, there is an enolizable hydrogen adjacent to the ═O, ═NH or ═NOH (i.e. there is a hydrogen in the alpha position to the carbonyl). The heteroalkyl group may comprise the following functionality: —(CZ₁)-CH—(CZ₂)-, wherein Z₁ and Z₂ are independently selected from O, NH and NOH. The CH in this group is further substituted with hydrogen or an alkyl group or heteroalkyl group or joined to the amidoxime functional group.

A preferred substituent to any type of R group is a tetra-valent nitrogen. In other words, any of the above groups may be substituted with —NRₐR_{b}R_{c} where Rₐ to R_{c} are independently-selected hydrogen, alkyl, hetero-alkyl, alkyl-aryl, or alkyl-heteroaryl groups. In one embodiment, Rₐ to R_{c} are unsubstituted saturated alkyl groups having 1 to 6 carbon atoms. For example, one or more of (for example all of) Rₐ to R_{c} are methyl and/or ethyl. With this substituent, the tetra-valent nitrogen is preferably substituted in an antipodal position to the amidoxime group. For example, if R is a straight-chained unsubstituted saturated alkyl group of the form (CH₂)ₙ, then the tetra-valent nitrogen is at one end of the alkyl group and the amidoxime group is at the other end. In this embodiment, n is preferably 1, 2, 3, 4, 5 or 6.

In one embodiment, the present invention provides an amidoxime molecule that contains only one amidoxime functional group. In another embodiment, the present invention provides an amidoxime molecule containing two or more amidoxime functional groups. In fact, a large number of functional groups can be contained in a single molecule, for example if a polymer has repeating units having appending amidoxime functional groups. Examples of amidoxime compounds that contain more than one amidoxime functional groups have been described previously throughout the specification.

Amidoximes may be conveniently prepared from nitrile-containing molecules as follows;

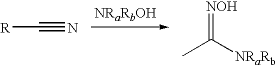

Typically, to prepare a molecule having Rₐ═R_{b}═H, hydroxylamine is used. If one or both of Rₐ and R_{b} in the desired amidoxime is not hydrogen, the amidoxime can be prepared either using the corresponding hydroxylamine or by further reacting the amidoxime once it has been formed. This may, for example, occur by intra-molecular reaction of the amidoxime.

Accordingly, amidoxime molecules containing more than one amidoxime functional groups can be conveniently prepared from precursors having more than one nitrile group. Specific amidoxime molecules having two amidoxime functional groups which have been synthesized in this way include:

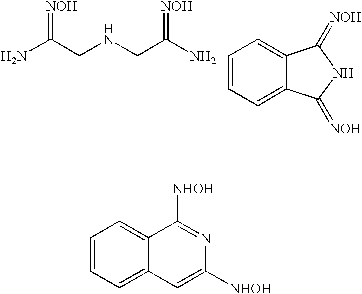

One preferred method of forming the nitrile precursors to the amidoximes of the present invention is by nucleophilic substitution of a leaving group with a nucleophile. Nucleophiles are well known to the person skilled in the art, see for example the Guidebook to Mechanism in Organic Chemistry by Peter Sykes. Examples of suitable nucleophiles are molecules having an OH, SH, NH— or a suitable CH— group, for example one having a low pKₐ (for example below about 15). For OH, SH and NH—, the hydrogen is optionally removed before acting as a nucleophile in order to augment its nucleophilicity. For CH—, they hydrogen is usually removed with a suitable base so that it can act as a nucleophile.

Leaving groups are well known to the person skilled in the art, see for example the Guidebook to Mechanism in Organic Chemistry by Peter Sykes. Examples of suitable leaving groups include Cl, Br, I, O-tosyl, O-mesolate and other leaving group well known to the person skilled in the art. The ability to act as a leaving group may be enhanced by adding an acid, either protic or Lewis.

For example, a nitrile can be formed accordingly:

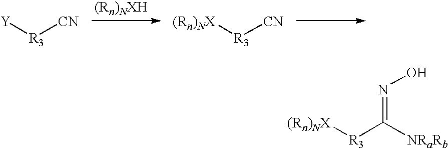

In this example, R₃ is independently selected from alkylene, heteroalkylene, arylene, heteroarylene, alkylene-heteroaryl, or alkylene-aryl group. Rₙ is independently selected from hydrogen, alkyl, heteroalkyl, aryl, heteroaryl, alkyl-heteroaryl, or alkyl-aryl group. X may be any a nucleophile selected from O, S, N, and suitable C. N varies from 1 to 3. Y is a leaving group.

For XH═OH, the OH may be an alcohol group or may, for example, be part of a hemiacetal or carboxylic acid group.

For X═NH—, the NH may be part of a primary or secondary amine (i.e. NH₂ or NHR₅), NH—CO—, NH—CNH—, N—CHOH— or —NHNR₅R₆ (wherein R₅ and R₆ are independently-selected alkyl, heteroalkyl, aryl, heteroaryl or alkyl-aryl).

For X═CH—, wherein a stabilized anion may be formed. XH may be selected from but not limited to —CHCO—R₅, —CHCOOH, —CHCN, —CHCO—OR₅, —CHCO—NR₅R₆, —CHCNH—R₅, —CHCNH—OR₅, —CHCNH—NR₅R₆, —CHCNOH—R₅, —CHCNOH—OR₅ and —CHCNOH—NR₅R₆.

A preferred example is:

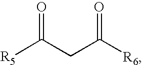

for example

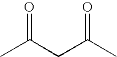

wherein R₅ and R₆ are independently-selected alkyl, heteroalkyl, aryl, heteroaryl or alkyl-aryl or a heteroatom optionally substituted with any of these groups. In one embodiment, either one or both of R₅ and R₆ are oxygen or nitrogen atoms optionally independently substituted with alkyl, heteroalkyl, aryl, heteroaryl or alkyl-aryl groups, for example:

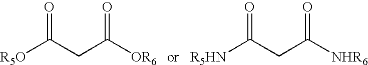

The compounds may also be formed by any type of nucleophilic reaction using any of the above nucleophiles.

The inventors have found one reaction in particular to be particularly versatile for producing nitrile precursors for amidoxime compounds:

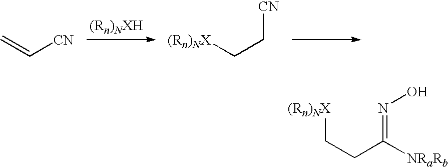

In this example, R₃ is independently selected from alkylene, heteroalkylene, arylene, heteroarylene, alkylene-heteroaryl, or alkylene-aryl group. Rₙ is independently selected from hydrogen, alkyl, heteroalkyl, aryl, heteroaryl, alkyl-heteroaryl, or alkyl-aryl group. X may be any a nucleophile selected from O, S, N, and suitable C. N varies from 1 to 3. Y is a leaving group.

For example, the acrylonitrile may have the following formula:

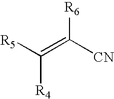

wherein R₄, R₅ and R₆ are independently selected from hydrogen, heteroatoms, heterogroups, alkyl, heteroalkyl, aryl and heteroaryl.

Accordingly, the present invention also relates to amidoxime compounds for use in semiconductor processing prepared by the addition of a nucleophile to an unsubstituted or substituted acrylonitrile. Once nucleophilic addition to the acrylonitrile has occurred, the intermediate can be functionalized using standard chemistry known to the person skilled in the art:

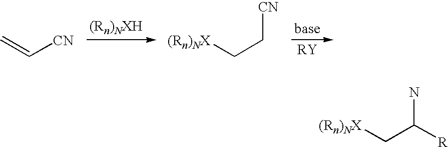

where Y is a leaving group as previously defined.

Examples of simple nucleophiles with show the adaptability of this reaction include:

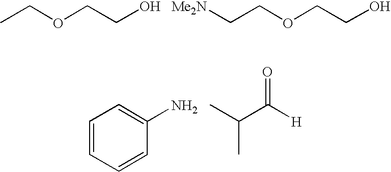

This reaction is particularly versatile, especially when applied to the synthesis of muitidentate amidoxime compounds (i.e. molecules containing two or more amidoxime functional groups). For example, it can be used to functionalize compounds having two or more NH groups. In one example, the reaction can be used to functionalize a molecule containing two or more primary amines.

For example:

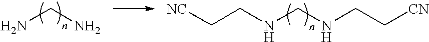

where n is 1 or more, for example 1 to 24.

Further functionalization of a primary amine is possible.

For example, a tetradentate amidoxime, for example the functional equivalent of EDTA, may be conveniently formed:

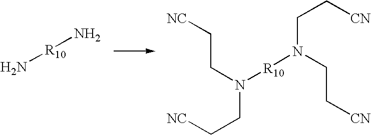

wherein R₁₀ is alkyl, heteroalkyl, aryl or heteroaryl. In an alternative conceived embodiment, R₁₀ is nothing: the starting material is hydrazine. An example of this reaction where R₁₀ is CH₂CH₂ is provided in the examples.

In a related embodiment, a molecule having two or more secondary amines can be functionalized:

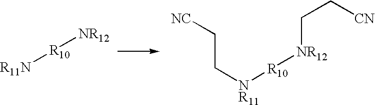

where R₁₀ is defined as above and R₁₁ and R₁₂ are independently selected alkyl, heteroalkyl, aryl or heteroaryl. Again, an embodiment where R₁₀ is nothing is contemplated.

For example, the secondary amines can be part of a cyclic system:

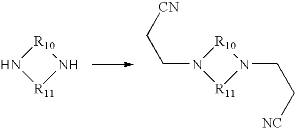

where R₁₀ and R₁₁ are defined above. For example, common solvent used in semiconductor processing can be functionalized with amidoxime functional groups. For example:

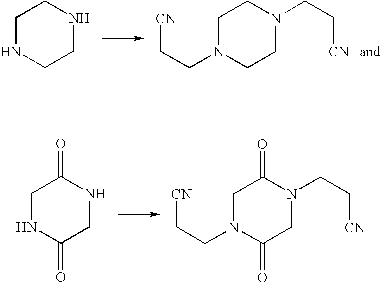

Details of theses reactions are contained in the examples.

Similarly, an oxygen nucleophile may be used to provide nitrile precursors to amidoxime molecules. In one embodiment, the nucleophile is an alcohol:

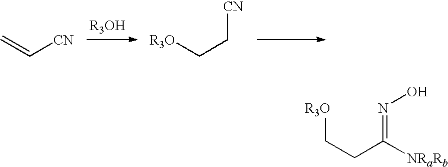

where R₃ is alkyl, heteroalkyl, aryl or heteroaryl.

For example, polyalcohol compounds may be functionalized. Poly-alcohols are molecules that contain more than one alcohol functional group. As an example, the following is a polyalcohol:

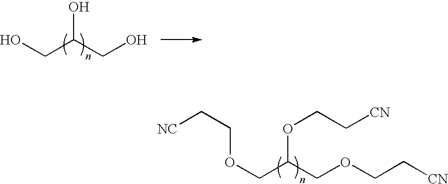

wherein n is 0 or more, for example 0 to 24. In one example, n is 0 (glycol). In another example, n is 6 (sorbitol).

In another example, the polyalcohol forms part of a polymer. For example, reaction may be carried out with a polymer comprising polyethylene oxide. For example, the polymer may contain just ethylene oxide units, or may comprise polyethylene oxide units as a copolymer (i.e. with one or more other monomer units). For example, the polymer may be a block copolymer comprising polyethylene oxide. For copolymers, especially block copolymers, the polymer may comprise a monomer unit not containing alcohol units. For example, the polymer may comprise blocks of polyethylene glycol (PEG). Copolymer (e.g. block copolymers) of polyethylene oxide and polyethylene glycol may be advantageous because the surfactant properties of the blocks of polyethylene glycol can be used and controlled.

Carbon nucleophiles can also be used. Many carbon nucleophiles are known in the art. For example, an enol group can act as a nucleophile. Harder carbon-based nucleophiles can be generated by deprotonation of a carbon. While many carbons bearing a proton can be deprotonated if a strong enough base is provided, it is often more convenient to be able to use a weak base to generate a carbon nucleophile, for example NaOEt or LDA. As a result, in one embodiment, a CH group having a pKₐ of 20 or less, for example 15 or less, is deprotonated to form the carbon-based nucleophile.

An example of a suitable carbon-based nucleophile is a molecule having the beta-diketone functionality (it being understood that the term beta-diketone also covers aldehydes, esters, amides and other C═O containing functional groups. Furthermore, one or both of the C═O groups may be replaced by NH or NOH).

For example:

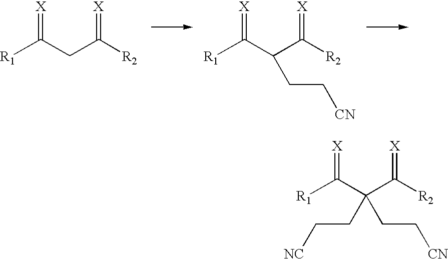

where R₁ and R₂ are independently selected alkyl groups, heteroalkyl groups, aryl groups, heteroaryl groups and heteroatoms.

A specific example of this reaction sequence where R₁═R₂=OEt is given in the examples.

Nitrile groups themselves act to lower the pKₐ of hydrogens in the alpha position. This in fact means that sometimes control of reaction conditions is preferably used to prevent a cyano compound, once formed by reaction of a nucleophile with acrylonitrile, from deprotonating at its alpha position and reacting with a second acrylonitrile group. For example, selection of base and reaction conditions (e.g. temperature) can be used to prevent this secondary reaction. However, this observation can be taken advantage of to functionalize molecules that already contain one or more nitrile functionalities. For example, the following reaction occurs in basic conditions:

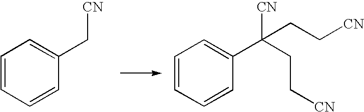

The cyanoethylation process usually requires a strong base as a catalyst. Most often such bases are alkali metal hydroxides such as, e.g., sodium oxide, lithium hydroxide, sodium hydroxide and potassium hydroxide. These metals, in turn, can exist as impurities in the amidoxime compound solution. The existence of such metals in the amidoxime compound solution is not acceptable for use in electronic, and more specifically, semiconductor manufacturing processes and as stabilizer for hydroxylamine freebase and other radical sensitive reaction chemicals.

Preferred alkali bases are metal ion free organic ammonium hydroxide compound, such as tetramethylammonium hydroxide, trimethylbenzylammonium hydroxide and the like.

B) Water

Within the scope of this invention, water may be introduced into the composition essentially only in chemically and/or physically bound form or as a constituent of the raw materials or compounds.

C) Solvent—from about 1% to 99% by Weight.

The compositions of the present invention also include 0% to about 99% by weight and more typically about 1% to about 80% by weight of a water miscible organic solvent, where the solvent(s) is/are preferably chosen from the group of water miscible organic solvents.

Examples of water miscible organic solvents include, but are not limited to, dimethylacetamide (DMAC), N-methylpyrrolidinone (NMP), N-Ethyl pyrrolidone (NEP), N-Hydroxyethyl Pyrrolidone (HEP), N-Cyclohexyl Pyrrolidone (CHP) dimethylsulfoxide (DMSO), Sulfolane, dimethylformamide (DMF), N-methylformamide (NMF), formamide, Monoethanol amine (MEA), Diglycolamine, dimethyl-2-piperidone (DMPD), morpholine, N-morpholine-N-Oxide (NMNO), tetrahydrofurfuryl alcohol, cyclohexanol, cyclohexanone, polyethylene glycols and polypropylene glycols, glycerol, glycerol carbonate, triacetin, ethylene glycol, propylene glycol, propylene carbonate, hexylene glycol, ethanol and n-propanol and/or isopropanol, diglycol, propyl or butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl, ethyl or propyl ether, dipropylene glycol methyl or ethyl ether, methoxy, ethoxy or butoxy triglycol, 1-butoxyethoxy-2-propanol, 3-methyl-3-methoxybutanol, propylene glycol t-butyl ether, and other amides, alcohols or pyrrolidones, ketones, sulfoxides, or multifunctional compounds, such as hydroxyamides or aminoalcohols, and mixtures of these solvents thereof. The preferred solvents, when employed, are dimethyl acetamide and dimethyl-2-piperidone, dimethylsufoxide and N-methylpyrrolidinone, diglycolamine, and monoethanolamine.

D) Acids—from about 0.001% to 15% by Weight.

Possible acids are either inorganic acids or organic acids provided these are compatible with the other ingredients.

Inorganic acids include hydrochloric acid, hydrofluoric acid, sulfuric acid, phosphoric acid, phosphorous acid, hypophosphorous acid, phosphonic acid, nitric acid, and the like.

Organic acids include monomeric and/or polymeric organic acids from the groups of unbranched saturated or unsaturated monocarboxylic acids, of branched saturated or unsaturated monocarboxylic acids, of saturated and unsaturated dicarboxylic acids, of aromatic mono-, di- and tricarboxylic acids, of sugar acids, of hydroxy acids, of oxo acids, of amino acids and/or of polymeric carboxylic acids are preferred.

The group of unbranched saturated or unsaturated monocarboxylic acids includes the following: methanoic acid (formic acid), ethanoic acid (acetic acid), propanoic acid (propionic acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid, dodecanoic acid (lauric acid), tridecanoic acid, tetradecanoic acid (myristic acid), pentadecanoic acid, hexadecanoic acid (palmitic acid), heptadecanoic acid (margaric acid), octadecanoic acid (stearic acid), eicosanoic acid (arachidic acid), docosanoic acid (behenic acid), tetracosanoic acid (lignoceric acid), hexacosanoic acid (cerotic acid), triacontanoic acid (melissic acid), 9c-hexadecenoic acid (palmitoleic acid), 6c-octadecenoic acid (petroselic acid), 6t-octadecenoic acid (petroselaidic acid), 9c-octadecenoic acid (oleic acid), 9t-octadecenoic acid (elaidic acid), 9c,12c-octadecadienoic acid (linoleic acid), 9t,12t-octadecadienoic acid (linolaidic acid) and 9c,12c,15c-octadecatrienoic acid (linolenic acid).

The group of branched saturated or unsaturated monocarboxylic acids includes the following: 2-methylpentanoic acid, 2-ethylhexanoic acid, 2-propylheptanoic acid, 2-butyloctanoic acid, 2-pentylnonanoic acid, 2-hexyldecanoic acid, 2-heptylundecanoic acid, 2-octyldodecanoic acid, 2-nonyltridecanoic acid, 2-decyltetradecanoic acid, 2-undecylpentadecanoic acid, 2-dodecylhexadecanoic acid, 2-tridecylheptadecanoic acid, 2-tetradecyloctadecanoic acid, 2-pentadecylnonadecanoic acid, 2-hexadecyleicosanoic acid, 2-heptadecylheneicosanoic acid.

The group of unbranched saturated or unsaturated di- or tricarboxylic acids includes the following: propanedioic acid (malonic acid), butanedioic acid (succinic acid), pentanedioic acid (glutaric acid), hexanedioic acid (adipic acid), heptanedioic acid (pimelic acid), octanedioic acid (suberic acid), nonanedioic acid (azelaic acid), decanedioic acid (sebacic acid), 2c-butenedioic acid (maleic acid), 2t-butenedioic acid (fumaric acid), 2-butynedicarboxylic acid (acetylenedicarboxylic acid).

The group of aromatic mono-, di- and tricarboxylic acids includes the following: benzoic acid, 2-carboxybenzoic acid (phthalic acid), 3-carboxybenzoic acid (isophthalic acid), 4-carboxybenzoic acid (terephthalic acid), 3,4-dicarboxybenzoic acid (trimellitic acid), and 3,5-dicarboxybenzoic acid (trimesionic acid).

The group of sugar acids includes the following: galactonic acid, mannonic acid, fructonic acid, arabinonic acid, xylonic acid, ribonic acid, 2-deoxyribonic acid, alginic acid.

From the group of hydroxy acids: hydroxyphenylacetic acid (mandelic acid), 2-hydroxypropionic acid (lactic acid), hydroxysuccinic acid (malic acid), 2,3-dihydroxybutanedioic acid (tartaric acid), 2-hydroxy-1,2,3-propanetricarboxylic acid (citric acid), ascorbic acid, 2-hydroxybenzoic acid (salicylic acid), and 3,4,5-trihydroxybenzoic acid (gallic acid).

The group of oxo acids includes the following: 2-oxopropionic acid (pyruvic acid) and 4-oxopentanoic acid (levulinic acid).

The group of amino acids includes the following: alanine, valine, leucine, isoleucine, proline, tryptophan, phenylalanine, methionine glycine, serine, tyrosine, threonine, cysteine, asparagine, glutamine, aspartic acid, glutamic acid, lysine, arginine, and histidine.

E) Bases—from about 1% to 45% by Weight.

Possible bases are either inorganic bases or organic bases provided these are compatible with the other ingredients.

Inorganic bases include sodium hydroxide, lithium hydroxide, potassium hydroxide, ammonium hydroxide and the like.

Organic bases including organic amines, and quaternary alkylammonium hydroxide which may include, but are not limited to, tetramethylammonium hydroxide (TMAH), TMAH pentahydrate, benzyltetramethylammonium hydroxide (BTMAH), TBAH, choline, and Tris(2-hydroxyethyl)methylammonium hydroxide (TEMAH).

F) Activator—from about 0.001% to 25% by Weight

According to the present invention, the cleaning compositions comprise one or more substances from the group of activators, in particular from the groups of polyacylated alkylenediamines, in particular tetraacetylethylenediamine (TAED), N-acylimides, in particular N-nonanoylsuccinimide (NOSI), acylated phenolsulfonates, in particular n-nonanoyl- or isononanoyloxybenzenesulfonate (n- or iso-NOBS) and n-methylmorpholiniumacetonitrile, methylsulfate (MMA), and “nitrile quaternary” compound in amounts of from 0.1 to 20% by weight, preferably from 0.5 to 15% by weight and in particular from 1 to 10% by weight, in each case based on the total composition to enhance the oxidation/reduction performance of the cleaning solutions. The “nitrile quats”, cationic nitrites has the formula:

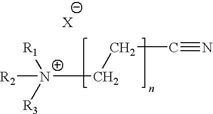

G) Compounds Having Oxidation and Reduction Potential—from about 0.001% to 25% by Weight.

These compounds include hydroxylamine and its salts, such as hydroxylamine chloride, hydroxylamine nitrate, hydroxylamine sulfate, hydroxylamine phosphate or its derivatives, such as N,N-diethylhydroxylamine, N-Phenylhydroxylamine.

Hydrazine and its derivatives; hydrogen peroxide; persulfate salts of ammonium, potassium and sodium, permanganate salt of potassium, sodium; and other sources of peroxide are selected from the group consisting of: perborate monohydrate, perborate tetrahydrate, percarbonate, salts thereof, and combinations thereof. For environmental reasons, hydroxylamine phosphate is not preferred.

Other compounds which may be used as ingredients within the scope of the present invention are the diacyl peroxides, such as, for example, dibenzoyl peroxide. Further typical organic compounds which have oxidation/reduction potentials are the peroxy acids, particular examples being the alkyl peroxy acids and the aryl peroxy acids. Preferred representatives are (a) peroxybenzoic acid and its ring substituted derivatives, such as alkylperoxybenzoic acids, but also peroxy-a-naphthoic acid and magnesium monoperphthalate, (b) the aliphatic or substituted aliphatic peroxy acids, such as peroxylauric acid, peroxystearic acid, c-phthalimidoperoxycaproic acid [phthaloiminoperoxyhexanoic acid (PAP)], o-carboxybenzamidoperoxycaproic acid, N-nonenylamidoperadipic acid and N-nonenylamidopersuccinate, and (c) aliphatic and araliphatic peroxydicarboxylic acids, such as 1,2-diperoxycarboxylic acid, 1,9-diperoxyazelaic acid, diperoxysebacic acid, diperoxybrassylic acid, the diperoxyphthalic acids, 2-decyldiperoxybutane-1,4-dioic acid, N,N-terephthaloyldi(6-aminopercaproic acid) may be used.

H) Other Chelating Agents—Preferably, the Cleaning Composition Comprises (by Weight of the Composition) from 0.0% to 15% of Additional One or More Chelant.

A further possible group of ingredients are the chelate complexing agents. Chelate complexing agents are substances which form cyclic compounds with metal ions, where a single ligand occupies more than one coordination site on a central atom, i.e. is at least “bidentate”. In this case, stretched compounds are thus normally closed by complex formation via an ion to give rings. The number of bonded ligands depends on the coordination number of the central ion.

Complexing groups (ligands) of customary complex forming polymers are iminodiacetic acid, hydroxyquinoline, thiourea, guanidine, dithiocarbamate, hydroxamic acid, amidoxime, aminophosphoric acid, (cycl.) polyamino, mercapto, 1,3-dicarbonyl and crown ether radicals, some of which have very specific activities toward ions of different metals.

For the purposes of the present invention, it is possible to use complexing agents of the prior art. These may belong to different chemical groups. Preferred chelating/complexing agents include the following, individually or in a mixture with one another.

1) polycarboxylic acids in which the sum of the carboxyl and optionally hydroxyl groups is at least 5, such as gluconic acid,

2) nitrogen-containing mono- or polycarboxylic acids, such as ethylenediaminetetraacetic acid (EDTA), N-hydroxyethylethylenediaminetriacetic acid, diethylenetraminepentaacetic acid, hydroxy-ethyliminodiacetic acid, nitridodiacetic acid-3-propionic acid, isoserinediacetic acid, N,N-di(.beta.-hydroxyethyl)glycine, N-(1,2-dicarboxy-2-hydroxyethyl)glycine, N-(1,2-dicarboxy-2-hydroxyethyl)-aspartic acid or nitrilotriacetic acid (NTA),

3) geminal diphosphonic acids, such as 1-hydroxyethane-1,1-diphosphonic acid (HEDP), higher homologs thereof having up to 8 carbon atoms, and hydroxy or amino group-containing derivatives thereof and 1-aminoethane-1,1-diphosphonic acid, higher homologs thereof having up to 8 carbon atoms, and hydroxy or amino group-containing derivatives thereof,

4) aminophosphonic acids, such as ethylenediamine-tetra(methylenephosphonic acid), diethylenetriaminepenta (methylenephosphonic acid) or nitrilotri(methylenephosphonic acid),

5) phosphonopolycarboxylic acids, such as 2-phosphonobutane-1,2,4-tricarboxylic acid, and

6) cyclodextrins.

Surfactants—from about 10 ppm to 5%.

The compositions according to the invention may thus also comprise anionic, cationic, and/or amphoteric surfactants as surfactant component.

Source of Fluoride Ions—from an Amount about 0.001% to 10%

Sources of fluoride ions include, but are not limited to, ammonium bifluoride, ammonium fluoride, hydrofluoric acid, sodium hexafluorosilicate, fluorosilicic acid and tetrafluoroboric acid.

The components of the claimed compositions can be metered and mixed in situ just prior dispensing to the substrate surface for treatment. Furthermore, analytical devices can be installed to monitor the composition and chemical ingredients can be re-constituted to mixture to the specification to deliver the cleaning performance. Critical parameters that can be monitored include, but are not limited to, physical and chemical properties of the composition, such as pH, water concentration, oxidation/reduction potential and solvent components.

The composition claims a range at point of use and also as mixtures which can be diluted to meet the specific cleaning requirements.

Summary of preferred amidoxime compounds from nitriles and not limited to

| | Nitrile (N) | Amidoxime (AO) |
|---|---|---|
| 3 | 3-hydroxypropionitrile | N′,3-dihydroxypropanimidamide |
| 4 | Acetonitrile | NN′-hydroxyacetimidamide |
| 5 | 3- | N′-hydroxy-3- |
| | methylaminopropionitrile | (methylamino)propanimidamide |
| 6 | Benzonitrile | N′-hydroxybenzimidamide |
| 8 | 3,3′ | 3,3′-azanediylbis(N′- |
| | iminodipropionitrile | hydroxypropanimidamide) |
| 9 | octanonitrile | N′-hydroxyoctanimidamide |
| 10 | 3-phenylpropionitrile | N′-hydroxy-3-phenylpropanimidamide |
| 11 | ethyl 2-cyanoacetate | 3-amino-N-hydroxy-3- |
| | | (hydroxyimino)propanamide |
| 12 | 2-cyanoacetic acid | 3-amino-3-(hydroxyimino)propanoic acid |
| 13 | 2-cyanoacetamide | 3-amino-3-(hydroxyimino)propanamide |
| 15 | adiponitrile | N′1,N′6-dihydroxyadipimidamide |
| 16 | sebaconitrile | N′1,N′10-dihydroxy-decanebis(imidamide) |
| 17 | 4-pyridinecarbonitrile | N′-hydroxyisonicotinimidamide |
| 18 | m-tolunitrile | N′-hydroxy-3-methylbenzimidamide |
| 19 | phthalonitrile | isoindoline-1,3-dione dioxime |
| 20 | glycolonitrile | N′,2-dihydroxyacetimidamide |
| 21 | chloroacetonitrile | 2-chloro-N′-hydroxyacetimidamide |
| 22 | benzyl cyanide | product N′-hydroxy-2- |
| | | phenylacetimidamide |
| 24 | Anthranilonitrile | 2-amino-N′-hydroxybenzimidamide |
| 25 | 3,3′ | 2,2′-azanediylbis(N′- |
| | iminodiacetonitrile | hydroxyacetimidamide) |
| 26 | 5-cyanophthalide | N′-hydroxy-1-oxo-1,3- |
| | | dihydroisobenzofuran-5-carboximidamide |
| 27 | 2- | 3-aminoisoquinolin-1(4H)-one oxime |
| | cyanophenylacetonitrile | or 3-(hydroxyamino)-3,4- |
| | | dihydroisoquinolin-1-amine |
| 29 | cinnamonitrile | N′-hydroxycinnamimidamide |
| 30 | 5-hexynenitrile | 4-cyano-N′-hydroxybutanimidamide |
| 31 | 4-chlorobenzonitrile | 4-chloro-N′-hydroxybenzimidamide |

For example, N3 represents 3-hydroxypropionitrile and AO3 is N′,3-dihydroxypropanimidamide from reacting 3-hydroxypropionitrile with hydroxylamine to form its corresponding amidoxime.

Summary of preferred amidoxime compounds from nitriles by cyanoethylation of nucleophilic compounds and not limited to the list below:

| | | Cyanoethylated | Amidoxime from |
|---|---|---|---|
| | Nucleophilic | Compounds | cyanoethylated |
| ID | compounds | (CE) | compounds (AO) |
| 01 | Sorbitol | 1,2,3,4,5,6-hexakis-O-(2- | 1,2,3,4,5,6-hexakis-O-[3-(hydroxyamino)- |
| | | kyanoetyl)hexitol | 3-iminopropyl Hexitol, |
| 07 | ethylenediamine | 3,3′,3″,3′″-(ethane-1,2- | 3,3′,3″,3″′-(ethane-1,2- |
| | | diylbis(azanetriyl)) | diylbis(azanetriyl))tetrakis |
| | | tetrapropanenitrile | (N′-hydroxypropanimidamide) |
| 28 | ethylene glycol | 3,3′-(ethane-1,2- | 3,3′-(ethane-1,2-diylbis(oxy))bis |
| | | diylbis(oxy))dipropanenitrile | (N′-hydroxypropanimidamide) |
| 34 | diethylamine | 3-(diethylamino)propanenitrile | 3-(diethylamino)-N′- |
| | | | hydroxypropanimidamide |
| 35 | piperazine | 3,3′-(piperazine-1,4- | 3,3′-(piperazine-1,4-diyl) |
| | | diyl)dipropanenitrile | bis(N′-hydroxypropanimidamide) |
| 36 | 2-ethoxyethanol | 3-(2-ethoxyethoxy) | 3-(2-ethoxyethoxy)-N′- |
| | | propanenitrile | hydroxypropanimidamide |
| 37 | 2-(2- | 3-(2-(2-(dimethylamino) | 3-(2-(2-(dimethylamino)ethoxy)ethoxy)- |
| | dimethylamino | ethoxy)ethoxy)propanenitrile | N′-hydroxypropanimidamide |
| | ethoxy)ethanol | | |
| 38 | isobutyraldehyde | 4,4-dimethyl-5-oxo | N′-hydroxy-4,4-dimethyl-5- |
| | | pentanenitrile | oxopentanimidamide |
| 39 | diethyl malonate | diethyl 2,2-bis(2-cyanoethyl) | 2,2-bis(3-amino-3- |
| | | malonate | (hydroxyimino)propyl)malonic acid |
| 40 | aniline | 3-(phenylamino) propanenitrile | N′-hydroxy-3-(phenylamino) |
| | | | propanimidamide |
| 41 | ammonia | 3,3′,3″-nitrilotri | 3,3′,3″-nitrilotris |
| | | propanenitrile | (N′-hydroxypropanimidamide) |
| 42 | diethyl malonate | 2,2-bis(2-cyanoethyl) malonic | 2,2-bis(3-amino-3- |
| | | acid | (hydroxyimino)propyl)malonic acid |
| 43 | Glycine(Mono | 2-(2-cyanoethylamino)acetic | 2-3(amino-3- |
| | cyanoethylated) | acid | (hydroxyimino)propylamino)acetic acid |
| 44 | Glycine | 2-(bis(2-cyanoethyl)amino) | 2-(bis(3-amino-3- |
| | (Dicyanothylated) | acetic acid | (hydroxyimino)propyl)amino)acetic acid |
| 45 | malononitrile | Propane-1,1,3-tricarbonitrile | N1,N′1,N′3-trihydroxypropane-1,1,3- |
| | | | tris(carboximidamide) |
| 46 | cyanoacetamide | 2,4-dicyano-2-(2- | 5-amino-2-(3-amino-3- |
| | | cyanoethyl)butanamide | (hydroxyimino)propyl)-2-(N′- |
| | | | hydroxycarbamimidoyl)-5- |
| | | | (hydroxyimino)pentanamide |
| 47 | Pentaerythritol | 3,3′-2,2-bis((2-cyanoethoxy) | 3,3′-(2,2-bis(3-(hydroxyamino)-3- |
| | | methyl) propane-1,3- | iminopropoxy)methyl)propane-1,3- |
| | | diyl)bis(oxy) dipropanenitrile | diyl)bis(oxy)bis(N- |
| | | | hydroxypropanimidamide) |
| 48 | N-methyl | 3,3′-(2,2′-(methylazanediyl) | 3,3′-(2,2′-(methylazanediyl) |
| | diethanol amine | bis(ethane-2,1-diyl) | bis(ethane-2,1-diyl)bis(oxy))bis |
| | | bis(oxy))dipropanenitrile | (N′-hydroxypropanimidamide) |
| 49 | glycine anhydride | 3,3′-(2,5-dioxopiperazine-1,4- | 3,3′-(2,5′-dioxopiperazine-1,4-diyl)bis(N′- |
| | | diyl)dipropanenitrile | hydroxyimino)propyl)acetamide |
| 50 | acetamide | N,N-bis(2-cyanoethyl)acetamide | N,N-bis(3-amino-3- |
| | | | (hydroxyimino)propyl)acetamide |
| 51 | anthranilonitrile | 3,3′-(2-cyanophenylazanediyl) | 3,3′-(2-(N′- |
| | | dipropanenitrile | hydroxycarbamimidoyl)phenylazanediyl)bis |
| | | | (N′-hydroxypropanimidamide) |
| 52 | diethanolamine | 3,3′-(2,2′-(2- | 3,3′-(2,2′-(3-amino-3- |
| | | cyanoethylazanediyl)bis(ethane- | (hydroxyimino)propylazanediyl) |
| | | 2,1-diyl)bis(oxy))dipropane | bis(ethane-2,1,diyl))bis(oxy)bis |
| | | nitrile | (N′-hydroxypropanimidamide |

For example, CE36 represents cyanoethylated product of ethylene glycol and AO36 is from reacting 3-(2-ethoxyethoxy) propanenitrile with hydroxylamine to form its corresponding amidoxime.

While the invention has been described and illustrated herein by references to various specific materials, procedures and examples, it is understood that the invention is not restricted to the particular combinations of materials and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

What is claimed is:

1. A method of cleaning a polishing pad surface subsequent to chemical-mechanical polishing (CMP) a wafer surface containing copper (Cu) or a Cu-based alloy, the method comprising applying to the polishing pad surface a cleaning composition comprising from about 2 ppm to about 50 percent by weight of one or more compounds having at least one amidoxime functional group in water, optionally, with an acid or a base in amount such that the composition effectively solubilizes the copper and copper alloy.

2. The method of claim 1, wherein the water is deionized water.

3. The method according to claim 1, comprising applying the composition to a rotating polishing pad at a flow rate of about 100 to about 600 ml/min.

4. The method according to claim 3, comprising applying the composition to the polishing pad for about 3 seconds to about 20 seconds after conducting CMP on each of a plurality to wafers having a surface comprising Cu or Cu alloy.

5. A method comprising the steps of:
(a) conducting chemical-mechanical polishing (CMP) on a first wafer surface of a first wafer containing copper (Cu) or a Cu-based alloy on a surface of a polishing pad;
(b) removing the first wafer from the pad;
(c) applying to the polishing pad surface a cleaning compositions wherein the cleaning composition is a solution comprising about 2 ppm to about 50 percent by weight of one or more compounds having at least one amidoxime functional group in water, optionally, with an acid or a base in amount such that the composition effectively solubilize the copper and copper alloy;
(d) rinsing the polishing pad surface with water to remove any cleaning composition on the polishing surface;
(e) conducting CMP on a second wafer; and then
(f) repeating steps (b) through (e) one or more times.

6. The method of claim 5 wherein the water is deionized water.

7. The method according to claim 5, comprising applying the solution to a rotating polishing pad at a flow rate of about 100 to about 600 ml/min.

8. The method according to claim 6, comprising applying the composition to the rotating polishing pad for about 3 seconds to about 20 seconds.

9. A method of cleaning a surface of a polishing pad, comprising:
(a) conducting chemical-mechanical polishing (CMP) on a first wafer on the surface of the polishing pad;
(b) removing the first wafer from the polishing pad;
(c) applying to the polishing pad surface a cleaning composition, wherein the cleaning composition is a solution comprising from about 2 ppm to about 50 percent by weight of one or more compounds having at least one amidoxime functional group in deionized water, optionally, with an acid or a base in amount such that the composition effectively solubilize the copper and copper alloy; and
(d) cleaning the polishing pad surface with the cleaning composition.

10. The method of claim 9, wherein the cleaning composition further comprises hydrogen peroxide or hydroxylamine, wherein the mixing ratio of the one or more compounds having at least one amidoxime functional group: hydrogen peroxide or hydroxylamine: water ranges from about 1:4:20 to about 1:1:5, wherein the waiting time for allowing the solution to react with the residue is between about 30 to about 180 seconds, and wherein the solution is applied to the polishing pad at a heated temperature between about 40° C. and about 80° C.

11. The method of claim 9 wherein the one or more compounds having at least one amidoxime functional group have at least one of the following structures: 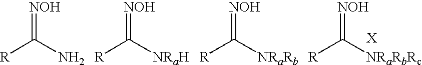
or tautomers thereof, wherein R, Rₐ, R_{b} and R_{c} are independently alkyl, heteroalkyl, aryl or heteroaryl.

12. The method of claim 9, wherein the one or more compounds having at least one amidoxime functional group have the following structure: 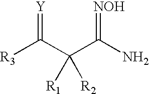
wherein R₁, R₂ and R₃ are independently hydrogen, heteroatoms, heterogroups, alkyl, heteroalkyl, aryl or heteroaryl; and wherein Y is O, NH or NOH.

13. The method of claim 9, wherein the one or more compounds having at least one amidoxime functional group have the following structure: 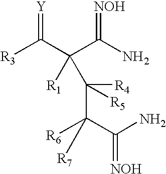
wherein R₁, R₂ and R₃ are independently hydrogen, heteroatoms, heterogroups, alkyl, heteroalkyl, aryl or heteroaryl,
wherein Y is O, NH or NOH, and
wherein R₄, R₅, R₆ and R₇ are independently hydrogen, heteroatoms, heterogroups, alkyl, heteroalkyl, aryl or heteroaryl.

14. The method of claim 11, wherein the one or more compounds having at least one amidoxime functional group are selected from the group consisting of 1,2,3,4,5,6-hexakis-O-[3-(hydroxyamino)-3-iminopropyl Hexitol; 3,3′,3″,3′″-(ethane-1,2-diylbis(azanetriyl))tetrakis(N′-hydroxypropanimidamide); 3,3′-(ethane-1,2-diylbis(oxy))bis(N′-hydroxypropanimidamide); 3-(diethylamino)-N′-hydroxypropanimidamide; 3,3′-(piperazine-1,4-diyl)bis(N′-hydroxypropanimidamide); 3-(2-ethoxyethoxy)-N′-hydroxypropanimidamide; 3-(2-(2-(dimethylamino)ethoxy)ethoxy)-N′-hydroxypropanimidamide; N′-hydroxy-3-(phenylamino)propanimidamide: 3,3′,3″-nitrilotris(N′-hydroxypropanimidamide); 3,3′-(2,2-bis((3-(hydroxyamino)-3-iminopropoxy)methyl)propane-1,3-diyl)bis(oxy)bis(N-hydroxypropanimidamide); 3,3′-(2,2′-(methylazanediyl)bis(ethane-2,1-diyl)bis(oxy))bis(N′-hydroxypropanimidamide); N,N-bis(3-amino-3-(hydroxyimino)propyl)acetamide; 3,3′-(2-(N′-hydroxycarbamimidoyl)phenylazanediyl)bis(N′-hydroxypropanimidamide); 3,3-(2,2′-(3-amino-3-(hydroxyimino)propylazanediyl)bis(ethane-2,1-diyl))bis(oxy)bis(N′-hydroxypropanimidamide) and mixtures thereof.

15. The composition of claim 14, wherein the one or more compounds having at least one amidoxime functional group are selected from the group consisting of 3,3′,3″,3′″-(ethane-1,2-diylbis(azanetriyl))tetrakis(N′-hydroxypropanimidamide); 3,3′-(ethane-1,2-diylbis(oxy))bis(N′-hydroxypropanimidamide); 1,2,3,4,5,6-hexakis-O-[3-(hydroxyamino)-3-iminopropyl Hexitol; 3,3′-(2,2-bis((3-(hydroxyamino)-3-iminopropoxy)methyl)propane-1,3-diyl)bis(oxy)bis(N-hydroxypropanimidamide); N′,2-dihydroxyacetimidamide and mixtures thereof.

16. The method of claim 9, wherein the one or more compounds having at least one amidoxime functional group are derived from the reaction of a nitrile with hydroxylamine.

17. The method of claim 1, wherein the one or more compounds containing at least one amidoxime functional group are present in the polishing composition in an amount of about 0.001 percent by weight to about 5 percent by weight.

18. The method of claim 1, wherein the cleaning composition further comprises one or more oxidizers and one or more surface-active agents, and wherein the surface-active agents include at least one member selected from the group consisting of anionic surfactants, Zwitter-ionic surfactants, multi-ionic surfactants, and combinations thereof.

19. The method of claim 9, wherein the surface of the first wafer to be polished is substantially comprised of an oxide, and wherein the cleaning composition, optionally, further comprises H₂O₂ or hydroxylamine.

20. The method of claim 1, wherein the at least one surfactant is selected from the group consisting of sodium salts of polyacrylic acid, potassium oleate, sulfosuccinates, sulfosuccinate derivatives, sulfonated amines, sulfonated amides, sulfates of alcohols, alkylanyl sulfonates, carboxylated alcohols, alkylamino propionic acids, alkyliminodipropionic acids, and combinations thereof; and wherein the surfactant is present in an amount between about 0.001 and about 10 percent by weight of the composition.

21. The method of claim 1, wherein the cleaning composition further comprises a compound with oxidization or reduction potential.

22. The method of claim 1, wherein the composition is further diluted with water prior to applying it to the polishing pad surface.

23. The method of claim 22, wherein the dilution factor is from about 10 to 500.
